(19) United States
(12) Patent Application Publication
(10) Pub. No.: US 2024/0390369 A1
(43) Pub. Date: Nov. 28, 2024

(54) METHODS FOR DETERMINING RESPONSIVENESS TO TYK2 INHIBITORS

(71) Applicant: Bristol-Myers Squibb Company, Princeton, NJ (US)

(72) Inventors: Yanhua Hu, Princeton, NJ (US); Lu Gao, Princeton, NJ (US); Ian MacQuarie Catlett, Pennington, NJ (US); Xiang Guo, Potomac, MD (US)

(21) Appl. No.: 18/696,915

(22) PCT Filed: Sep. 29, 2022
(86) PCT No.: PCT/US2022/045187

§ 371 Date: Mar. 28, 2024

Related U.S. Application Data
(60) Provisional application No. 63/330,308, filed on Apr. 12, 2022.
(60) Provisional application No. 63/257,407, filed on Oct. 19, 2021.
(60) Provisional application No. 63/250,735, filed on Sep. 30, 2021.

(51) Int. Cl. A61K 31/501; A61P 19/02; G01N 33/68

(52) U.S. Cl. CPC A61K 31/501; A61P 19/02; G01N 33/6869; G01N 33/6893; G01N 2800/52

(57) ABSTRACT

Disclosed are methods of treating psoriatic arthritis in a subject comprising administering to the subject a TYK2 inhibitor (e.g., deucravacitinib), wherein the methods depend on whether the subject exhibits certain levels of specific proteins in the blood (e.g., plasma or serum) prior to or early during administration of the TYK2 inhibitor. Also disclosed are methods for selecting subjects suffering from psoriatic arthritis for treatment with a TYK2 inhibitor, wherein subjects are selected based on the level of one or more proteins in the blood prior to treatment with a TYK2 inhibitor.

### CROSS-REFERENCE TO RELATED APPLICATIONS

This application claims priority to U.S. Provisional Application No. 63/250,735, filed Sep. 30, 2021, U.S. Provisional Application No. 63/257,407, filed Oct. 19, 2021, and U.S. Provisional Application No. 63/330,308, filed Apr. 12, 2022, each of which is hereby incorporated by reference in its entirety herein.

### FIELD OF THE INVENTION

The present invention generally relates to methods of treating psoriatic arthritis in a subject, wherein the methods comprise administering a tyrosine kinase 2 (TYK2) inhibitor to a subject and depend on whether the subject shows certain levels of specific proteins in the blood prior to or early during administration of the TYK2 inhibitor. The invention also relates to methods of selecting a subject having psoriatic arthritis for treatment with a TYK2 inhibitor based on the level of one or more specific proteins in the subject's blood prior to treatment with the TYK2 inhibitor. Embodiments of the invention further relate to administering deucravacitinib to the subject.

### BACKGROUND OF THE INVENTION

Inflammatory and autoimmune diseases such as arthritis, inflammatory bowel disease, psoriasis, and psoriatic arthritis are prevalent and problematic ailments. Such conditions are often chronic or recurring and require long-term treatment to ameliorate symptoms. However, many of the therapies available to treat these diseases do not provide an adequate clinical response in all patients who receive them. In addition, for patients who do not adequately respond to therapy, significant expenditures in their treatment may be incurred before a determination of responsiveness can be made. The ability to select an effective therapy early after diagnosis would greatly benefit patients suffering from these conditions, as well as benefit society, in terms of health-care and other costs and burdens.

For patients with psoriatic arthritis, delay in effective treatment can significantly impact quality of life and functional ability, given the progressive and destructive nature of the disease and its comorbidities (such as cardiovascular disease, osteoporosis, and metabolic syndrome). Psoriatic arthritis can occur after the development of psoriasis, and patients with psoriatic arthritis often present a variety of phenotypes and disease manifestations. The Classification Criteria for Psoriatic Arthritis (CASPAR) include features typical of psoriatic arthritis such as psoriasis, nail disease, dactylitis, and negative serology for rheumatoid factor.

There exists a need in the art to develop new therapeutic strategies for the treatment of psoriatic arthritis that can be used, for example, to predict the response of a patient to a therapy prior to or early during administration of the therapy.

The present invention addresses such need. The present invention provides treatment strategies that can be used to identify patients whose psoriatic arthritis is susceptible to a therapy, namely a therapy comprising a TYK2 inhibitor. A goal of the invention is to provide a more effective and/or better tolerated treatment for patients suffering from psoriatic arthritis, by identifying patients whose psoriatic arthritis is responsive to a TYK2 inhibitor. Such treatment strategies can, for example, increase the probability of patients benefiting from TYK2 inhibitor therapy.

### SUMMARY OF THE INVENTION

Certain aspects of the invention provide a method for identifying a disease in a subject that is susceptible to treatment with a TYK2 inhibitor, comprising determining the level of one or more specific proteins in the subject's blood (e.g., whole blood, serum, or plasma), whereby a protein level above a specified threshold for that protein indicates the disease is susceptible to treatment with a TYK2 inhibitor. Such method may further comprise administering a TYK2 inhibitor to subjects whose disease is identified as being susceptible to treatment with a TYK2 inhibitor. Such method can improve the effectiveness of TYK2 inhibitor treatment, by allowing for the administration of a TYK2 inhibitor to a subset of subjects whose disease will respond better to the TYK2 inhibitor compared to the response in a population of subjects with the disease (such population including subjects who are not part of the subset). In embodiments described herein, the disease is psoriatic arthritis.

Accordingly, certain embodiments of the invention relate to methods for selecting a psoriatic arthritis patient for treatment with a TYK2 inhibitor, the method comprising: (a) measuring (or obtaining a measurement of) the level of one or more proteins in a blood sample from the patient, wherein the one or more proteins are selected from β-defensin 2, interleukin (IL)-19, and IL-17A; (b) for each level of protein measured in (a), comparing the level to a threshold level for the protein; and (c) selecting the patient for treatment with a TYK2 inhibitor if the level of protein measured in (a) for at least one of the one or more proteins is greater than the threshold level for that protein. As demonstrated herein, a protein level above the threshold indicates that the patient's psoriatic arthritis is susceptible to treatment with a TYK2 inhibitor. The one or more proteins measured in (a) may comprise any single protein, or any combination of proteins, selected from β-defensin 2, IL-19, and IL-17A. Such protein(s) may be measured in a serum sample or in a plasma sample from the patient.

In some embodiments, the method further comprises measuring the level of C-reactive protein in the blood sample, and comparing the level of C-reactive protein in the blood sample to a threshold level for C-reactive protein, and selecting the patient for treatment with a TYK2 inhibitor if the level of β-defensin 2, IL-19, and/or IL-17A is above the respective protein's threshold level, and if the level of C-reactive protein is above the threshold level for C-reactive protein. In certain embodiments of the methods described herein, a patient selected for treatment with a TYK2 inhibitor has a level of C-reactive protein in the blood that is above a predetermined threshold level for C-reactive protein.

In any of the aforementioned embodiments, the method may further comprise administering a TYK2 inhibitor to the patient if the patient is selected for treatment with a TYK2 inhibitor. In addition, in any of the aforementioned embodiments, the TYK2 inhibitor may be deucravacitinib.

Embodiments of the invention also relate to methods for treating psoriatic arthritis in a subject (e.g., a patient diagnosed with psoriatic arthritis), comprising: (a) measuring the level of one or more proteins selected from β-defensin 2, IL-19, and IL-17A in a blood sample from the subject (e.g., a serum sample or plasma sample from the subject), (b) for each level of protein measured in (a), comparing that level to a threshold level for the protein, and (c) if the level of at least one protein measured in (a) is above the threshold level for that protein, administering the TYK2 inhibitor to the subject. In further embodiments, for example, at least two proteins are measured in (a), and the TYK2 inhibitor is administered to the subject if, for each of the two proteins, the level measured in the blood sample is above the threshold level for the protein. In certain embodiments, the TYK2 inhibitor is deucravacitinib.

Accordingly, in some embodiments as described herein, the method comprises measuring (or obtaining a measurement of) the level of two of the proteins (e.g., β-defensin 2 and IL-19), and the subject is selected for treatment with a TYK2 inhibitor, or is administered a TYK2 inhibitor, if, for each of the two proteins, the level measured in the sample is above the threshold level for the protein (e.g., the level of β-defensin 2 measured in the sample is above the threshold level for β-defensin 2, and the level of IL-19 measured in the sample is above the threshold level for IL-19). In certain embodiments, the method may comprise measuring the level of all three proteins (i.e., β-defensin 2, IL-19, and IL-17A), and the subject is selected for treatment with a TYK2 inhibitor, or is administered a TYK2 inhibitor, if, for any two of the three proteins, the level measured is above the protein's threshold level.

In some embodiments, the invention provides methods for selecting a psoriatic arthritis patient for TYK2 inhibitor therapy, the method comprising comparing the patient's blood level of β-defensin 2, IL-19, and/or IL-17A to a threshold level for the protein (each protein having its respective threshold level), and selecting the patient for TYK2 inhibitor therapy if the blood level of β-defensin 2, IL-19, and/or IL-17A is above the threshold level. Further embodiments of such methods also comprise administering a TYK2 inhibitor to such patient selected for TYK2 inhibitor therapy.

In certain embodiments, the threshold level for a given protein may be a predetermined level based on, for example, the levels of that protein measured in (i) a population of subjects with psoriatic arthritis and previously treated with the TYK2 inhibitor and shown to be responsive to the TYK2 inhibitor, (ii) a population of subjects with psoriatic arthritis and previously treated with the TYK2 inhibitor and shown to be unresponsive to the TYK2 inhibitor, and/or (iii) a population of subjects with psoriatic arthritis and who have not received TYK2 inhibitor therapy. For example, in some embodiments, the threshold level for β-defensin 2 may be based on the median of the β-defensin 2 levels measured in blood samples from a population of patients having psoriatic arthritis and who have not received TYK2 inhibitor therapy.

### BRIEF DESCRIPTION OF THE DRAWINGS

FIG. 1A provides boxplots of serum β-defensin 2 (BD2) levels (log 2 value) in normal healthy volunteers (NHV) and in PsA patients at baseline, as described in the Examples.

FIG. 1B is a scatter plot showing baseline BD2 level (log 2 value, ng/L) on the x-axis and baseline PASI score on the y-axis, as measured in PsA patients as described in the Examples.

FIG. 2A provides boxplots of serum IL-19 levels (log 2 value) in normal healthy volunteers (NHV) and in PsA patients at baseline, as described in the Examples.

FIG. 2B is a scatter plot showing baseline IL-19 level (log 2 value, ng/L) on the x-axis and baseline PASI score on the y-axis, as measured in PsA patients as described in the Examples.

FIG. 3A provides boxplots of serum IL-17A levels (log 2 value) in normal healthy volunteers (NHV) and in psoriatic arthritis (PsA) patients at baseline, as described in the Examples.

FIG. 3B is a scatter plot showing baseline IL-17A level (log 2 value, ng/L) on the x-axis and baseline PASI score on the y-axis, as measured in PsA patients as described in the Examples.

FIG. 4 provides boxplots of baseline PASI scores for PASI75 non-responders and PASI75 responders, in each treatment arm (placebo, 6 mg deucravacitinib QD, and 12 mg deucravacitinib QD), as described in the Examples. QD=once daily administration of the specified dose (6 mg deucravacitinib or 12 mg deucravacitinib).

FIG. 5 provides boxplots of baseline serum BD2 levels (ng/L) for PASI75 non-responders and PASI75 responders, in each treatment arm (placebo, 6 mg deucravacitinib QD, and 12 mg deucravacitinib QD), as described in the Examples.

FIG. 6 provides boxplots of baseline serum BD2 levels (ng/L) for ACR20 non-responders and ACR20 responders, in each treatment arm (placebo, 6 mg deucravacitinib QD, and 12 mg deucravacitinib QD), as described in the Examples.

FIG. 7 is a bargraph showing PASI75 response rate at week sixteen in each treatment arm, for patients categorized according to baseline BD2 level, as described in the Examples. The group “All patients” includes BD2-high and BD2-low patients, while the group “BD2-high” includes patients whose baseline BD2 level was above the median 9,265 ng/L, and the “BD2-low” group includes patients whose baseline BD2 level was below the median 9,265 ng/L. The y-axis provides PASI75 response rate, and the percent response rate for each treatment arm, within each group, is shown at the top of the bars (with each error bar representing the 95% confidence interval).

FIG. 8 is a bargraph showing ACR20 response rate at week sixteen in each treatment arm, for patients categorized according to baseline BD2 level, as described in the Examples. The group “All patients” includes BD2-high and BD2-low patients, while the group “BD2-high” includes patients whose baseline BD2 level was above the median 9,265 ng/L, and the group “BD2-low” includes patients whose baseline BD2 level was below the median 9,265 ng/L. The y-axis provides ACR20 response rate, and the percent response rate for each treatment arm, within each group, is shown at the top of the bars (with each error bar representing the 95% confidence interval).

FIG. 9 provides line graphs of the PASI75 response rate in each treatment arm (placebo, 6 mg deucravacitinib QD, and 12 mg deucravacitinib QD) over the sixteen-week treatment period, in BD2-high (left line graph) and BD2-low (right line graph) patient groups, as described in the Examples. Error bars represent the 95% confidence intervals.

FIG. 10 provides line graphs of the ACR20 response rate in each treatment arm (placebo, 6 mg deucravacitinib QD, and 12 mg deucravacitinib QD) over the sixteen-week treatment period, in BD2-high (left line graph) and BD2-low (right line graph) patient groups, as described in the Examples. Error bars represent the 95% confidence intervals.

FIG. 11 is a bargraph showing PASI75 response rate at week sixteen in each treatment arm when patients are categorized according to baseline BD2 level (above or below median) and baseline PASI score (above or below median) into four groups, as described in the Examples: BD2-high and PASI-high; BD2-high and PASI-low; BD2-low and PASI-high; and BD2-low and PASI-low. The group “All patients” includes all of the patients from the four groups. The PASI75 response rate is provided at the top of each bar, with error bars representing the 95% confidence intervals.

FIG. 12 provides boxplots of baseline serum IL-19 levels (ng/L) for PASI75 non-responders and PASI75 responders, in each treatment arm (placebo, 6 mg deucravacitinib QD, and 12 mg deucravacitinib QD), as described in the Examples.

FIG. 13 provides boxplots of baseline serum IL-19 levels (ng/L) for ACR20 non-responders and ACR20 responders, in each treatment arm (placebo, 6 mg deucravacitinib QD, and 12 mg deucravacitinib QD), as described in the Examples.

FIG. 14 is a bargraph showing PASI75 response rate at week sixteen in each treatment arm, for patients categorized according to baseline IL-19 level, as described in the Examples. The group “All patients” includes IL-19-high and IL-19-low patients, while the group “IL-19-high” includes patients whose baseline IL-19 level was above the median 36 ng/L, and the group “IL-19-low” includes patients whose baseline IL-19 level was below the median 36 ng/L. The y-axis provides PASI75 response rate, and the percent response rate for each treatment arm, within each group, is shown at the top of the bars (with each error bar representing the 95% confidence interval).

FIG. 15 is a bargraph showing ACR20 response rate at week sixteen in each treatment arm, for patients categorized according to baseline IL-19 level, as described in the Examples. The group “All patients” includes IL-19-high and IL-19-low patients, while the group “IL-19 high” includes patients whose baseline IL-19 level was above the median 36 ng/L, and the group “IL-19 low” includes patients whose baseline IL-19 level was below the median 36 ng/L. The y-axis provides ACR20 response rate, and the percent response rate for each treatment arm, within each group, is shown at the top of the bars (with each error bar representing the 95% confidence interval).

FIG. 16 provides line graphs of the PASI75 response rate in each treatment arm (placebo, 6 mg deucravacitinib QD, and 12 mg deucravacitinib QD) over the sixteen-week treatment period, in IL-19-high (left line graph) and IL-19-low (right line graph) patient groups, as described in the Examples. Error bars represent the 95% confidence interval.

FIG. 17 provides line graphs of the ACR20 response rate in each treatment arm (placebo, 6 mg deucravacitinib QD, and 12 mg deucravacitinib QD) over the sixteen-week treatment period, in IL-19-high (left line graph) and IL-19-low (right line graph) patient groups, as described in the Examples. Error bars represent the 95% confidence intervals.

FIG. 18 is a bargraph showing PASI75 response rate at week sixteen in each treatment arm when patients are categorized according to baseline IL-19 level (above or below median) and baseline PASI score (above or below median) into four groups, as described in the Examples: IL-19-high and PASI-high; IL-19-high and PASI-low; IL-19-low and PASI-high; and IL-19-low and PASI-low. The group “All patients” includes all of the patients from the four groups. The PASI75 response rate is provided at the top of each bar, with error bars representing the 95% confidence intervals.

FIG. 19 provides boxplots of baseline serum IL-17A levels (ng/L) for PASI75 non-responders and PASI75 responders, in each treatment arm (placebo, 6 mg deucravacitinib QD, and 12 mg deucravacitinib QD), as described in the Examples.

FIG. 20 provides boxplots of baseline serum IL-17A levels (ng/L) for ACR20 non-responders and ACR20 responders, in each treatment arm (placebo, 6 mg deucravacitinib QD, and 12 mg deucravacitinib QD), as described in the Examples.

FIG. 21 is a bargraph showing PASI75 response rate at week sixteen in each treatment arm, for patients categorized according to baseline IL-17A level, as described in the Examples. The group “All patients” includes IL-17A-high and IL-17A-low patients, while the group “IL-17A high” includes patients whose baseline IL-17A level was above the median 0.575 ng/L, and the group “IL-17A low” includes patients whose baseline IL-17A level was below the median 0.575 ng/L. The y-axis provides PASI75 response rate, and the percent response rate for each treatment arm, within each group, is shown at the top of the bars (with each error bar representing the 95% confidence interval).

FIG. 22 is a bargraph showing ACR20 response rate at week sixteen in each treatment arm, for patients categorized according to baseline IL-17A level, as described in the Examples. The group “All patients” includes IL-17A-high and IL-17A-low patients, while the group “IL-17A high” includes patients whose baseline IL-17A level was above the median 0.575 ng/L, and the group “IL-17A low” includes patients whose baseline IL-17A level was below the median 0.575 ng/L. The y-axis provides ACR20 response rate, and the percent response rate for each treatment arm, within each group, is shown at the top of the bars (with each error bar representing the 95% confidence interval).

FIG. 23 provides line graphs of the PASI75 response rate in each treatment arm (placebo, 6 mg deucravacitinib QD, and 12 mg deucravacitinib QD) over the sixteen-week treatment period, in IL-17A-high (left line graph) and IL-17A-low (right line graph) patient groups, as described in the Examples. Error bars represent the 95% confidence intervals.

FIG. 24 provides line graphs of the ACR20 response rate in each treatment arm (placebo, 6 mg deucravacitinib QD, and 12 mg deucravacitinib QD) over the sixteen-week treatment period, in IL-17A-high (left line graph) and IL-17A-low (right line graph) patient groups, as described in the Examples. Error bars represent the 95% confidence intervals.

FIG. 25 is a bargraph showing PASI75 response rate at week sixteen in each treatment arm when patients are categorized according to baseline IL-17A level (above or below median) and baseline PASI score (above or below median) into four groups, as described in the Examples: IL-17A-high and PASI-high; IL-17A-high and PASI-low; IL-17A-low and PASI-high; and IL-17A-low and PASI-low. The group “All patients” includes all of the patients from the four groups. The PASI75 response rate is provided at the top of each bar, with error bars representing the 95% confidence intervals.

FIG. 26 is a forest plot providing odds ratio results for ACR20 response, comparing 6 mg QD versus placebo, for each of the subgroups listed in the left-hand column and as described in the Examples. “N” for “Placebo” represents the number of ACR20 responders from the placebo arm, and “N” for “Deucrava” represents the number of ACR20 responders from the 6 mg QD deucravacitinib treatment arm. For example, there were twenty-two PsA patients in the 6 mg QD deucravacitinib treatment arm that achieved ACR20 response and that were categorized in the “BD2 high” group.

FIG. 27 is a forest plot providing odds ratio results for ACR20 response, comparing 12 mg QD versus placebo, for each of the subgroups listed in the left-hand column and as described in the Examples. “N” for “Placebo” represents the number of ACR20 responders from the placebo arm, and “N” for “Deucrava” represents the number of ACR20 responders from the 12 mg QD deucravacitinib treatment arm.

FIG. 28 is a forest plot providing odds ratio results for ACR20 response, comparing deucravacitinib treatment (both treatment arms combined) versus placebo, for each of the subgroups listed in the left-hand column and as described in the Examples. “N” for “Placebo” represents the number of ACR20 responders from the placebo arm, and “N” for “Deucrava” represents the number of ACR20 responders from the combined deucravacitinib treatment arms.

FIG. 29 is a forest plot providing odds ratio results for PASI75 response, comparing 6 mg QD versus placebo, for each of the subgroups listed in the left-hand column and as described in the Examples. “N” for “Placebo” represents the number of PASI75 responders from the placebo arm, and “N” for “Deucrava” represents the number of PASI75 responders from the 6 mg QD deucravacitinib treatment arm.

FIG. 30 is a forest plot providing odds ratio results for PASI75 response, comparing 12 mg QD versus placebo, for each of the subgroups listed in the left-hand column and as described in the Examples. “N” for “Placebo” represents the number of PASI75 responders from the placebo arm, and “N” for “Deucrava” represents the number of PASI75 responders from the 12 mg QD deucravacitinib treatment arm.

FIG. 31 is a forest plot providing odds ratio results for PASI75 response, comparing deucravacitinib treatment (both arms combined) versus placebo, for each of the subgroups listed in the left-hand column and as described in the Examples. “N” for “Placebo” represents the number of PASI75 responders from the placebo arm, and “N” for “Deucrava” represents the number of PASI75 responders from the combined deucravacitinib treatment arms.

FIG. 32 is a forest plot providing odds ratio results for PASI75 response, adjusted for baseline PASI score, comparing 6 mg QD versus placebo for each of the subgroups listed in the left-hand column and as described in the Examples. “N” for “Placebo” represents the number of PASI75 responders from the placebo arm, and “N” for “Deucrava” represents the number of PASI75 responders from the 6 mg QD deucravacitinib treatment arm.

FIG. 33 is a forest plot providing odds ratio results for PASI75 response, adjusted for baseline PASI score, comparing 12 mg QD versus placebo for each of the subgroups listed in the left-hand column and as described in the Examples. “N” for “Placebo” represents the number of PASI75 responders from the placebo arm, and “N” for “Deucrava” represents the number of PASI75 responders from the 12 mg QD deucravacitinib treatment arm.

FIG. 34 is a forest plot providing odds ratio results for ACR20 response, adjusted for baseline DAS28, comparing 6 mg QD versus placebo for each of the subgroups listed in the left-hand column and as described in the Examples. “N” for “Placebo” represents the number of ACR20 responders from the placebo arm, and “N” for “Deucrava” represents the number of ACR20 responders from the 6 mg QD deucravacitinib treatment arm.

FIG. 35 is a forest plot providing odds ratio results for ACR20 response, adjusted for baseline DAS28, comparing 12 mg QD versus placebo for each of the subgroups listed in the left-hand column and as described in the Examples. “N” for “Placebo” represents the number of ACR20 responders from the placebo arm, and “N” for “Deucrava” represents the number of ACR20 responders from the 12 mg QD deucravacitinib treatment arm.

FIG. 36 provides boxplots of baseline serum CRP levels (mg/L) for PASI75 non-responders and PASI75 responders, in each treatment arm (placebo, 6 mg deucravacitinib QD, and 12 mg deucravacitinib QD), as described in the Examples.

FIG. 37 provides boxplots of baseline serum CRP levels (mg/L) for ACR20 non-responders and ACR20 responders, in each treatment arm (placebo, 6 mg deucravacitinib QD, and 12 mg deucravacitinib QD), as described in the Examples

FIG. 38 is a bargraph showing PASI75 response rate at week sixteen in each treatment arm, for patients categorized according to baseline CRP level, as described in the Examples. The group “All patients” includes CRP-high and CRP-low patients, while the group “CRP high” includes patients whose baseline CRP level was above the median 8.31 mg/L, and the group “CRP low” includes patients whose baseline CRP level was below the median 8.31 mg/L. The y-axis provides PASI75 response rate, and the percent response rate for each treatment arm, within each group, is shown at the top of the bars (with each error bar representing the 95% confidence interval).

FIG. 39 is a bargraph showing ACR20 response rate at week sixteen in each treatment arm, for patients categorized according to baseline CRP level, as described in the Examples. The group “All patients” includes CRP-high and CRP-low patients, while the group “CRP high” includes patients whose baseline CRP level was above the median 8.31 mg/L, and the group “CRP low” includes patients whose baseline CRP level was below the median 8.31 mg/L. The y-axis provides ACR20 response rate, and the percent response rate for each treatment arm, within each group, is shown at the top of the bars (with each error bar representing the 95% confidence interval).

FIG. 40 provides line graphs of the PASI75 response rate in each treatment arm (placebo, 6 mg deucravacitinib QD, and 12 mg deucravacitinib QD) over the sixteen-week treatment period, in CRP-high (left line graph) and CRP-low (right line graph) patient groups, as described in the Examples. Error bars represent the 95% confidence intervals.

FIG. 41 provides line graphs of the ACR20 response rate in each treatment arm (placebo, 6 mg deucravacitinib QD, and 12 mg deucravacitinib QD) over the sixteen-week treatment period, in CRP-high (left line graph) and CRP-low (right line graph) patient groups, as described in the Examples. Error bars represent the 95% confidence intervals.

FIG. 42 is a bargraph showing PASI75 response rate at week sixteen in each treatment arm when patients are categorized according to baseline CRP level (above or below median) and baseline PASI score (above or below median) into four groups, as described in the Examples: CRP-high and PASI-high; CRP-high and PASI-low; CRP-low and PASI-high; and CRP-low and PASI-low. The group “All patients” includes all of the patients from the four groups. The PASI75 response rate is provided at the top of each bar, with error bars representing the 95% confidence interval.

FIG. 43A provides line graphs showing percent change from baseline in serum BD2 level for each treatment arm, over the sixteen-week treatment period, as described in the Examples. The y-axis provides the least-square mean, with error bars representing the standard error of percent change from baseline. ***P<0.001 versus baseline; LS, least squares; SE, standard error.

FIG. 43B provides line graphs showing change from baseline in serum BD2 level for each treatment arm, over the sixteen-week treatment period, for PASI75 responders and non-responders. The y-axis provides the adjusted least square mean and standard error of the change from baseline. In the 12 mg QD treatment arm, greater reductions in BD2 serum level were observed over time in the PASI75 responder group compared with the non-responder group. The asterisks show significant differences between responders and non-responders, *P<0.05, **P<0.01.

FIG. 44 provides line graphs showing percent change from baseline in serum IL-19 level for each treatment arm, over the sixteen-week treatment period, as described in the Examples. The y-axis provides the least-square mean, with error bars representing the standard error of percent change from baseline. ***P<0.001 versus baseline; **P<0.01 versus baseline; *P<0.05 versus baseline; LS, least squares; SE, standard error.

FIG. 45 provides line graphs showing percent change from baseline in serum IL-17A level for each treatment arm, over the sixteen-week treatment period, as described in the Examples. The y-axis provides the least-square mean, with error bars representing the standard error of percent change from baseline. ***P<0.001 versus baseline; *P<0.05 versus baseline; LS, least squares; SE, standard error.

FIG. 46 provides line graphs showing percent change from baseline in serum CRP level, measured over the sixteen-week treatment period for each treatment arm, as described in the Examples. The y-axis provides the least-square mean, with error bars representing the standard error of percent change from baseline. ***P<0.001 versus baseline; *P<0.05 versus baseline; LS, least squares; SE, standard error.

### DETAILED DESCRIPTION OF THE INVENTION

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

The present disclosure relates in part to the identification of proteins, and particular protein levels, that are predictive of the responsiveness of a subject's psoriatic arthritis to therapy comprising a TYK2 inhibitor. Embodiments of the present invention provide methods for determining whether a subject's psoriatic arthritis is susceptible to treatment with a TYK2 inhibitor (such as, e.g., deucravacitinib), the methods comprising determining the level of one or more specific proteins in the subject's blood (e.g., by determining the level of one or more specific proteins in a sample of whole blood, serum, or plasma), whereby a protein level above a specified threshold for that protein indicates the psoriatic arthritis is susceptible to treatment with a TYK2 inhibitor. Such methods may further comprise administering a TYK2 inhibitor to a subject whose psoriatic arthritis is determined to be susceptible to treatment with a TYK2 inhibitor. Accordingly, certain embodiments of the present invention provide methods for treating a subject having psoriatic arthritis, wherein the methods comprise (a) identifying a subject as having psoriatic arthritis that is susceptible to treatment with a TYK2 inhibitor (by a method as described herein), and (b) administering to the subject a TYK2 inhibitor (such as, e.g., deucravacitinib).

TYK2 is a member of the Janus kinase (JAK) family of nonreceptor tyrosine kinases and has been shown to be critical in regulating the signal transduction cascade downstream of receptors for IL-12, IL-23, and type I interferons in both mice (Ishizaki, M. et al., “Involvement of tyrosine kinase-2 in both the IL-12/Th1 and IL-23/Th17 axes in vivo,” J. Immunol., 187:181-189 (2011); Prchal-Murphy, M. et al., “TYK2 kinase activity is required for functional type I interferon responses in vivo,” PLOS One, 7:e39141 (2012)) and humans (Minegishi, Y. et al., “Human tyrosine kinase 2 deficiency reveals its requisite roles in multiple cytokine signals involved in innate and acquired immunity,” Immunity, 25:745-755 (2006)). TYK2-deficient mice are resistant to experimental models of colitis, psoriasis, and multiple sclerosis (Ishizaki, M. et al., “Involvement of tyrosine kinase-2 in both the IL-12/Th1 and IL-23/Th17 axes in vivo,” J. Immunol., 187:181-189 (2011); Oyamada, A. et al., “Tyrosine kinase 2 plays critical roles in the pathogenic CD4 T cell responses for the development of experimental autoimmune encephalomyelitis,” J. Immunol., 183:7539-7546 (2009)). In humans, individuals expressing an inactive variant of TYK2 are protected from multiple sclerosis and possibly other autoimmune disorders (Couturier, N. et al., “Tyrosine kinase 2 variant influences T lymphocyte polarization and multiple sclerosis susceptibility,” Brain, 134:693-703 (2011)). Genome-wide association studies have shown other variants of TYK2 to be associated with autoimmune disorders such as Crohn's disease, psoriasis, systemic lupus erythematosus, and rheumatoid arthritis, further demonstrating the importance of TYK2 in autoimmunity (Ellinghaus, D). et al., “Combined Analysis of Genome-wide Association Studies for Crohn Disease and Psoriasis Identifies Seven Shared Susceptibility Loci,” Am. J. Hum. Genet., 90:636-647 (2012); Graham, D. et al., “Association of polymorphisms across the tyrosine kinase gene, TYK2 in UK SLE families,” Rheumatology (Oxford), 46:927-930 (2007); Eyre, S. et al., “High-density genetic mapping identifies new susceptibility loci for rheumatoid arthritis,” Nat. Genet., 44:1336-1340 (2012)).

The present invention relates to methods for identifying a subject suitable for treatment with a TYK2 inhibitor, such as deucravacitinib. Generally, the subject is suffering from an inflammatory or autoimmune disease such as, for example, psoriatic arthritis (PsA). In some embodiments, the subject's disease is more likely than not to respond to a TYK2 inhibitor such as deucravacitinib.

In certain embodiments, the methods comprise determining the level of one or more proteins in a blood sample (e.g., a serum or plasma sample) from the subject, and comparing that protein level to a threshold level, wherein a protein level above the threshold level indicates the subject's disease is likely to be responsive to a TYK2 inhibitor such as deucravacitinib, and a protein level below the threshold level indicates the subject's disease is not likely to be responsive to a TYK2 inhibitor such as deucravacitinib. The proteins can include one or more of β-defensin 2 (BD2), IL-19, and IL-17A. In certain embodiments, the methods further comprise administering to the subject a TYK2 inhibitor, such as deucravacitinib.

For example, in some embodiments, a subject having psoriatic arthritis that is likely to respond to a TYK2 inhibitor (such as deucravacitinib) has a baseline level of BD2 in the blood that is above a predetermined threshold BD2 level. In certain embodiments, such threshold BD2 level has been predetermined by evaluating the levels BD2 in a population of patients with psoriatic arthritis and who have not been treated with a TYK2 inhibitor. In other embodiments, such threshold BD2 level has been predetermined by evaluating the levels of BD2 in a population of patients with psoriatic arthritis and previously treated with the TYK2 inhibitor and shown to be responsive to the TYK2 inhibitor, and in a population of patients with psoriatic arthritis and previously treated with the TYK2 inhibitor and shown to be unresponsive to the TYK2 inhibitor.

Additional embodiments, similar to the above, are based on a subject's baseline level of IL-19 or IL-17A. For example, in some embodiments, a subject having psoriatic arthritis that is likely to respond to a TYK2 inhibitor (such as deucravacitinib) has a baseline level of IL-19 in the blood that is above a predetermined threshold IL-19 level. Such baseline level of IL-19 may be used in combination with the subject's baseline level of BD2 and/or the subject's baseline level of IL-17A, to determine whether the subject's psoriatic arthritis is likely to respond to a TYK2 inhibitor.

Accordingly, in various embodiments as described herein, the levels of one or more proteins (e.g., one or more of BD2, IL-19, and IL-17A) are assessed to select a subject for treatment with a TYK2 inhibitor (e.g., to identify a subject who is to be administered deucravacitinib or another TYK2 inhibitor). In further embodiments, a composite of two or more proteins (e.g., any two or more of BD2, IL-19, and IL-17A) are used in the methods described herein.

In any of the embodiments described herein, a subject's baseline level of C-reactive protein (CRP) may be used, in combination with the subject's baseline level of BD2, IL-19, and/or IL-17A, to select the subject for treatment with a TYK2 inhibitor, or to otherwise predict or determine whether the subject's disease will be responsive to TYK2 inhibitor therapy.

In any of the embodiments described herein, one or more of a subject's baseline clinical scores may be used, in combination with the subject's baseline level of BD2, IL-19, and/or IL-17A (and optionally CRP level), to select the subject for treatment with a TYK2 inhibitor, or to otherwise predict or determine whether the subject's disease will be responsive to TYK2 inhibitor therapy.

In some embodiments, the present invention provides a method of treating PsA in a subject comprising administering to the subject deucravacitinib, wherein the subject shows a certain specific protein level (or protein levels) in one or more bodily fluid samples (e.g., whole blood, plasma, or serum) prior to administering deucravacitinib, or early during the administration of deucravacitinib. The specific protein may be one or more of the proteins described herein (e.g., BD2, IL-19, and/or IL-17A). As demonstrated herein, high levels of such proteins are associated with improved clinical responses to deucravacitinib administration, and PsA patients with higher levels of one or more of such proteins at baseline (prior to treatment with a TYK2 inhibitor) respond better to deucravacitinib than do PsA patients with comparatively lower protein level(s) at baseline. Accordingly, the level of BD2, IL-19, and/or IL-17A in blood may be used to identify subjects whose psoriatic arthritis will be susceptible to treatment with a TYK2 inhibitor such as deucravacitinib.

In addition, certain embodiments of the present invention provide a method for selecting subjects such as PsA patients for treatment with a TYK2 inhibitor such as deucravacitinib, the method comprising measuring the level of one or more proteins in a patient's blood sample (e.g., a serum or plasma sample), and comparing that protein level to a threshold level, wherein a protein level above the threshold level indicates the patient's PsA is susceptible to treatment with a TYK2 inhibitor such as deucravacitinib, and a protein level below the threshold level indicates the patient's PsA is not susceptible to TYK2 inhibitor treatment. The protein(s) measured can include one or more of β-defensin 2 (BD2), IL-19, and IL-17A. As mentioned above, each of these proteins may be used alone or as part of a composite index to identify PsA patients who are likely to respond to therapy comprising a TYK2 inhibitor such as deucravacitinib. In further embodiments, the level of C-reactive protein (CRP) in the blood may also be taken into account. In certain embodiments, the methods further comprise administering a TYK2 inhibitor, such as deucravacitinib, to those patients with protein level(s) above the threshold(s).

In some embodiments, a PsA patient's baseline Psoriasis Area and Severity Index (PASI) score is used (in combination with levels of one or more proteins described herein) to determine whether the patient's psoriatic arthritis is responsive to, or is likely to respond to, a TYK2 inhibitor (e.g., deucravacitinib). The Psoriasis Area and Severity Index (PASI) is a quantitative rating system for measuring the severity of psoriatic lesions based on area coverage and plaque appearance. The PASI score is used to evaluate baseline and response to therapy in psoriasis and PsA. The response measure PASI75 is a binary outcome that indicates a 75% or greater improvement in PASI score from baseline PASI score.

In certain aspects, the present invention provides a method for identifying a subject having psoriatic arthritis that is susceptible to treatment with a TYK2 inhibitor (e.g., deucravacitinib), comprising determining the level of BD2, IL-19, and/or IL-17A in the subject's blood (e.g., whole blood, serum, or plasma), whereby a high level of BD2, a high level of IL-19, and/or a high level of IL-17A (for each protein, a high level being a level that is above a specified threshold level for that protein) indicates that the subject's psoriatic arthritis is susceptible to treatment with the TYK2 inhibitor. In certain of the aforementioned embodiments, a subject's baseline CRP level and/or baseline PASI score is also used, in combination with the subject's baseline level of BD2, IL-19, and/or IL-17A in the subject's blood, to assess whether the subject's psoriatic arthritis is susceptible to treatment with a TYK2 inhibitor (e.g., deucravacitinib).

In certain embodiments, the threshold level that is used to designate a certain protein level as “high” is determined based on the levels of the specific protein in the blood of a comparison group of subjects, as can be measured in samples (e.g., of whole blood, plasma, or serum) taken from the comparison group of subjects. In some embodiments, the comparison group of subjects is a group of healthy subjects (subjects not diagnosed with psoriatic arthritis or other inflammatory or autoimmune condition). In other embodiments, the comparison group of subjects is a group of subjects diagnosed with psoriatic arthritis. In some such embodiments, the comparison group of subjects is a group of subjects diagnosed with psoriatic arthritis, and the samples (e.g., of whole blood, plasma, or serum) are taken from these subjects prior to any treatment with a TYK2 inhibitor (e.g., prior to any treatment with deucravacitinib).

In some embodiments, the comparison group of subjects includes at least 50, at least 75, at least 100, at least 150, or at least 200 subjects.

In some embodiments, the measured levels of a protein are mathematically transformed (e.g., such as by taking the log 2 value of the measured concentration of protein in the sample).

The threshold level for a protein is typically a predetermined score or value. Such threshold level can be calculated, for example, as a percentile (e.g., 10th percentile, 20th percentile, 30th percentile, 40th percentile, 50th percentile (median), 60th percentile, 70th percentile, 80th percentile, or 90th percentile) or as an average of the levels (e.g., of the mathematically transformed levels) of the specific protein in the blood of the comparison group of subjects, or as otherwise described herein.

In many embodiments, each specific protein has its own threshold level (e.g., a threshold level is determined separately for each specific protein). In other embodiments, the threshold level is a composite score calculated based on values for two or more specific proteins; in such embodiments, a score may be calculated for the patient, based on the patient's blood levels for the two or more specific proteins, and such score for the patient may be compared to the composite score.

Generally, a protein level above the threshold level is designated as “high” while a protein level below the threshold is designated as “low.” In one such embodiment, the threshold level is a median.

In some embodiments, a threshold level for a particular protein is used to categorize subjects into a “low level” group or “high level” group for that protein, wherein subjects whose level is below the threshold level would fall into the “low level” group and subjects whose level is above the threshold level would fall into the “high level” group.

In certain embodiments, the threshold level is determined by listing all of the available levels measured for the protein in a particular population of subjects (e.g., patients with psoriatic arthritis), determining the median from this listing of levels, and taking this median as the threshold level.

In some embodiments, the threshold level is determined from the levels measured for the protein in a population of patients diagnosed with psoriatic arthritis and who have not been administered a TYK2 inhibitor for a minimum time period (e.g., at least 3, 6, 9, 12, or 24 months) prior to sampling.

For example, in embodiments where a median level is used as the threshold level, samples (e.g., blood samples) are taken from subjects (e.g., about 50, 75, 100, 150, 200, or more subjects) meeting certain clinical criteria for psoriatic arthritis and who are not receiving TYK2 inhibitor therapy, and the level of one or more proteins in each sample is determined. Such proteins may include β-defensin 2, IL-19, and IL-17A. The raw data optionally may be subjected to data processing steps. The median of the data (raw data or processed data) for a protein is then used as the threshold level for that protein, and can be used to categorize a subject into a “low level” or “high level” category.

“Low level” or “high level” categories as described herein can be used to determine whether to administer a TYK2 inhibitor to the subject, or whether to select a subject for TYK2 inhibitor therapy.

Accordingly, in some embodiments, a threshold protein level may be used to categorize PsA patients into a “low level” group or “high level” group for a specific protein, where the threshold level is determined by listing all of the available levels for that protein as measured in the blood (e.g., serum) of PsA patients, determining the median from this listing of protein level values, and taking this median value as the threshold level. Such threshold level may be referred to herein as a median threshold. The blood sampling for the evaluation of the median value for the specific protein may be performed prior to treatment of the psoriatic arthritis with a TYK2 inhibitor. PsA patients may be classified as “high level” if their specific protein level in a sample (e.g., of plasma or serum) is higher than the median threshold, while PsA patients may be classified as “low level” if their specific protein level in a sample (e.g., of plasma or serum) is lower than or equal to the median threshold. The specific protein may be one or more of the proteins described herein (e.g., BD2, IL-19, and/or IL-17A).

Methods for measuring protein level in a sample of a bodily fluid (e.g., a whole blood, plasma, or serum sample) are known in the art. These methods include, but are not limited to, immunoassays such as ELISA and variants thereof such as radioimmunoassay (RIA) and Single Molecule Array (Simoa) immunoassay, SDS-polyacrylamide electrophoresis (SDS-PAGE) Mass Spectrometry, Proximity Ligation Assay (PLA) technologies, and the SomaLogic Proteomic Affinity Assay technologies.

As discussed above, in certain embodiments, the threshold level for a particular protein is a predetermined value. Such predetermined value may be based on a median or other value as described above, or may be based on other data analysis or methods. Regardless of the basis of such predetermined value, a subject (e.g., a patient with PsA) may be classified as “high level” if his/her protein level in the blood is higher than the predetermined value, or may be classified as “low level” if his/her protein level in the blood is lower than or equal to the predetermined value.

In some embodiments, the threshold level need not be used to classify subjects into “low level” or “high level” groups but is used to identify a subject (e.g., a patient with PsA) for treatment with a TYK2 inhibitor, and optionally further to administer a TYK2 inhibitor to the subject, as described herein. In further embodiments, the TYK2 inhibitor is deucravacitinib.

Accordingly, as described herein, one or more protein levels in a subject's blood can be used to predict the responsiveness of a subject's PsA to a TYK2 inhibitor such as deucravacitinib. The present invention provides particular protein level-clinical outcome associations that are useful for determining the responsiveness of a subject's PsA to treatment with a TYK2 inhibitor such as deucravacitinib. The present invention in part provides methods and kits for predicting the outcome of treating a subject with a TYK2 inhibitor, based on the level of one or more specific proteins in the subject's blood prior to treatment. Embodiments of the invention further relate to methods for treating a subject having PsA with a TYK2 inhibitor, wherein the subject's PsA has been determined to be susceptible to treatment with a TYK2 inhibitor. In certain embodiments, the method comprises determining the level of one or more proteins in a sample (e.g., a blood sample, such as whole blood, serum, or plasma) from a subject, wherein the one or more of the proteins are selected from BD2, IL-19, and IL-17A, and wherein, for at least one of the one or more proteins whose level is determined, the protein is present in the sample at a level that is above a threshold level for that protein, thereby providing an indication that the subject's PsA is responsive to, or is susceptible to treatment with, a TYK2 inhibitor; further embodiments comprise administering a TYK2 inhibitor to the subject. In certain embodiments, the TYK2 inhibitor is deucravacitinib.

In certain embodiments, being responsive to a TYK2 inhibitor may refer to achieving a particular clinical outcome (e.g., a PASI75 response, an ACR20 response, etc.) after a minimum treatment period (e.g., eight weeks, ten weeks, twelve weeks, fourteen weeks, or sixteen weeks) with a TYK2 inhibitor (such as deucravacitinib). For example, for subjects with PsA, being responsive to a TYK2 inhibitor may refer to achieving a PASI75 response twelve weeks after initiation of treatment with the TYK2 inhibitor; in further embodiments, being responsive to a TYK2 inhibitor may refer to achieving a PASI75 response sixteen weeks after initiation of treatment with the TYK2 inhibitor.

In any of the embodiments described herein, the TYK2 inhibitor can be deucravacitinib. Deucravacitinib is also known as 6-(cyclopropanecarboxamido)-4-((2-methoxy-3-(1-methyl-1H-1,2,4-triazol-3-yl)phenyl) amino)-N-(methyl-d3)pyridazine-3-carboxamide, having the structure of Formula (I):

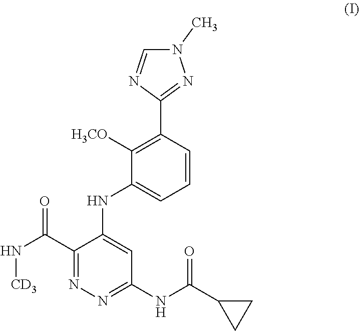 [SMILES: [2H]C([2H])([2H])NC(=O)c1nnc(NC(=O)C2CC2)cc1Nc1cccc(-c2ncn(C)n2)c1OC]

Deucravacitinib is a selective TYK2 inhibitor currently in clinical trials for the treatment of inflammatory and autoimmune diseases such as psoriasis, psoriatic arthritis, lupus, lupus nephritis, Sjögren's syndrome, ulcerative colitis, Crohn's disease, and ankylosing spondylitis. Deucravacitinib is disclosed in U.S. Pat. No. RE47,929 E, which is assigned to the present assignee, and the contents of which are hereby incorporated by reference in their entirety herein. Other TYK2 inhibitors include, for example, TYK2 inhibitors as described in WO 2012/000970, WO 2012/035039, WO 2013/174895, WO 2015/091584, WO 2015/032423, WO 2017/040757, WO 2018/071794, WO 2018/075937, WO 2019/023468, US 2015/0045349, US 2015/0094296, and US 2016/0159773, the contents of each of which are hereby incorporated by reference in their entirety herein.

For any of the embodiments and methods described herein where the TYK2 inhibitor is deucravacitinib, the dose of deucravacitinib that may be administered to a subject can range from about 1 mg to about 40 mg per day. For example, in some embodiments, a dose of 3 mg, 6 mg, 12 mg, 15 mg, or 36 mg deucravacitinib per day is administered to a subject in a method as described herein. Such per day doses may be administered once daily, or may be administered in two or more divided doses (for example, for a total daily dose of 12 mg, the 12 mg may be administered once daily, or may be administered as two 6 mg doses, or may be administered as three 4 mg doses).

In some embodiments, the present invention provides kits for identifying subjects with PsA for treatment with a TYK2 inhibitor. Such kits can be useful for predicting the responsiveness of a subject's PsA to a TYK2 inhibitor. In certain embodiments, the kits comprise components for measuring the level of one or more proteins (e.g., BD2, IL-19, and/or IL-17A) in a sample, and/or components for comparing the level of one or more proteins in a sample to one or more standards, wherein the standards are based on the protein levels measured in a population of patients with the disease and not previously treated with a TYK2 inhibitor. Alternatively, the standards may be based on the protein levels measured in a population of patients with the disease and previously treated with the TYK2 inhibitor and shown to be responsive to the TYK2 inhibitor, and in a population of patients with the disease and previously treated with the TYK2 inhibitor and shown to be unresponsive to the TYK2 inhibitor.

In any of the embodiments described herein, a subject may be administered a TYK2 inhibitor such as deucravacitinib in combination with one or more other agents.

In the context of the present invention, subjects, and in particular human subjects, may also be referred to as patients.

Any definitions set forth herein take precedence over definitions set forth in any patent, patent application, and/or patent application publication incorporated herein by reference. The text of any patent, patent application, patent application publication, or other material that has been incorporated by reference is incorporated by reference only to the extent that no conflict exists between such text and the present specification; in the event of a conflict, any conflicting text is specifically not incorporated by reference.

All measurements are subject to experimental error, consistent with the spirit of the invention.

### Examples

The invention will be further described by the following examples. The examples serve only to illustrate the invention and its practice. The examples are not to be construed as limitations on the scope or spirit of the invention.

In a double-blind Phase 2 trial, 203 patients with psoriatic arthritis were randomized into three groups: deucravacitinib 6 mg once daily (N=70), deucravacitinib 12 mg once daily (N=67), or placebo (N=66). All subjects had active psoriatic arthritis according to the Classification Criteria for Psoriatic Arthritis (CASPAR); for active disease, these criteria required ≥3 tender joint count, ≥3 swollen joint count, and a C-reactive protein level above 3 mg/L. Other inclusion criteria included the following: presence of at least one plaque psoriasis lesion, and prior treatment failure with a non-biologic disease modifying anti-rheumatic drug, a non-steroidal anti-inflammatory drug, or a steroid. With respect to treatment history, approximately 70% of subjects had not received prior treatment with a biologic and were biologic naïve, while up to 30% of subjects had failed or had been intolerant to one TNF-inhibitor (TNF=Tumor necrosis factor, also known as TNFα). Randomization was stratified by prior TNF-inhibitor use (experienced/naïve) and body weight (<90 kg and ≥90 kg).

Levels of proteins in serum were measured at baseline and at various points during sixteen weeks of treatment with deucravacitinib. A number of proteins were examined, and of the proteins examined, β-defensin 2 (BD2), IL-19, and IL-17A were shown to exhibit levels predictive of clinical response to deucravacitinib. Levels of BD2 in serum samples were measured by ELISA; levels of IL-19 and IL-17A in serum were each measured by ultrasensitive Simoa technology.

In addition, comparisons of protein levels between healthy controls (normal healthy volunteers) and PsA patients at baseline (before treatments) were performed. Data from 60 normal healthy volunteers who were matched to PsA patients by age, sex, and weight were used for such comparisons.

Clinical measures included the Psoriasis Area Activity Index (PASI) score for skin psoriasis, American College of Rheumatology 20 (ACR20) response, Psoriatic Arthritis Disease Activity Score (PASDAS), Health Assessment Questionnaire-Disability Index (HAQ-DI), and Disease Activity Score 28 CRP (DAS28). To evaluate the predictive value of different protein measurements for the clinical response to deucravacitinib, baseline protein concentrations for responders and non-responders, as defined by ACR20 or PASI75 at week 16, were compared for each arm (placebo, 6 mg deucravacitinib once-daily, and 12 mg deucravacitinib once-daily).

### Results

### Protein Levels in PsA Patients at Baseline:

Of the proteins evaluated, BD2, IL-19, and IL-17A were found to be predictive of clinical outcomes in PsA patients. In addition, baseline serum concentrations of BD2, IL-19, and IL-17A were elevated in patients with psoriatic arthritis (compared to levels in normal healthy volunteers) and were highly correlated with severity of skin involvement as measured by PASI score.

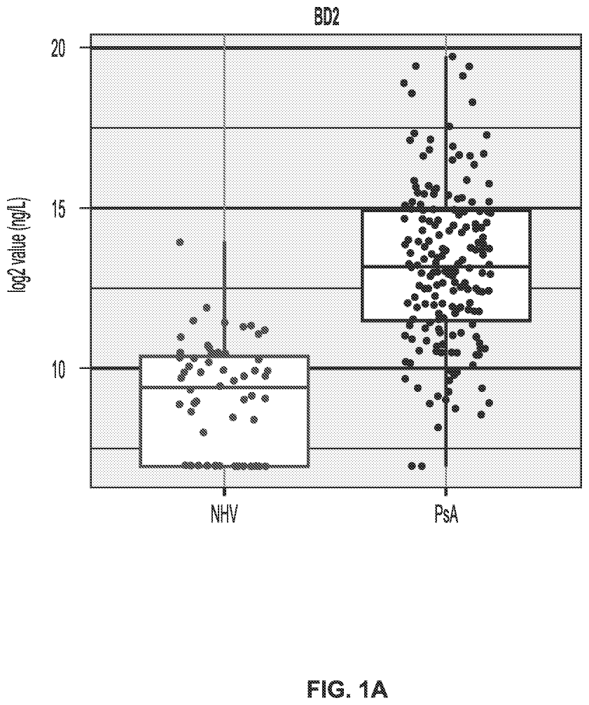

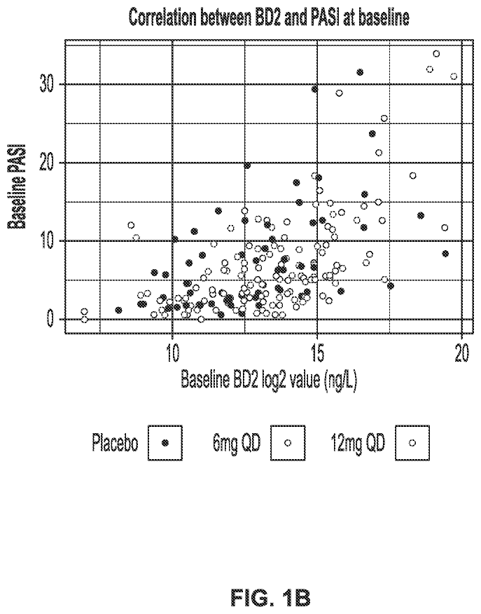

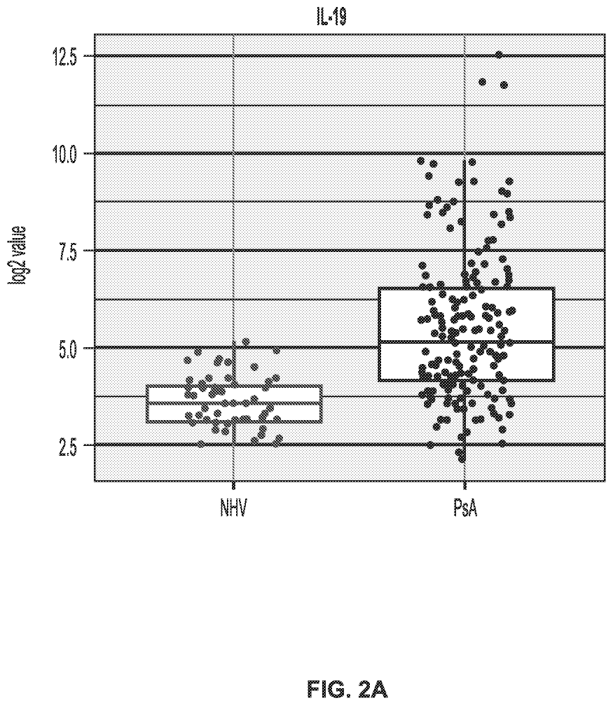

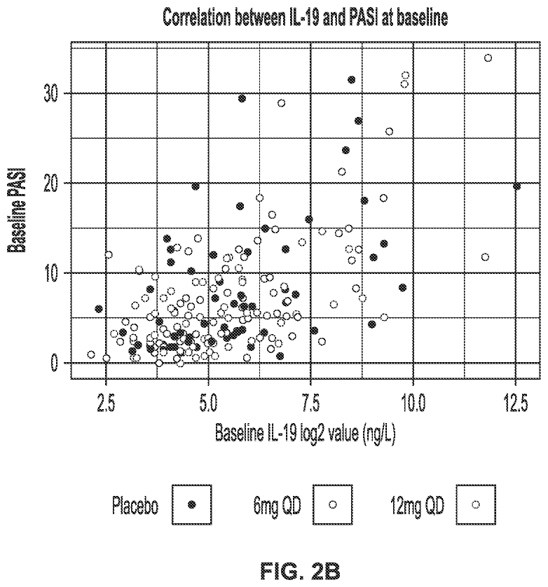

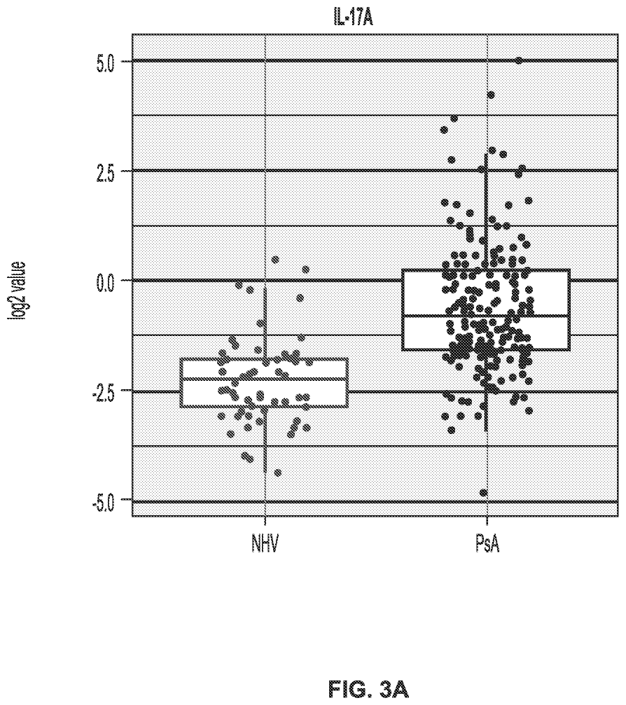

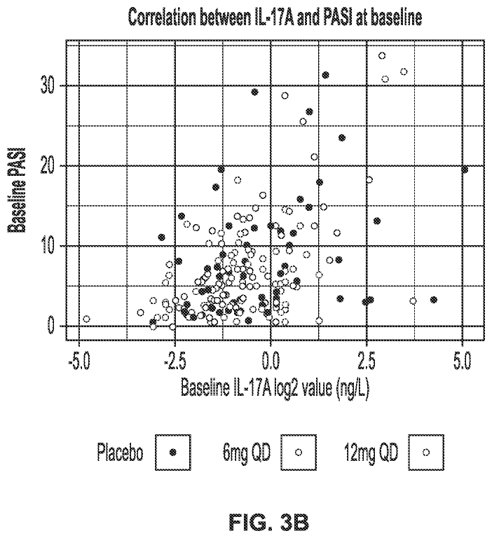

Baseline serum β-defensin 2 levels were significantly higher in PsA patients than in normal healthy volunteers (p<0.0001). FIG. 1A provides box plots for serum β-defensin 2 concentration (log 2 value, y-axis) in normal healthy volunteers (NHV, left box plot) and psoriatic arthritis patients (PsA, right box blot). Baseline serum IL-19 and IL-17A levels were also significantly higher in PsA patients than in normal healthy volunteers (p<0.0001 for each), as shown in FIG. 2A for IL-19 and in FIG. 3A for IL-17A.

For PsA patients at baseline (prior to treatment with deucravacitinib or with placebo), certain protein serum levels correlated with PASI scores. FIG. 1B is a graph showing baseline β-defensin 2 concentration on the x-axis (ng/L, log 2 value) and baseline PASI score on the y-axis; baseline β-defensin 2 levels correlated with baseline PASI scores (Spearman's Rank Correlation Coefficient, or ρ=0.56; p<0.0001). In addition, baseline IL-19 levels correlated with baseline PASI scores (ρ=0.5; p<0.0001), as shown in FIG. 2B, and baseline IL-17A concentrations correlated with baseline PASI scores (ρ=0.4; p<0.0001), as shown in FIG. 3B. Baseline CRP levels did not exhibit as strong of a correlation to baseline PASI score (ρ=0.14; p=0.04).

Pearson's correlation analyses were performed to test for any association between baseline level of BD2, IL-19, or IL-17A, and baseline CRP level in PsA patients. Pearson's correlation analyses also were performed to test for any association between protein serum level and disease activity at baseline, using the following disease activity measurements: PASI, DAS28, HAQ-DI, and PASDAS. Baseline BD2 expression level (log 2 value) was weakly correlated with baseline CRP level (log 2 value), with a correlation coefficient of 0.2 (p-value=0.00526); baseline BD2 expression level (log 2 value) correlated with baseline PASI score, with a correlation coefficient of 0.57 (p-value<0.0001). Baseline BD2 expression did not exhibit a strong or statistically significant correlation with the other disease activity measures at baseline (baseline DAS28, baseline HAQ-DI, or baseline PASDAS, with correlation coefficients of 0.04 (p-value=0.60015), 0.06 (p-value=0.38953), and 0.12 (p-value=0.08699), respectively). Similar results were observed for IL-19 and IL-17A baseline levels. Baseline IL-19 expression (log 2 value) was weakly correlated with baseline CRP level (log 2 value) (correlation coefficient=0.27; p-value=0.000012) and correlated with baseline PASI score (correlation coefficient=0.58; p-value<0.0001), whereas baseline IL-19 expression (log 2 value) did not exhibit a strong correlation with the other activity measures at baseline (with correlation coefficients of 0.12 (p-value=0.08929), 0.15 (p-value=0.03972), and 0.16 (p-value=0.02192) for baseline DAS28, baseline PASDAS, and baseline HAQ-DI, respectively). For IL-17A, baseline IL-17A expression (log 2 value) correlated with baseline CRP level (log 2 value) (correlation coefficient=0.3; p-value=le-05) and with baseline PASI score (correlation coefficient=0.46; p-value<0.0001) but did not exhibit a strong or statistically significant correlation with the other disease activity measures at baseline (with correlation coefficients of 0.06 (p-value=0.40389), 0.14 (p-value=0.04863), and 0.13 (p-value=0.06776) for baseline DAS28, baseline PASDAS, and baseline HAQ-DI, respectively).

### Correlations Between Baseline Clinical Scores and Response Status:

The relationship between baseline PASI score with PASI75 or ACR20 response status at week 16, and the relationship between baseline DAS28 with ACR20 response at week 16, were evaluated. The medians for baseline PASI score and baseline DAS28 were 6.6 and 5.1, respectively. The median of baseline PASI score was calculated from the 165 subjects who had PASI75 response data (see Table 1 below). For each treatment arm, a two-sample t-test was performed to test whether the mean baseline score was different in responders versus non-responders. A Wilcoxon rank sum test (Mann-Whitney U test) was also performed for each treatment arm, to test the difference in baseline median score between responders and non-responders.

TABLE 1
Number of subjects with protein level and response data

| | Total number of | PASI75 response | ACR20 response |
|---|---|---|---|
| Protein | subjects | data | data |
| BD2 | 200 | 162 | 146 |
| IL-19 | 201 | 163 | 147 |
| IL-17A | 202 | 164 | 148 |
| CRP | 203 | 165 | 149 |

In addition, a logistic regression model was fit, to determine whether baseline disease score (dichotomized by the median baseline score, into baseline score high and baseline score low groups) was associated with response status. The model generated an odds ratio for each dosed arm versus placebo, in baseline score high and baseline score low groups. A p-value of the interaction term was used to identify whether the odds ratio (OR) was significantly different between baseline score high and baseline score low groups.
For assessing the relationship between baseline PASI score and PASI75 response, the model was: PASI75 (Y/N)=treatment arm+PASI baseline group+treatment arm*PASI baseline group+TNF-inhibitor use+baseline weight (as used herein, “baseline weight” refers to the weight of the subject in kg (continuous)).
For assessing the relationship between baseline PASI score and ACR20 response, the model was: ACR20 (Y/N)=treatment arm+PASI baseline group+treatment arm*PASI baseline group+TNF-inhibitor use+baseline weight (continuous).
For assessing the relationship between baseline DAS28 and ACR20 response, the model was: ACR20 (Y/N)=treatment arm+DAS28 baseline group+treatment arm*DAS28 baseline group+TNF-inhibitor use+baseline weight (continuous).

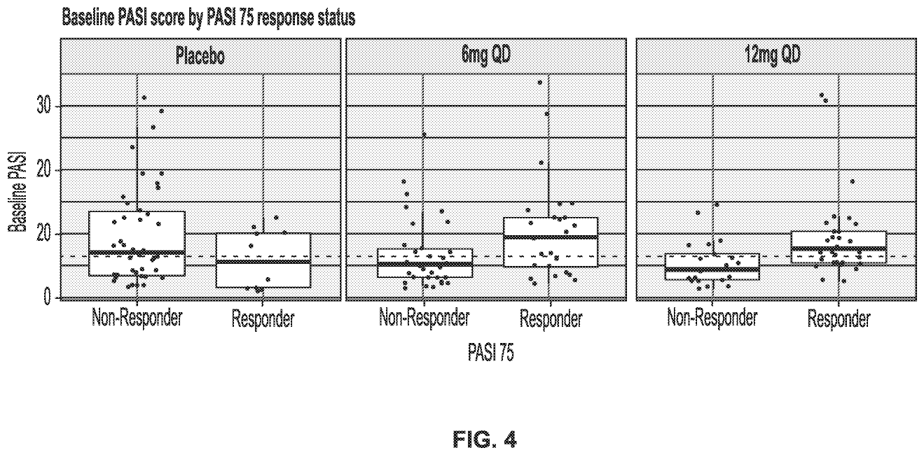

Applying these statistical comparisons and logistic regression models, baseline PASI correlated with PASI75 response status to a statistically significant level, while the other tested relationships did not yield statistically significant associations. FIG. 4 and the tables below provide the data for correlating baseline PASI with PASI75 response status.

FIG. 4 provides boxplots for baseline PASI score by PASI75 response status in each treatment arm, with the solid horizontal line in each box indicating the median value for that box plot, and the dashed horizontal line indicating the baseline PASI median value of 6.6 for all 165 subjects across all of the box plots (the 165 subjects who had PASI75 data). Table 2 provides p-values for the t-test and Wilcoxon rank sum tests described above. Table 3 provides the number of subjects in each treatment arm according to response status (PASI75 responder or non-responder) and baseline score status (baseline PASI score above, or at or below, the baseline PASI score median value of 6.6 for all 165 subjects who had PASI75 data). Table 4 provides odds ratio results for the logistic regression model used to predict PASI75 response from baseline PASI score.

TABLE 2
p-values for each treatment arm, comparing baseline
PASI score in PASI75 responders versus non-responders

| | | Wilcoxon rank sum test |
|---|---|---|
| | t-test comparing baseline | comparing baseline PASI |
| | PASI mean score in | median score in PASI75 |
| Treatment | PASI75 responders versus | responders versus non- |
| arm | non-responders | responders |
| Placebo | 0.04537 | 0.08967 |
| 6 mg QD | 0.04343 | 0.03424 |
| 12 mg QD | 0.00713 | 0.00284 |

TABLE 3
Number of subjects by PASI75 response status and treatment arm

| | | Below/above | |
|---|---|---|---|
| | | baseline PASI | Number of |
| Treatment arm | PASI75 at week 16 | median value of 6.6 | subjects |
| Placebo | Non-responder | Below | 20 |
| | | Above | 23 |
| | Responder | Below | 6 |
| | | Above | 5 |
| 6 mg QD | Non-responder | Below | 23 |
| | | Above | 11 |
| | Responder | Below | 9 |
| | | Above | 16 |
| 12 mg QD | Non-responder | Below | 15 |
| | | Above | 6 |
| | Responder | Below | 11 |
| | | Above | 20 |

TABLE 4
Logistic regression odds ratio results for correlating
PASI75 response status with baseline PASI score

| | | Odds | | | |
|---|---|---|---|---|---|
| | | Ratio or | | | |
| | | Interaction | | Confidence Interval | |
| Group | Comparison | Term | p-value | lower | upper |
| All | 6 mg QD vs | 2.879507 | 0.0139152 | 1.2396003 | 6.688898 |
| | placebo | | | | |
| All | 12 mg QD | 5.781188 | 0.0000692 | 2.4360034 | 13.720069 |
| | vs placebo | | | | |
| Baseline | 6 mg QD vs | 6.937111 | 0.0022656 | 2.0005970 | 24.054574 |
| PASI high | placebo | | | | |
| Baseline | 12 mg QD | 15.751704 | 0.0000533 | 4.1359885 | 59.989574 |
| PASI high | vs placebo | | | | |
| Baseline | 6 mg QD vs | 1.258076 | 0.7080266 | 0.3783530 | 4.183276 |
| PASI low | placebo | | | | |
| Baseline | 12 mg QD | 2.453607 | 0.1429598 | 0.7383291 | 8.153800 |
| PASI low | vs placebo | | | | |
| Interaction | 6 mg QD vs | 5.514064 | 0.0535809 | 0.9740252 | 31.215726 |
| term: | placebo: | | | | |
| Treatment | baseline | | | | |
| arm * | PASI high | | | | |
| baseline | versus PASI | | | | |
| PASI | low | | | | |
| group | | | | | |
| Interaction | 12 mg QD | 6.419815 | 0.0425310 | 1.0646363 | 38.711838 |
| term: | vs placebo: | | | | |
| Treatment | baseline | | | | |
| arm * | PASI high | | | | |
| baseline | versus PASI | | | | |
| PASI | low | | | | |
| group | | | | | |

The odds ratio results presented in Table 4 above provide measures of association between treatment and PASI75 response status, for particular patient groups: all patients, patients having baseline PASI scores above the median 6.6 value (baseline PASI high), and patients having baseline PASI scores at or below the median 6.6 value (baseline PASI low). For the first six rows, each odds ratio (OR) represents the ratio of (i) the odds that PASI75 response will occur given a particular treatment (6 mg QD or 12 mg QD) and (ii) the odds of achieving PASI75 response in the absence of treatment (placebo). An odds ratio greater than 1 indicates that treatment is associated with a higher odds of achieving PASI75 response, whereas an odds ratio less than 1 indicates that treatment is associated with a lower odds of achieving PASI75 response; an odds ratio equal to one indicates that treatment does not affect the odds of achieving PASI75 response. The ORs of the first two rows represent the ORs of comparing 6 mg QD or 12 mg QD versus placebo, for all patients: PASI75 (Y/N)=treatment arm+TNF-inhibitor use+baseline weight (continuous). The ORs of the third through sixth rows provide results from the following: PASI75 (Y/N)=treatment arm+baseline PASI group+treatment arm*baseline PASI group+TNF-inhibitor use+baseline weight (continuous). For example, in the third row, 6.94 is the odds ratio comparing 6 mg QD versus placebo for patients having a baseline PASI score above the median value 6.6. The last two rows of Table 4 provide the interaction terms; for example, in the seventh row, the odds ratio of 5.51 was calculated by taking the ratio of two odds ratios: a) the odds ratio of 6 mg QD versus placebo in the baseline PASI high groups and b) the odds ratio of 6 mg QD versus placebo in the baseline PASI low group (i.e., for comparing the odds ratios 6.94 versus 1.26). These results, presented in Table 4, show that in each treatment arm, the baseline PASI-high group showed a significant treatment benefit (significantly greater odds of achieving PASI75 with deucravacitinib treatment than with placebo).

Correlations between baseline protein level and response status:

Baseline levels of certain serum proteins (BD2, IL-19, and IL-17A) were found to provide predictive value with respect to important clinical outcomes, including ACR20 and PASI75. Boxplots for baseline protein level by response were generated with a horizontal line indicating the median protein level at baseline. For each treatment arm, a two-sample t-test was performed to test whether mean baseline serum protein level was different in responders versus non-responders. A Wilcoxon rank sum test (Mann-Whitney U test) was also performed to evaluate the difference in median serum protein level between responders and non-responders. Further, barplots of ACR20 and PASI75 response rate at week 16 for each treatment arm were generated for all subjects (protein level high and protein level low subjects, dichotomized according to median baseline level). Line plots of ACR20 and PASI75 response rate over time for each treatment arm were generated for protein level high and protein level low groups. Logistic regression models were also fit to evaluate associations between baseline protein levels and clinical outcome.

The median baseline serum levels of certain proteins in PsA patients were as listed below:
Median of β-defensin 2 (BD2): 9,265 ng/L
Median of IL-19:36 ng/L
Median of IL-17A: 0.575 ng/L
Median of C-reactive protein (CRP): 8.31 mg/L.

### Dichotomization

For certain analyses described herein, patients were dichotomized by median values (e.g., of the proteins listed above or other measures) into “high” and “low” groups, such that the high group had values above the median and the low group had values less than or equal to the median.

### β-Defensin 2 (BD2)

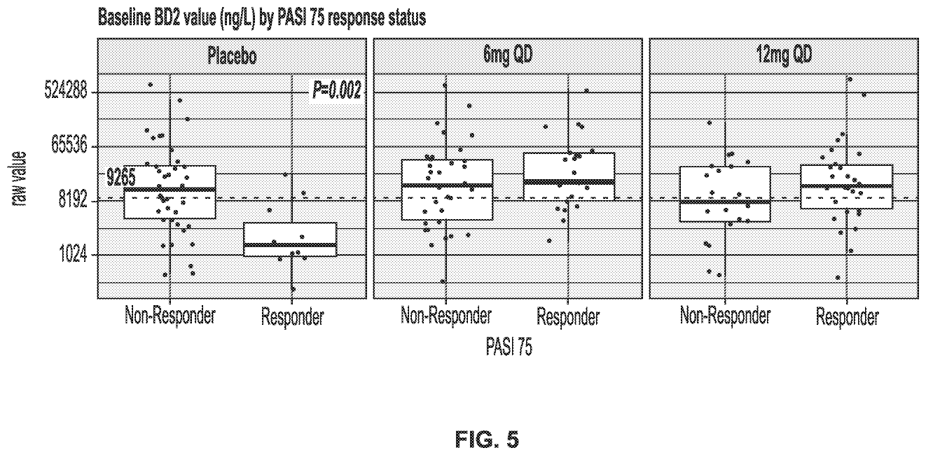

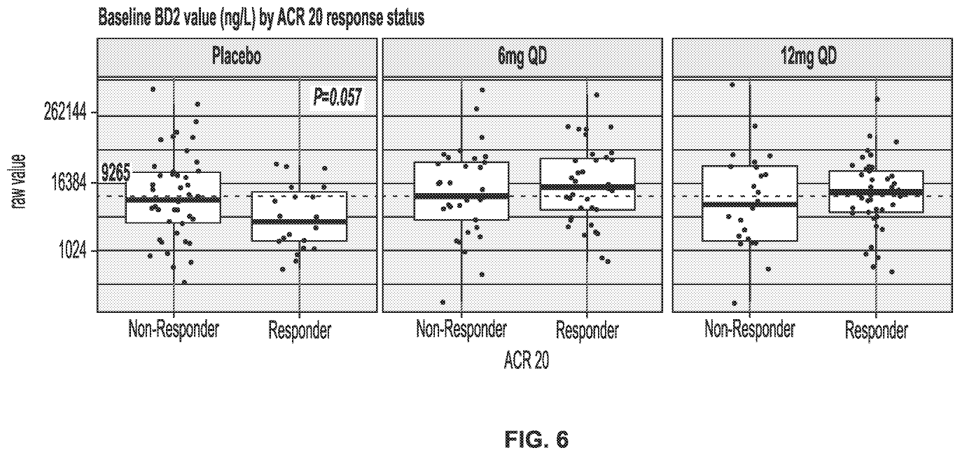

The median baseline level of β-defensin 2 (in all 200 subjects having baseline BD2 data) was 9,265 ng/L. FIG. 5 and FIG. 6 provide box plots of non-responders and responders (according to PASI75 response status and ACR20 response status, respectively) at week 16 in each treatment arm, with the dashed horizontal line corresponding to the median baseline BD2 level, and the solid horizontal line in each box representing the median baseline BD2 level for that box plot. Table 5 provides p-values for comparing baseline mean or baseline median BD2 level in responders versus non-responders, for each treatment arm; for the t-test to compare mean values, BD2 level was log 2 transformed prior to calculating the means for responders and non-responders.

TABLE 5
p-values for each treatment arm, comparing baseline
BD2 level in responders versus non-responders

| | t-test comparing mean | Wilcoxon rank sum test |
|---|---|---|
| | baseline BD2 level* in | comparing median baseline |
| | responders versus non- | BD2 level in responders |
| Treatment arm | responders | versus non-responders |
| | PASI75 responders versus non-responders | |
| Placebo | 0.00063 | 0.00191 |
| 6 mg QD | 0.23517 | 0.21124 |
| 12 mg QD | 0.16128 | 0.23162 |
| | ACR20 responders versus non-responders | |
| Placebo | 0.02295 | 0.05721 |
| 6 mg QD | 0.42895 | 0.48024 |
| 12 mg QD | 0.50135 | 0.47576 |

*log2 transformed

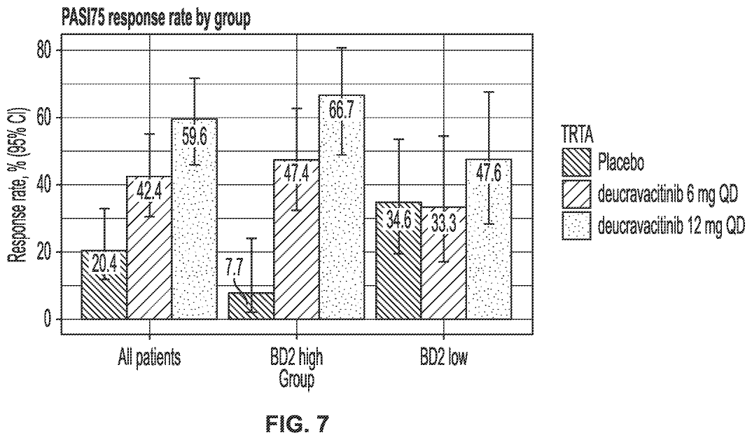

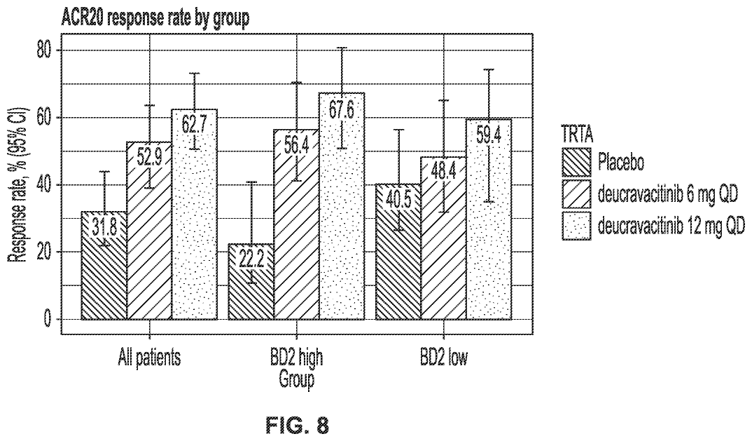

FIG. 7 and FIG. 8 are bargraphs showing the response rate in each treatment arm, for achieving PASI75 or ACR20 response status at week 16, for subjects with baseline BD2 levels that were greater than the median (“BD2 high”), or less than or equal to the median (“BD2 low”). For the BD2-high group, the PASI75 response rate was higher in both 6 mg QD and 12 mg QD treatment arms compared to the placebo arm, whereas in the BD2-low group, the PASI75 response rate differences between the treatment arms and the placebo arm were less pronounced. Similar results were observed for the ACR20 response rate.

Table 6 below provides the number of responders and non-responders in BD2-low and BD2-high groups, by treatment arm.

TABLE 6
Number of non-responders and responders in BD2-
low and BD2-high groups, by treatment arm

| | | | |
|---|---|---|---|
| | | Below/above | |
| | Response status at | median baseline | Number of |
| Treatment arm | week 16 | BD2 level | subjects |

PASI75 response status

| | | | |
|---|---|---|---|
| Placebo | Non-responder | Below | 22 |
| | | Above | 21 |
| | Responder | Below | 15 |
| | | Above | 6 |
| 6 mg QD | Non-responder | Below | 16 |
| | | Above | 17 |
| | Responder | Below | 15 |
| | | Above | 22 |
| 12 mg QD | Non-responder | Below | 13 |
| | | Above | 11 |
| | Responder | Below | 19 |
| | | Above | 23 |

ACR20 response status

| | | | |
|---|---|---|---|
| Placebo | Non-responder | Below | 17 |
| | | Above | 24 |
| | Responder | Below | 9 |
| | | Above | 2 |
| 6 mg QD | Non-responder | Below | 14 |
| | | Above | 20 |
| | Responder | Below | 7 |
| | | Above | 18 |
| 12 mg QD | Non-responder | Below | 11 |
| | | Above | 10 |
| | Responder | Below | 10 |
| | | Above | 20 |

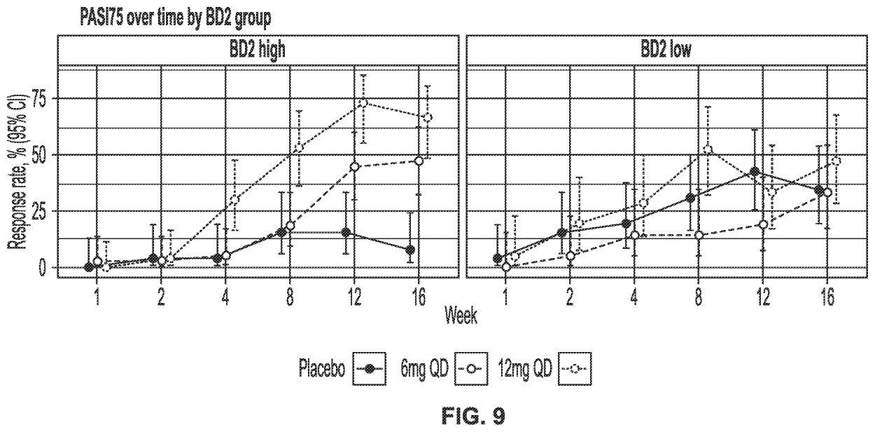

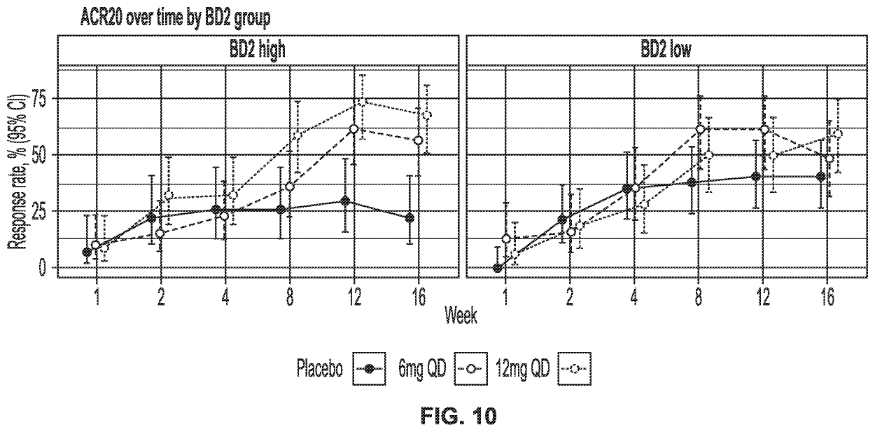

FIG. 9 and FIG. 10 provide line plots of response rate (PASI75 and ACR20, respectively) over time, for the BD2-high and the BD2-low groups. A dose-dependent PASI75 response rate over time was observed in the BD2-high group but not in the BD2-low group. In addition, higher ACR20 response rates to deucravacitinib over time, compared to the response rate in the placebo arm, were observed in the BD2-high group but not in the BD2-low group.

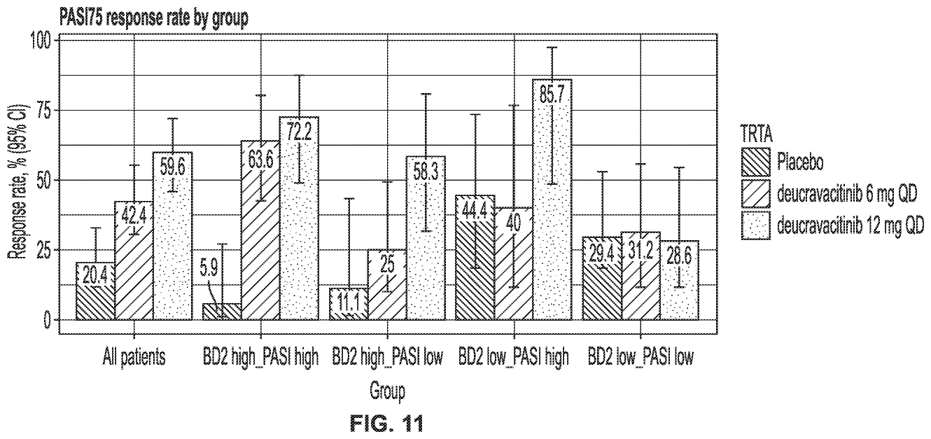

PsA patients were also dichotomized by their baseline BD2 level and baseline PASI status into four groups: BD2-high and PASI-high; BD2-high and PASI-low; BD2-low and PASI-high; and BD2-low and PASI-low. Table 7 provides the number of PsA patients within each of the four aforementioned groups. FIG. 11 is a bargraph of PASI75 response status at week 16 in each treatment arm for all subjects, as categorized into the aforementioned four groups. The bargraph shows that baseline BD2 level and baseline PASI are associated with PASI75 response. A high baseline BD2 level was associated with a better PASI75 response to deucravacitinib, in both the PASI-high and the PASI-low groups. These results indicate that a high baseline BD2 level is associated with greater clinical benefit of TYK2 inhibitor treatment as assessed by both ACR20 and PASI75.

TABLE 7
Number of PASI75 non-responders and responders in BD2-
high-PASI-high, BD2-high-PASI-low, BD2-low-PASI-high,
and BD2-low-PASI-low groups, by treatment arm

| Treatment | PASI75 | | Number of |
|---|---|---|---|
| arm | response status | Group | subjects |
| Placebo | Responder | BD2-high-PASI-high | 1 |
| | | BD2-high-PASI-low | 1 |
| | | BD2-low-PASI-high | 4 |
| | | BD2-low-PASI-low | 5 |
| | Non-responder | BD2-high-PASI-high | 16 |
| | | BD2-high-PASI-low | 8 |
| | | BD2-low-PASI-high | 5 |
| | | BD2-low-PASI-low | 12 |
| 6 mg QD | Responder | BD2-high-PASI-high | 14 |
| | | BD2-high-PASI-low | 4 |
| | | BD2-low-PASI-high | 2 |
| | | BD2-low-PASI-low | 5 |
| | Non-responder | BD2-high-PASI-high | 8 |
| | | BD2-high-PASI-low | 12 |
| | | BD2-low-PASI-high | 3 |
| | | BD2-low-PASI-low | 11 |
| 12 mg QD | Responder | BD2-high-PASI-high | 13 |
| | | BD2-high-PASI-low | 7 |
| | | BD2-low-PASI-high | 6 |
| | | BD2-low-PASI-low | 4 |
| | Non-responder | BD2-high-PASI-high | 5 |
| | | BD2-high-PASI-low | 5 |
| | | BD2-low-PASI-high | 1 |
| | | BD2-low-PASI-low | 10 |

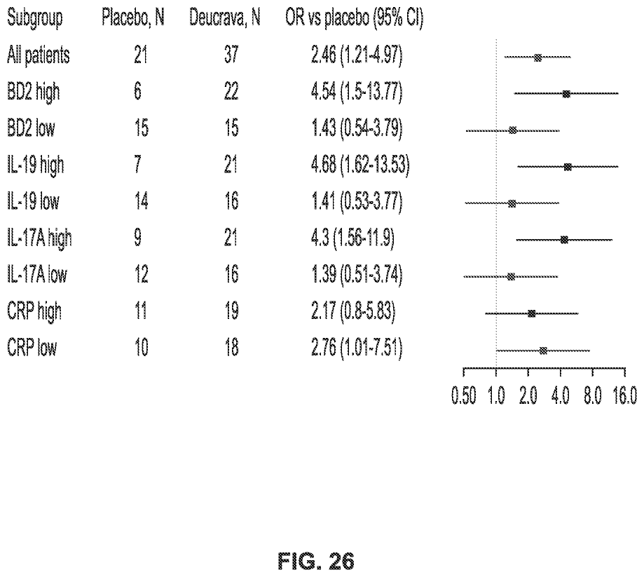

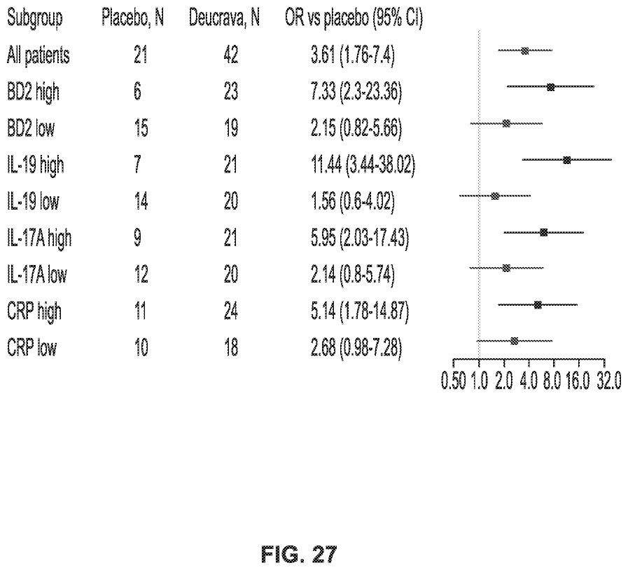

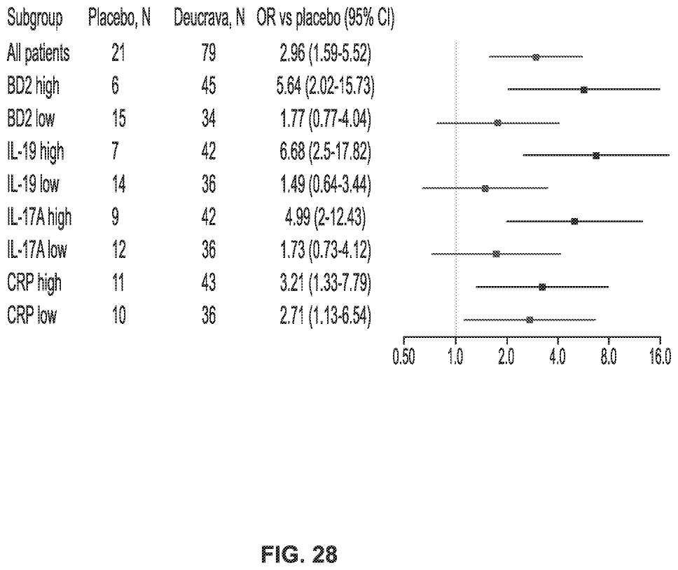

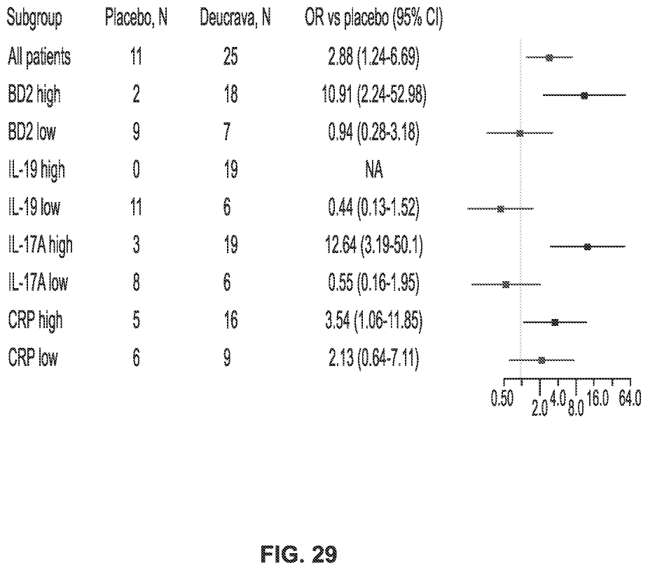

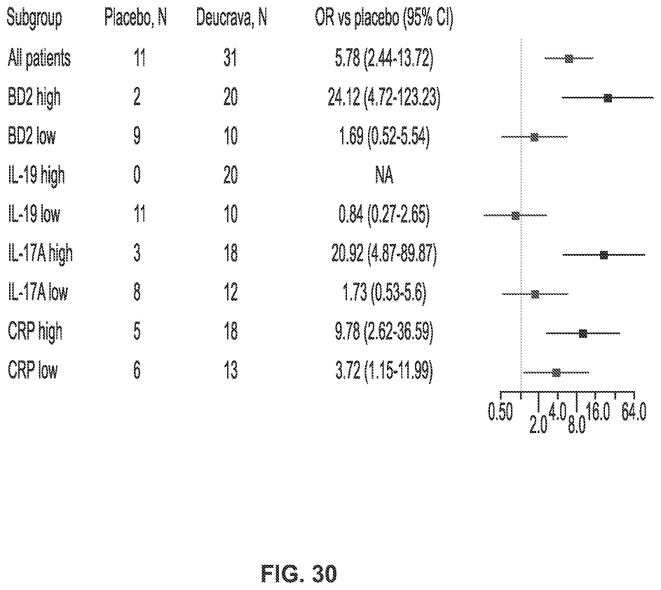

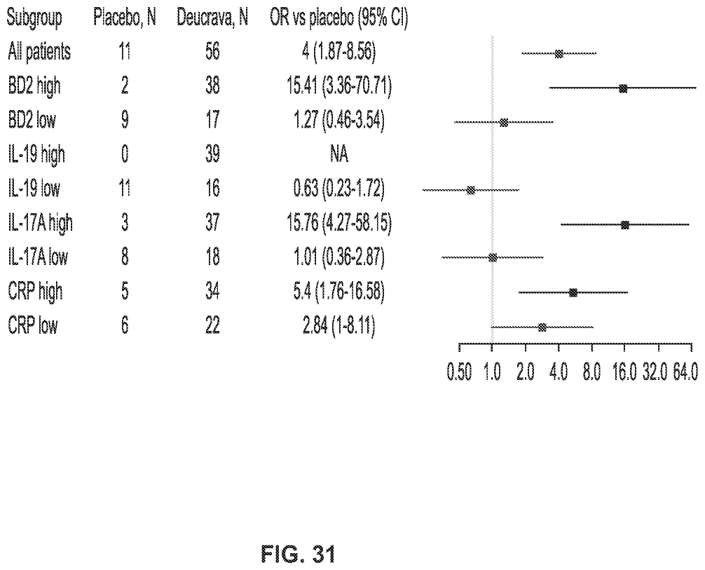

In addition, logistic regression models were fit for response measures ACR20 and PASI75, to generate the odds ratio of each of the deucravacitinib treatment arms versus placebo, in BD2-high and BD2-low groups. The following logistic regression models were used:
For ACR20 response status: ACR20 (Y/N)=treatment arm+BD2 group+treatment arm*BD2 group+TNF-inhibitor use+baseline weight (continuous).
For PASI75 response status: PASI75 (Y/N)=treatment arm+BD2 group+treatment arm*BD2 group+TNF-inhibitor use+baseline weight (continuous).
   The p-value of the interaction term was used to identify whether the OR for achieving the response was significantly different between BD2-high and BD2-low groups. FIGS. 26-31 are forest plots summarizing the logistic regression results of these models (for BD2 and other biomarkers described herein); each “N,” for “Placebo” and “Deucrava,” represents the number of responders in that group. The statistical model used to analyze the effect of baseline BD2 level was run with each treatment arm (6 mg QD, 12 mg QD, and placebo) as a separate predictor (see FIG. 26, FIG. 27, and Table 8A for ACR20 results; see FIG. 29, FIG. 30, and Table 9A for PASI75 results) and also with the deucravacitinib treatment arms combined (see FIG. 28 and Table 8B for ACR20 results; see FIG. 31 and Table 9B for PASI75 results).

TABLE 8A
Logistic regression odds ratio results for correlating
ACR20 response status with baseline BD2 level

| | | Odds | | | |
|---|---|---|---|---|---|
| | | Ratio or | | | |
| | | Interaction | | Confidence Interval | |
| Group | Comparison | Term | p-value | lower | upper |
| All | 6 mg QD vs | 2.46 | 0.012 | 1.214 | 4.96 |
| | placebo | | | | |
| All | 12 mg QD | 3.61 | <0.0001 | 1.759 | 7.41 |
| | vs placebo | | | | |
| BD2 high | 6 mg QD vs | 4.54 | 0.008 | 1.497 | 13.77 |
| | placebo | | | | |
| BD2 high | 12 mg QD | 7.33 | 0.001 | 2.301 | 23.36 |
| | vs placebo | | | | |
| BD2 low | 6 mg QD vs | 1.44 | 0.467 | 0.543 | 3.79 |
| | placebo | | | | |
| BD2 low | 12 mg QD | 2.15 | 0.120 | 0.819 | 5.66 |
| | vs placebo | | | | |
| Interaction | 6 mg QD vs | 3.16 | 0.125 | 0.725 | 13.80 |
| term: | placebo: | | | | |
| treatment | BD2 high | | | | |
| arm*BD2 | versus BD2 | | | | |
| group | low | | | | |
| Interaction | 12 mg QD | 3.40 | 0.111 | 0.753 | 15.39 |
| term: | vs placebo: | | | | |
| treatment | BD2 high | | | | |
| arm*BD2 | versus BD2 | | | | |
| group | low | | | | |

The first two rows in Table 8A present the odds ratio comparing the odds of responding to 6 mg or 12 mg deucravacitinib versus placebo, in all patients (BD2-high and BD2-low groups), using the following: ACR20 (Y/N)=treatment arm+TNF-inhibitor use+baseline weight (continuous). The odds ratios of the third through eighth rows represent results from the following: ACR20 (Y/N)=treatment arm+BD2 group+treatment arm*BD2 group+TNF-inhibitor use+baseline weight (continuous). The last two rows represent the interaction terms. For example, the seventh row provides the ratio of (a) the odds ratio of 6 mg QD versus placebo in the BD2-high group, and (b) the odds ratio of 6 mg QD versus placebo in the BD2-low group, i.e., for comparing ORs of 4.54 to 1.44. The ratio was higher than the null hypothesis (i.e., 1), but was not statistically significant.

Results shown in Table 8A, FIG. 26, and FIG. 27 indicate that the BD2-high group showed a significant treatment benefit (as indicated by significantly greater odds of achieving ACR20 with deucravacitinib treatment, compared to the odds of achieving ACR20 in the placebo arm) at both deucravacitinib doses, whereas the BD2-low group did not show a significant treatment benefit. Similarly, ACR20 results presented in FIG. 28 and Table 8B show that deucravacitinib provided a significant treatment benefit only in the BD2-high group (see second row of Table 8B).

TABLE 8B
Logistic regression odds ratio results for correlating
ACR20 response status with baseline BD2 level
(deucravacitinib treatment arms combined)

| | | Odds | | | |
|---|---|---|---|---|---|
| | | Ratio or | | | |
| | | Interaction | | Confidence Interval | |
| Group | Comparison | Term | p-value | lower | upper |
| All | Deucravacitinib | 2.961 | 0.0006 | 1.588 | 5.519 |
| | vs placebo | | | | |
| BD2 high | Deucravacitinib | 5.645 | 0.0009 | 2.025 | 15.735 |
| | vs placebo | | | | |
| BD2 low | Deucravacitinib | 1.767 | 0.178 | 0.772 | 4.041 |
| | vs placebo | | | | |
| Interaction | Deucravacitinib | 3.195 | 0.083 | 0.858 | 11.901 |
| term: | vs placebo: | | | | |
| treatment | BD2 high | | | | |
| arm * BD2 | versus BD2 | | | | |
| group | low | | | | |

TABLE 9A
Logistic regression odds ratio results for correlating
PASI75 response status with baseline BD2 level

| | | Odds | | | |
|---|---|---|---|---|---|
| | | Ratio or | | | |
| | | Interaction | | Confidence Interval | |
| Group | Comparison | Term | p-value | lower | upper |
| All | 6 mg QD vs | 2.880 | 0.014 | 1.240 | 6.69 |
| | placebo | | | | |
| All | 12 mg QD | 5.781 | <0.0001 | 2.436 | 13.72 |
| | vs placebo | | | | |
| BD2 high | 6 mg QD vs | 10.905 | 0.003 | 2.245 | 52.98 |
| | placebo | | | | |
| BD2 high | 12 mg QD | 24.118 | <0.0001 | 4.720 | 123.23 |
| | vs placebo | | | | |
| BD2 low | 6 mg QD vs | 0.938 | 0.918 | 0.277 | 3.18 |
| | placebo | | | | |
| BD2 low | 12 mg QD | 1.695 | 0.383 | 0.518 | 5.54 |
| | vs placebo | | | | |
| Interaction | 6 mg QD vs | 11.628 | 0.016 | 1.580 | 85.58 |
| term: | placebo: | | | | |
| treatment | BD2 high | | | | |
| arm*BD2 | versus BD2 | | | | |
| group | low | | | | |
| Interaction | 12 mg QD | 14.233 | 0.010 | 1.890 | 107.17 |
| term: | vs placebo: | | | | |
| treatment | BD2 high | | | | |
| arm*BD2 | versus BD2 | | | | |
| group | low | | | | |

FIGS. 29-31, Table 9A, and Table 9B provide the results of an analogous analysis for PASI75 response. Results shown in FIG. 29, FIG. 30, and Table 9A indicate that the BD2-high group showed a significant treatment benefit (as indicated by significantly greater odds of achieving PASI75 with deucravacitinib treatment, compared to the odds of achieving PASI75 in the placebo arm) at both deucravacitinib doses (see third and fourth rows), whereas the BD2-low group did not show a significant treatment benefit (see fifth and sixth rows). Furthermore, the treatment benefit (the odds ratio of showing a treatment response to deucravacitinib, versus the odds ratio of showing a response to placebo) at each dose was significantly higher in the BD2-high group than in the BD2-low group (see seventh and eighth rows).

The PASI75 response results presented in FIG. 31 and Table 9B indicate that deucravacitinib provided a significant treatment benefit only in the BD2-high group (see second row of Table 9B) and that the treatment benefit was significantly higher in the BD2-high group than in the BD2-low group (see the last row of Table 9B).

TABLE 9B
Logistic regression odds ratio results for correlating
PASI75 response status with baseline BD2 level
(deucravacitinib treatment arms combined)

| | | Odds | | | |
|---|---|---|---|---|---|
| | | Ratio or | | | |
| | | Interaction | | Confidence Interval | |
| Group | Comparison | Term | p-value | lower | upper |
| All | Deucravacitinib | 3.998 | 0.0004 | 1.866 | 8.563 |
| | vs placebo | | | | |
| BD2 high | Deucravacitinib | 15.415 | 0.0004 | 3.360 | 70.715 |
| | vs placebo | | | | |
| BD2 low | Deucravacitinib | 1.274 | 0.6427 | 0.458 | 3.543 |
| | vs placebo | | | | |
| Interaction | Deucravacitinib | 12.100 | 0.0078 | 1.929 | 75.898 |
| term: | vs placebo: | | | | |
| treatment | BD2 high | | | | |
| arm * BD2 | versus BD2 | | | | |
| group | low | | | | |

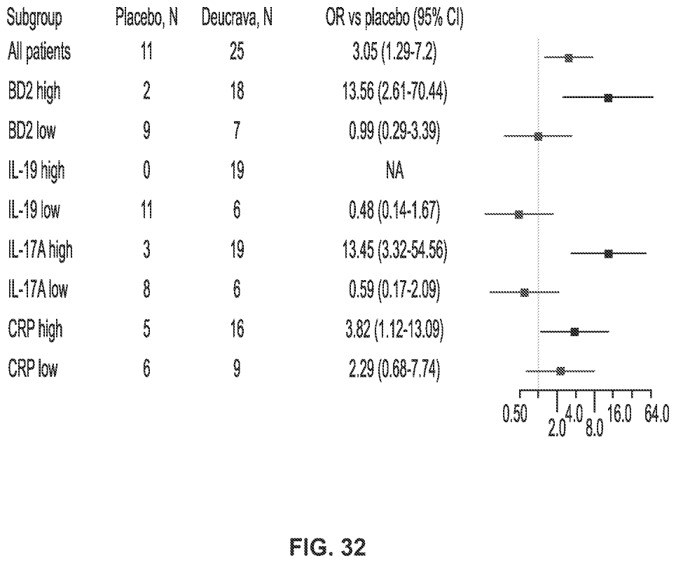

The above logistic regression model did not adjust for baseline disease activity. To account for any potential confounding effects of baseline disease activity, a second model that adjusted for baseline disease activity was used. FIGS. 32-35 provide forest plots summarizing the results of this second model; Tables 10 and 11 provide the odds ratio results for BD2 in this model.

TABLE 10
Logistic regression odds ratio results for correlating ACR20 response
status with baseline BD2 level, adjusted for baseline DAS28

| | | Odds | | | |
|---|---|---|---|---|---|
| | | Ratio or | | | |
| | | Interaction | | Confidence Interval | |
| Group | Comparison | Term | p-value | lower | upper |
| All | 6 mg QD vs | 2.44 | 0.013 | 1.207 | 4.94 |
| | placebo | | | | |
| All | 12 mg QD | 3.62 | <0.0001 | 1.763 | 7.48 |
| | vs placebo | | | | |
| BD2-high | 6 mg QD vs | 4.42 | 0.009 | 1.450 | 13.46 |
| | placebo | | | | |
| BD2-high | 12 mg QD | 7.33 | 0.001 | 2.299 | 23.39 |
| | vs placebo | | | | |
| BD2-low | 6 mg QD vs | 1.44 | 0.461 | 0.545 | 3.81 |
| | placebo | | | | |
| BD2-low | 12 mg QD | 2.14 | 0.122 | 0.815 | 5.64 |
| | vs placebo | | | | |
| Interaction | 6 mg QD vs | 3.06 | 0.138 | 0.699 | 13.43 |
| term: | placebo: | | | | |
| treatment | BD2-high | | | | |
| arm*BD2 | versus BD2- | | | | |
| group | low | | | | |
| Interaction | 12 mg QD | 3.42 | 0.110 | 0.756 | 15.48 |
| term: | vs placebo: | | | | |
| treatment | BD2-high | | | | |
| arm*BD2 | versus BD2- | | | | |
| group | low | | | | |

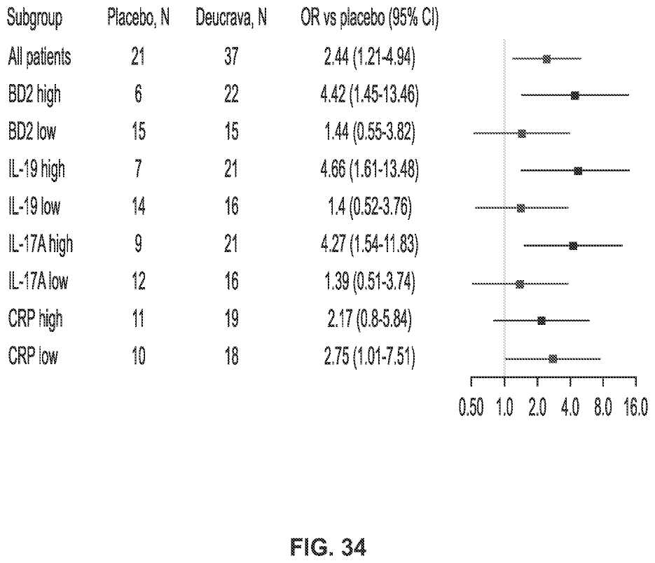

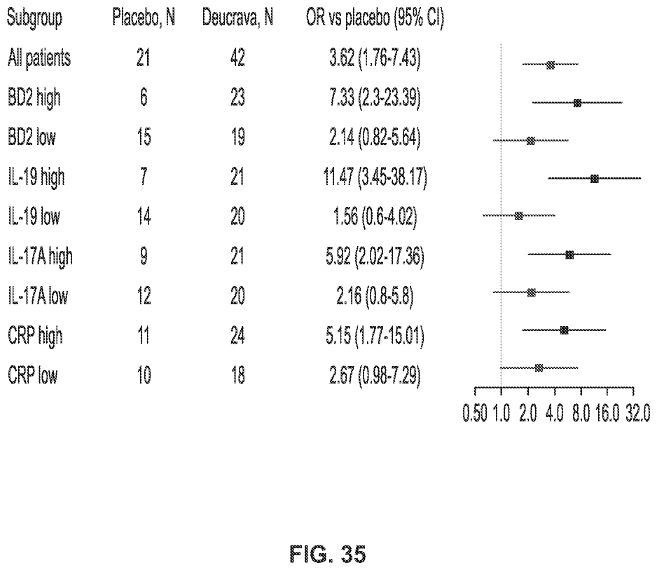

FIG. 34, FIG. 35, and Table 10 present the results of the second model for ACR20 response status; the table provides the odds ratio results for ACR20 response status at week 16, adjusted for baseline DAS28. The first two rows in Table 10 provide the odds ratio and p-value for achieving ACR20 response, when adjusted for baseline DAS28, for all patients (BD2-high and BD2-low) using the following: ACR20 (Y/N)=treatment arm+TNF-inhibitor use+baseline weight (continuous)+baseline DAS28 (continuous). The odds ratios represent the odds ratios comparing 6 mg or 12 mg versus placebo. These results show that the odds of achieving an ACR20 response was significantly higher in the deucravacitinib treatment arms than in the placebo arm, even after adjusting for baseline DAS28.

The results provided in the third through sixth rows in Table 10 were obtained using the following: ACR20 (Y/N)=treatment arm*BD2 group+TNF-inhibitor use+baseline weight (continuous)+baseline DAS28 (continuous). The third and fourth rows provide odds ratio results for the BD2-high groups. These results indicate that for the BD2-high patients, after adjusting for baseline DAS28, the odds of achieving ACR20 response in each of the deucravacitinib treatment arms was significantly greater than the odds of achieving ACR20 response in the placebo arm. In contrast, such significant odds ratio results were not observed for the BD2-low groups (see fifth and sixth rows in Table 10). These results indicate that a high baseline BD2 level was associated with significant clinical benefit of TYK2 inhibitor therapy (as indicated by significantly greater odds of achieving ACR20 with TYK2 inhibitor treatment, compared to the odds of achieving ACR20 with placebo), independent of baseline DAS28.

The results provided in the seventh and eighth rows in Table 10 were obtained using the same model that was used for the third through sixth rows. These results from the interaction terms were not statistically significant but are consistent with other results and show that the ratio of (a) the odds ratio for achieving ACR20 response with deucravacitinib treatment versus placebo in the BD2-high group, to (b) the odds ratio for achieving ACR20 response with deucravacitinib treatment versus placebo in the BD2-low group, was numerically higher than the null hypothesis (i.e., 1). In other words, the odds ratio of responding (as measured by ACR20) to deucravacitinib treatment, versus the odds ratio of responding to placebo, was higher in the BD2-high group than in the BD2-low group.

TABLE 11
Logistic regression odds ratio results for correlating PASI75 response
status with baseline BD2 level, adjusted for baseline PASI score

| | | Odds | | | |
|---|---|---|---|---|---|
| | | Ratio or | | | |
| | | Interaction | | Confidence Interval | |
| Group | Comparison | Term | p-value | lower | upper |
| All | 6 mg QD vs | 3.052 | 0.011 | 1.293 | 7.20 |
| | placebo | | | | |
| All | 12 mg QD | 6.494 | <0.0001 | 2.665 | 15.82 |
| | vs placebo | | | | |
| BD2-high | 6 mg QD vs | 13.559 | 0.002 | 2.610 | 70.44 |
| | placebo | | | | |
| BD2-high | 12 mg QD | 34.074 | <0.0001 | 6.073 | 191.19 |
| | vs placebo | | | | |
| BD2-low | 6 mg QD vs | 0.987 | 0.983 | 0.287 | 3.39 |
| | placebo | | | | |
| BD2-low | 12 mg QD | 1.751 | 0.359 | 0.529 | 5.80 |
| | vs placebo | | | | |
| Interaction | 6 mg QD vs | 13.742 | 0.012 | 1.768 | 106.81 |
| term: | placebo: | | | | |
| treatment | BD2-high | | | | |
| arm*BD2 | versus BD2- | | | | |
| group | low | | | | |
| Interaction | 12 mg QD | 19.455 | 0.006 | 2.392 | 158.21 |
| term: | vs placebo: | | | | |
| treatment | BD2-high | | | | |
| arm*BD2 | versus BD2- | | | | |
| group | low | | | | |

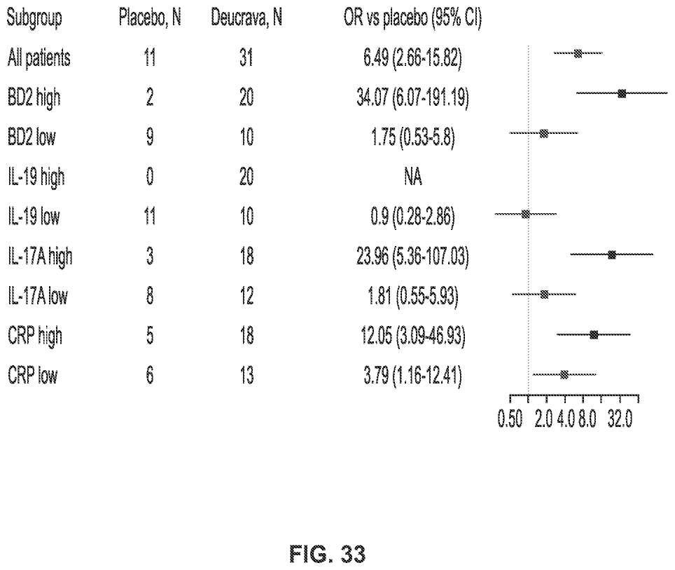

FIG. 32, FIG. 33, and Table 11 present the results of an analogous model for PASI75 response status; the table provides the odds ratio results for PASI75 status at week 16, adjusted for baseline PASI score. The first two rows in Table 11 provide the odds ratio and p-value for achieving PASI75 response, when adjusted for baseline PASI score, for all patients (BD2-high and BD2-low) using the following: PASI75 (Y/N)=treatment arm+TNF-inhibitor use+baseline weight (continuous)+baseline PASI score (continuous). The odds ratios represent the odds ratios comparing 6 mg or 12 mg versus placebo. These results show that the odds of achieving a PASI75 response was significantly higher in the deucravacitinib treatment arms than in the placebo arm, even after adjusting for baseline PASI score.

The results provided in the third through sixth rows of Table 11 were obtained using the following: PASI75 (Y/N)=treatment arm*BD2 group+TNF-inhibitor use+baseline weight (continuous)+baseline PASI score (continuous). The third and fourth rows provide odds ratio results for the BD2-high groups. For the BD2-high patients, after adjusting for baseline PASI score, the odds of achieving PASI75 response in each deucravacitinib treatment arm was significantly greater than the odds of achieving PASI75 in the placebo arm. In contrast, such significant odds ratio results were not observed in the BD2-low groups (see fifth and sixth rows in Table 11). These results indicate that a high baseline BD2 level was associated with significant clinical benefit of TYK2 inhibitor treatment (as indicated by significantly greater odds of achieving PASI75 with TYK2 inhibitor treatment, compared to the odds of achieving PASI75 in the placebo arm), independent of baseline PASI score.

The results provided in the seventh and eighth rows in Table 11 were obtained using the same model that was used for the third through sixth rows. The ratio of (a) the odds ratio for achieving PASI75 after treatment with deucravacitinib versus placebo in the BD2-high group to (b) the odds ratio for achieving PASI75 after treatment with deucravacitinib versus placebo in the BD2-low group, was statistically significantly higher than the null hypothesis (i.e., 1). These results show that, compared with a low BD2 level, a high BD2 level was associated with a significantly greater clinical benefit (indicated by significantly greater odds ratio for achieving PASI75) of TYK2 inhibitor treatment compared with placebo, even after adjusting for baseline PASI.

For PASI75, an additional logistic regression model was fit to assess the prediction value of baseline BD2 level and baseline PASI score, each dichotomized according to median value. This model generated the odds ratio of each of the dosed arms versus placebo, for each of the four baseline BD2-PASI groups (i.e., BD2-high-PASI-high; BD2-high-PASI-low; BD2-low-PASI-high; BD2-low-PASI-low; see FIG. 11). This model was: PASI75 (Y/N)=treatment arm+baseline BD2-PASI group+treatment arm*baseline BD2-PASI group+TNF-inhibitor use+baseline weight (continuous). Table 12 presents the results of this model.

TABLE 12
Logistic regression odds ratio results for correlating PASI75
response status with baseline BD2 level and baseline PASI

| | | | | Confidence Interval | |
|---|---|---|---|---|---|
| Group | Comparison | Odds Ratio | p-value | lower | upper |
| All | 6 mg QD vs | 2.880 | 0.014 | 1.240 | 6.689 |
| | placebo | | | | |
| All | 12 mg QD vs | 5.781 | <0.0001 | 2.436 | 13.720 |
| | placebo | | | | |
| BD2-high- | 6 mg QD vs | 29.670 | 0.003 | 3.247 | 271.150 |
| PASI- | placebo | | | | |
| high | | | | | |
| BD2-high- | 12 mg QD vs | 43.084 | 0.001 | 4.416 | 420.380 |
| PASI- | placebo | | | | |
| high | | | | | |
| BD2-high- | 6 mg QD vs | 2.331 | 0.489 | 0.212 | 25.599 |
| PASI-low | placebo | | | | |
| BD2-high- | 12 mg QD vs | 10.995 | 0.048 | 1.019 | 118.576 |
| PASI-low | placebo | | | | |
| BD2-low- | 6 mg QD vs | 0.825 | 0.867 | 0.087 | 7.851 |
| PASI- | placebo | | | | |
| high | | | | | |
| BD2-low- | 12 mg QD vs | 7.787 | 0.112 | 0.621 | 97.599 |
| PASI- | placebo | | | | |
| high | | | | | |
| BD2-low- | 6 mg QD vs | 1.093 | 0.907 | 0.247 | 4.838 |
| PASI-low | placebo | | | | |
| BD2-low- | 12 mg QD vs | 0.967 | 0.966 | 0.203 | 4.610 |
| PASI-low | placebo | | | | |

In Table 12, the first two rows are the same as the first two rows in Table 9 and provide the odds ratios of comparing 6 mg QD or 12 mg QD to placebo, in all patients, using the following: PASI75 (Y/N)=treatment arm+TNF-inhibitor use+baseline weight (continuous). The odds ratios of the third through tenth rows provide results from the following: PASI75 (Y/N)=treatment arm+baseline BD2-PASI group+treatment arm*baseline BD2-PASI group+TNF-inhibitor use+baseline weight (continuous). For example, in the third row, 29.67 is the odds ratio of achieving a response in the 6 mg QD treatment arm versus the placebo arm for patients with a BD2 level above median and a baseline PASI score above median.

The results in Table 12 indicate that in both deucravacitinib treatment arms, the BD2-high-PASI-high groups showed a significantly greater odds of responding (as indicated by PASI75) to deucravacitinib, compared with the odds of responding to placebo (see third and fourth rows), whereas the BD2-low-PASI-high groups did not (see seventh and eighth rows). Similar significant treatment benefit was observed for the BD2-high-PASI-low group in the 12 mg QD treatment arm compared with placebo.

### IL-19

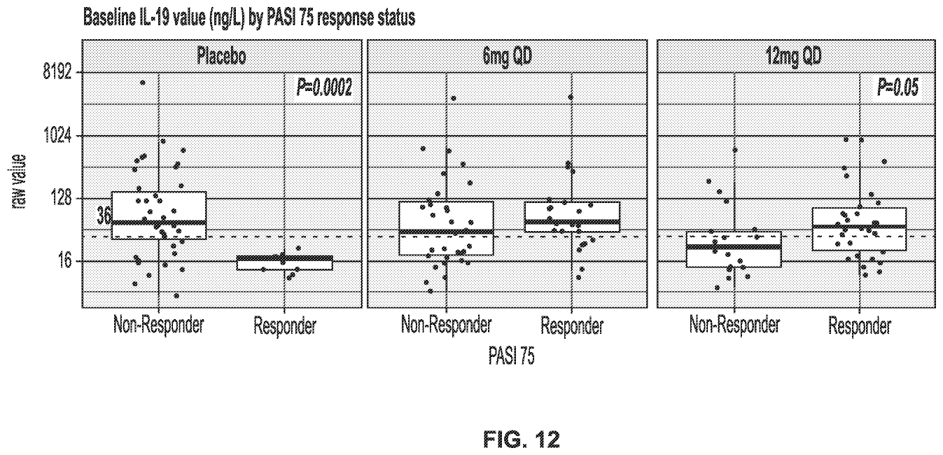

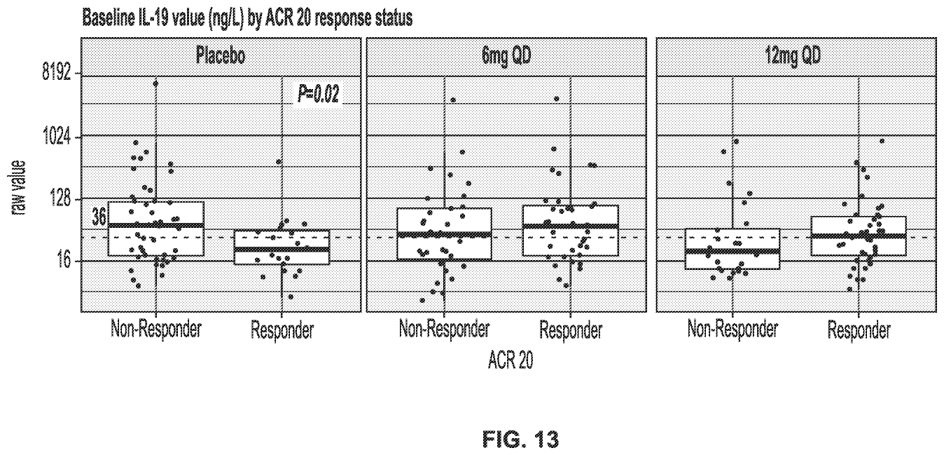

The same analyses were performed for baseline IL-19 levels measured in PsA patients. The median baseline level of IL-19 (for all 201 subjects having IL-19 baseline data) was 36 ng/L. FIG. 12 and FIG. 13 provide box plots of non-responders and responders (according to PASI75 response status and ACR20 response status, respectively) at week 16 in each treatment arm, with the dashed horizontal line corresponding to the median baseline IL-19 level, and the solid horizontal line in each box representing the median baseline IL-19 level for that box plot. Table 13 provides p-values for comparing baseline mean or baseline median IL-19 level in responders versus non-responders, for each treatment arm; for the t-test to compare mean values, IL-19 levels were log 2 transformed prior to calculating the means for responders and non-responders.

TABLE 13
p-values for each treatment arm, comparing baseline
IL-19 level in responders versus non-responders

| | t-test comparing mean | Wilcoxon rank sum test |
|---|---|---|
| | baseline IL-19 level* in | comparing median baseline |
| | responders versus non- | IL-19 level in responders |
| Treatment arm | responders | versus non-responders |
| | PASI75 responders versus non-responders | |
| Placebo | <0.0001 | 0.00022 |
| 6 mg QD | 0.29995 | 0.19753 |
| 12 mg QD | 0.08646 | 0.04762 |
| | ACR20 responders versus non-responders | |
| Placebo | 0.00525 | 0.01627 |
| 6 mg QD | 0.44843 | 0.39362 |
| 12 mg QD | 0.47532 | 0.1761 |

*log2 transformed

As shown in FIG. 12, for the placebo group, higher baseline IL-19 levels were observed in PASI75 non-responders compared to PASI75 responders. In contrast, for the deucravacitinib treatment groups, baseline IL-19 levels were higher in PASI75 responders compared to PASI75 non-responders. These results indicate that PsA patients with high baseline IL-19 levels had a better skin response to deucravacitinib compared with patients with low baseline IL-19 levels.

In addition, as shown in FIG. 13, in the placebo group, higher baseline IL-19 levels were observed in ACR20 non-responders compared to ACR20 responders. In contrast, responders in the deucravacitinib treated groups had higher baseline IL-19 levels compared to the non-responders. These results indicate that PsA patients with high baseline IL-19 levels had a better joint response to deucravacitinib compared with patients with low baseline IL-19 levels.

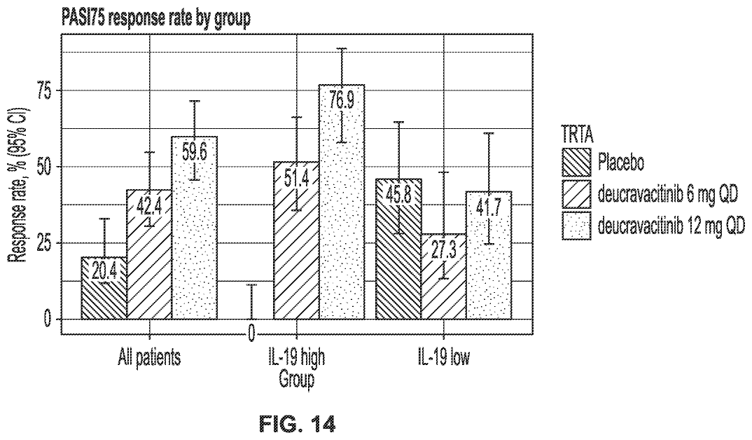

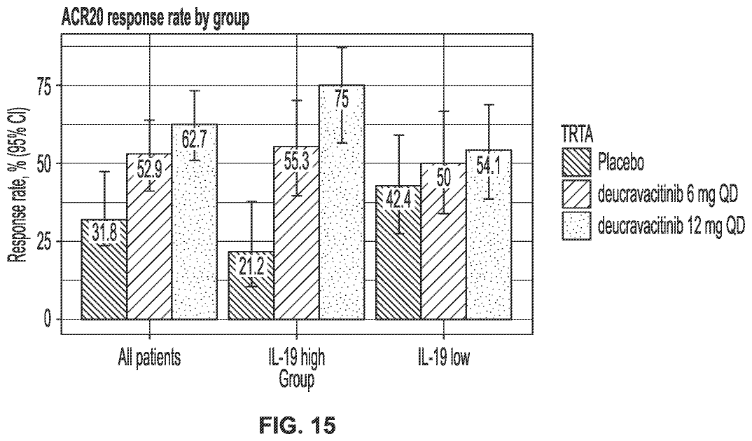

FIG. 14 and FIG. 15 are bargraphs showing the response rate in each treatment arm, for achieving PASI75 or ACR20 response status at week 16, for subjects in IL-19-high and IL-19-low groups (dichotomized according to median IL-19 baseline level). For the IL-19-high group, the PASI75 response rate was higher in both 6 mg QD and 12 mg QD treatment arms compared to the response rate in the placebo arm; this result was not observed for the IL-19-low-group (see FIG. 14). For the ACR20 response rate, a much more pronounced treatment effect (indicating a dose response effect of deucravacitinib) was observed in the IL-19-high group than in the IL-19-low group (see FIG. 15).

Table 14 below provides the number of responders and non-responders in IL-19-low and IL-19-high groups (classified according to IL-19 median level in PsA patients), by treatment arm.

TABLE 14
Number of non-responders and responders in IL-
19-low and IL-19-high groups, by treatment arm

| | | | |
|---|---|---|---|
| | Response | Below/above | |
| | status at | median baseline | Number of |
| Treatment arm | week 16 | IL-19 level | subjects |

PASI75 response status

| | | | |
|---|---|---|---|
| Placebo | Non-responder | Below | 13 |
| | | Above | 30 |
| | Responder | Below | 11 |
| | | Above | 0 |
| 6 mg QD | Non-responder | Below | 16 |
| | | Above | 18 |
| | Responder | Below | 6 |
| | | Above | 19 |
| 12 mg QD | Non-responder | Below | 14 |
| | | Above | 6 |
| | Responder | Below | 10 |
| | | Above | 20 |

ACR20 response status

| | | | |
|---|---|---|---|
| Placebo | Non-responder | Below | 19 |
| | | Above | 26 |
| | Responder | Below | 14 |
| | | Above | 7 |
| 6 mg QD | Non-responder | Below | 16 |
| | | Above | 17 |
| | Responder | Below | 16 |
| | | Above | 21 |
| 12 mg QD | Non-responder | Below | 17 |
| | | Above | 7 |
| | Responder | Below | 20 |
| | | Above | 21 |

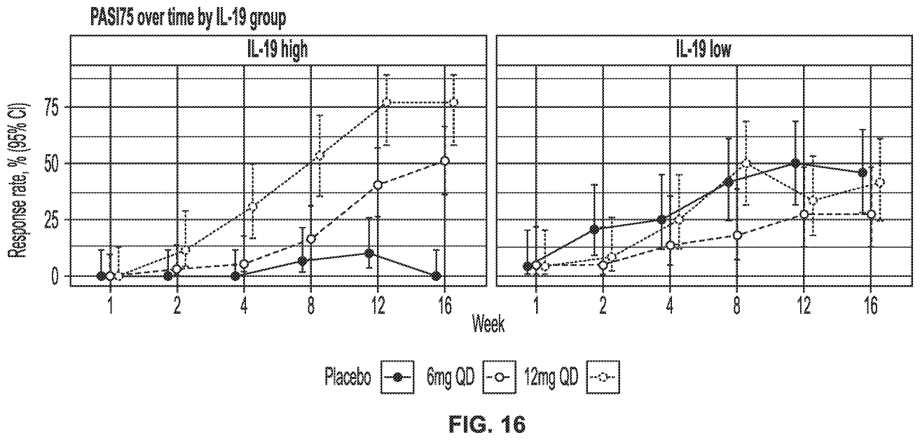

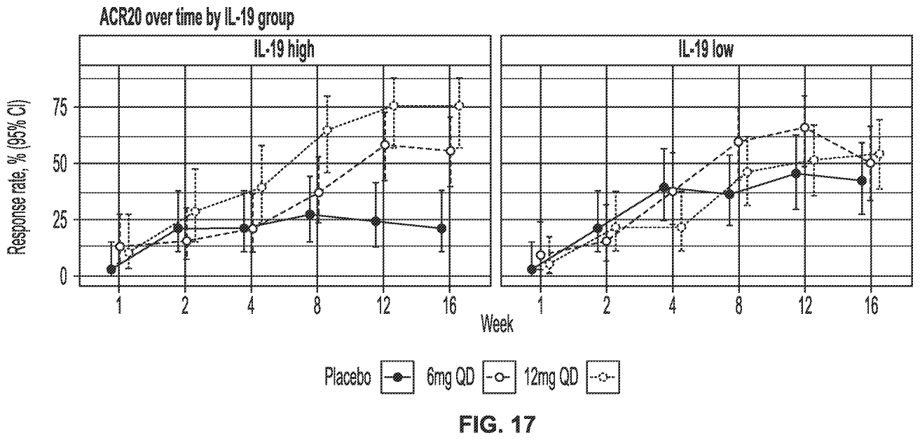

FIG. 16 and FIG. 17 provide line plots of response rate (PASI75 and ACR20, respectively) over time, for IL-19 baseline high and IL-19 baseline low groups. A dose-dependent PASI75 response over time was observed in IL-19-high but not in IL-19-low PsA patients. In addition, higher ACR20 response rates to deucravacitinib over time compared to placebo were observed in IL-19-high but not in IL-19-low groups.

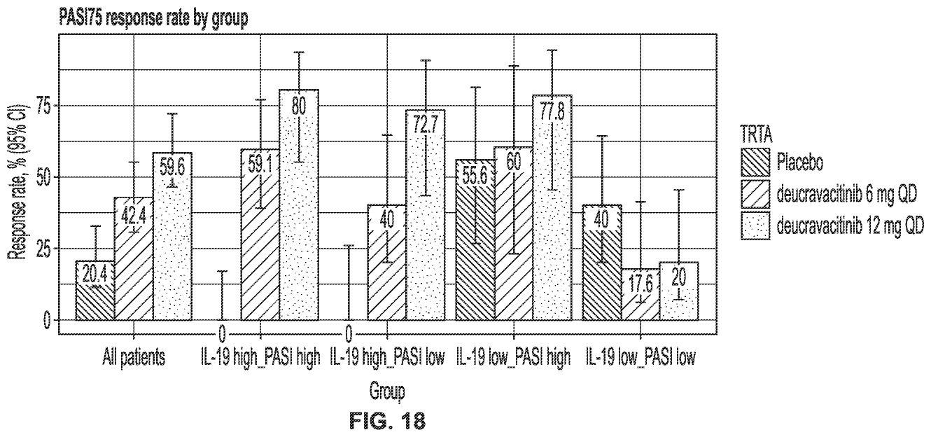

PsA patients also were categorized by their baseline IL-19 level (above or below median) and baseline PASI status (above or below median) into four groups: IL-19-high and PASI-high; IL-19-high and PASI-low; IL-19-low and PASI-high; and IL-19-low and PASI-low. Table 15 below provides the number of patients in each of the aforementioned groups. FIG. 18 is a barplot of PASI75 response status at week 16 in each treatment arm for all subjects, as categorized into the aforementioned four groups. The barplot shows that baseline IL-19 level is associated with PASI75 response, even for patients in the low-PASI group (see “IL-19-high PASI-low” group in FIG. 18).

TABLE 15
Number of PASI75 non-responders and responders in IL-
19-high-PASI-high, IL-19-high-PASI-low, IL-19-low-PASI-
high, and IL-19-low-PASI-low groups, by treatment arm

| Treatment | PASI75 | | Number of |
|---|---|---|---|
| arm | response status | Group | subjects |
| Placebo | Responder | IL-19-high-PASI-high | 0 |
| | | IL-19-high-PASI-low | 0 |
| | | IL-19-low-PASI-high | 5 |
| | | IL-19-low-PASI-low | 6 |
| | Non-responder | IL-19-high-PASI-high | 19 |
| | | IL-19-high-PASI-low | 11 |
| | | IL-19-low-PASI-high | 4 |
| | | IL-19-low-PASI-low | 9 |
| 6 mg QD | Responder | IL-19-high-PASI-high | 13 |
| | | IL-19-high-PASI-low | 6 |
| | | IL-19-low-PASI-high | 3 |
| | | IL-19-low-PASI-low | 3 |
| | Non-responder | IL-19-high-PASI-high | 9 |
| | | IL-19-high-PASI-low | 9 |
| | | IL-19-low-PASI-high | 2 |
| | | IL-19-low-PASI-low | 14 |
| 12 mg QD | Responder | IL-19-high-PASI-high | 12 |
| | | IL-19-high-PASI-low | 8 |
| | | IL-19-low-PASI-high | 7 |
| | | IL-19-low-PASI-low | 3 |
| | Non-responder | IL-19-high-PASI-high | 3 |
| | | IL-19-high-PASI-low | 3 |
| | | IL-19-low-PASI-high | 2 |
| | | IL-19-low-PASI-low | 12 |

Logistic regression models were fit as described above for BD2. For response measure ACR20, the odds ratio for each of the dosed arms versus placebo, in IL-19-high and IL-19-low groups, was generated. The model for ACR20 was: ACR20 (Y/N)=treatment arm+IL-19 group+treatment arm*IL-19 group+TNF-inhibitor use+baseline weight (continuous). The p-value of the interaction term was used to identify whether the OR for achieving ACR20 response (in the treatment arms versus placebo) was significantly different between IL-19-high and IL-19-low groups. The same regression model was fit for PASI75 response status: PASI75 (Y/N)=treatment arm+IL-19 group+treatment arm*IL-19 group+TNF-inhibitor use+baseline weight (continuous). For the logistic regression model results for PASI75 (see Table 17), odds ratios for the IL-19-high groups could not be calculated because there were no PASI75 responders in the IL-19-high placebo group (see Table 15). The interaction terms, for assessing the differences between the odds ratios of the IL-19-high groups and IL-19-low groups, also could not be calculated for PASI75 response. FIGS. 26-31 are forest plots summarizing the logistic regression results of these models (for IL-19 and other biomarkers described herein). The statistical model used to analyze the effect of baseline IL-19 level was run with each treatment arm (6 mg QD, 12 mg QD, and placebo) as a separate predictor (see FIG. 26, FIG. 27, and Table 16A for ACR20 results; see FIG. 29, FIG. 30, and Table 17A for PASI75 results) and also with the deucravacitinib treatment arms combined (see FIG. 28 and Table 16B for ACR20 results; see FIG. 31 and Table 17B for PASI75 results).

TABLE 16A
Logistic regression odds ratio results for correlating
ACR20 response status with baseline IL-19 level

| | | Odds | | | |
|---|---|---|---|---|---|
| | | Ratio or | | | |
| | | Interaction | | Confidence Interval | |
| Group | Comparison | Term | p-value | lower | upper |
| All | 6 mg QD vs | 2.46 | 0.012 | 1.214 | 4.96 |
| | placebo | | | | |
| All | 12 mg QD | 3.61 | <0.0001 | 1.759 | 7.41 |
| | vs placebo | | | | |
| IL-19 high | 6 mg QD vs | 4.68 | 0.004 | 1.621 | 13.53 |
| | placebo | | | | |
| IL-19 high | 12 mg QD | 11.44 | <0.0001 | 3.441 | 38.02 |
| | vs placebo | | | | |
| IL-19 low | 6 mg QD vs | 1.41 | 0.496 | 0.526 | 3.77 |
| | placebo | | | | |
| IL-19 low | 12 mg QD | 1.56 | 0.361 | 0.602 | 4.02 |
| | vs placebo | | | | |
| Interaction | 6 mg QD vs | 3.33 | 0.103 | 0.785 | 14.11 |
| term: | placebo: | | | | |
| treatment | IL-19 high | | | | |
| arm*IL-19 | versus IL-19 | | | | |
| group | low | | | | |
| Interaction | 12 mg QD | 7.35 | 0.011 | 1.587 | 34.02 |
| term: | vs placebo: | | | | |
| treatment | IL-19 high | | | | |
| arm*IL-19 | versus IL-19 | | | | |
| group | low | | | | |

The results shown in FIG. 26, FIG. 27, and Table 16A show that the odds of responding to deucravacitinib (achieving ACR20 response) in each treatment arm was statistically significantly higher than the odds of achieving ACR20 response in the placebo arm for the IL-19-high group, whereas no such result was observed for the IL-19-low group. Also, the treatment benefit (as indicated by the odds ratio of achieving ACR20 in response to deucravacitinib versus placebo) was greater in the IL-19-high group than in the IL-19-low group (numerically greater in the 6 mg QD arm and statistically significantly greater in the 12 mg QD arm).

TABLE 16B
Logistic regression odds ratio results for correlating
ACR20 response status with baseline IL-19 level
(deucravacitinib treatment arms combined)

| | | Odds | | | |
|---|---|---|---|---|---|
| | | Ratio or | | | |
| | | Interaction | | Confidence Interval | |
| Group | Comparison | Term | p-value | lower | upper |
| All | Deucravacitinib | 2.961 | 0.0006 | 1.588 | 5.519 |
| | vs placebo | | | | |
| IL-19 high | Deucravacitinib | 6.679 | 0.0001 | 2.502 | 17.825 |
| | vs placebo | | | | |
| IL-19 low | Deucravacitinib | 1.487 | 0.354 | 0.642 | 3.441 |
| | vs placebo | | | | |
| Interaction | Deucravacitinib | 4.491 | 0.022 | 1.236 | 16.316 |
| term: | vs placebo: IL- | | | | |
| treatment | 19 high versus | | | | |
| arm * IL- | IL-19 low | | | | |
| 19 group | | | | | |

Results presented in FIG. 28 and Table 16B show that deucravacitinib provided a significant treatment benefit in the IL-19 high group (see row two of Table 16B) and that the treatment benefit was significantly greater in the IL-19 high group compared with the IL-19 low group (see last row of Table 16B).

TABLE 17A
Logistic regression odds ratio results for correlating
PASI75 response status with baseline IL-19 level

| | | Odds | | | |
|---|---|---|---|---|---|
| | | Ratio | | Confidence Interval | |
| Group | Comparison | (OR) | p-value | lower | upper |
| All | 6 mg QD vs | 2.880 | 0.014 | 1.240 | 6.69 |
| | placebo | | | | |
| All | 12 mg QD | 5.781 | <0.0001 | 2.436 | 13.72 |
| | vs placebo | | | | |
| IL-19 low | 6 mg QD vs | 0.443 | 0.197 | 0.129 | 1.52 |
| | placebo | | | | |
| IL-19 low | 12 mg QD | 0.842 | 0.769 | 0.268 | 2.65 |
| | vs placebo | | | | |

TABLE 17B
Logistic regression odds ratio results for correlating
PASI75 response status with baseline IL-19 level
(deucravacitinib treatment arms combined)

| | | Odds | | | |
|---|---|---|---|---|---|
| | | Ratio or | | | |
| | | Interaction | | Confidence Interval | |
| Group¹ | Comparison | Term | p-value | lower | upper |
| All | Deucravacitinib | 3.998 | 0.0004 | 1.866 | 8.563 |
| | vs placebo | | | | |
| IL-19 low | Deucravacitinib | 6.284 | 0.367 | 0.229 | 1.725 |
| | vs placebo | | | | |

¹Because all subjects in the placebo arm were IL-19 low, it was not possible to determine the PASI75 treatment benefit in the IL-19 high group or whether it differed significantly between the IL-19 low group and the IL-19 high group.

The results presented FIGS. 29-31, Table 17A, and Table 17B show that there was a significant overall benefit of deucravacitinib in achieving PASI75. In the IL-19 low group, the treatment benefit did not reach statistical significance (see third and fourth rows of Table 17A and second row of Table 17B).

Additional logistic regression models were fit for baseline IL-19 expression and response status, as described above for BD2. Table 18 and Table 19 provide the results of the second model, which accounts for baseline disease activity, as described further below.

TABLE 18
Logistic regression odds ratio results for correlating ACR20 response
status with baseline IL-19 level, adjusted for baseline DAS28

| | | Odds | | | |
|---|---|---|---|---|---|
| | | Ratio or | | | |
| | | Interaction | | Confidence Interval | |
| Group | Comparison | Term | p-value | lower | upper |
| All | 6 mg QD vs | 2.44 | 0.013 | 1.207 | 4.94 |
| | placebo | | | | |
| All | 12 mg QD | 3.62 | <0.0001 | 1.763 | 7.43 |
| | vs placebo | | | | |
| IL-19-high | 6 mg QD vs | 4.66 | 0.004 | 1.613 | 13.48 |
| | placebo | | | | |
| IL-19-high | 12 mg QD | 11.47 | <0.0001 | 3.450 | 38.17 |
| | vs placebo | | | | |
| IL-19-low | 6 mg QD vs | 1.40 | 0.500 | 0.524 | 3.75 |
| | placebo | | | | |
| IL-19-low | 12 mg QD | 1.56 | 0.361 | 0.602 | 4.02 |
| | vs placebo | | | | |
| Interaction | 6 mg QD vs | 3.32 | 0.103 | 0.784 | 14.09 |
| term: | placebo: | | | | |
| treatment | IL-19-high | | | | |
| arm*IL-19 | versus IL- | | | | |
| group | 19-low | | | | |
| Interaction | 12 mg QD | 7.37 | 0.011 | 1.591 | 34.16 |
| term: | vs placebo: | | | | |
| treatment | IL-19-high | | | | |
| arm*IL-19 | versus IL- | | | | |
| group | 19-low | | | | |

FIG. 34, FIG. 35, and Table 18 present the results of the second model for ACR20 response status; the table provides the odds ratio results for ACR20 response status at week 16, adjusted for baseline DAS28. The first two rows of Table 18 are the same as the first two rows of Table 10. As discussed above, these rows provide results for all patients (IL-19-high and IL-19-low); the first two rows in Table 18 provide the odds ratio and p-value for achieving ACR20 response, comparing 6 mg or 12 mg versus placebo, for all patients (IL-19-high and IL-19-low) and when adjusting for baseline DAS28. The results show that the odds of achieving ACR20 was significantly higher in the deucravacitinib treatment arms than in the placebo arm, even after adjusting for baseline DAS28.

The results provided in the third through sixth rows of Table 18 were obtained using the following: ACR20 (Y/N)=treatment arm*IL-19 group+TNF-inhibitor use+baseline weight (continuous)+baseline DAS28 (continuous). The third and fourth rows provide odds ratio results for the IL-19-high group; for the IL-19-high patients, after adjusting for baseline DAS28, the odds of achieving ACR20 was significantly greater than the odds of achieving ACR20 in the placebo arm. In contrast, such significant odds ratio results were not observed in the IL-19-low group (see fifth and sixth rows in Table 18). These results indicate that a high baseline IL-19 level was associated with significant clinical benefit of TYK2 inhibitor treatment (as indicated by significantly greater odds of achieving ACR20 with deucravacitinib treatment compared to the odds of achieving ACR20 with placebo), independent of baseline DAS28.

The results provided in the seventh and eighth rows of Table 18 were obtained using the same model that was used for the third through sixth rows. These results from the interaction term were significant for the 12 mg dose, and for both doses the results show that the ratio of (a) the odds ratio for responding to deucravacitinib versus placebo in the IL-19-high group to (b) the odds ratio for responding to deucravacitinib versus placebo in the IL-19-low group was numerically higher than the null hypothesis (i.e., 1). These results show that compared with a low IL-19 level, a high IL-19 level was associated with a greater odds ratio for responding (as indicated by achieving ACR20) to the TYK2 inhibitor treatment compared to placebo, even after taking baseline DAS28 into account.

FIG. 32, FIG. 33, and Table 19 present the odds ratio results for PASI75 status, adjusted for baseline PASI score. Because there were no PASI75 responders in the IL-19-high placebo group, odds ratios for the IL-19-high group in each treatment arm could not be calculated, and in turn the interaction terms for evaluating the differences of the odds ratios between IL-19-high and IL-19-low groups could not be calculated.

TABLE 19
Logistic regression odds ratio results for correlating PASI75 response
status with baseline IL-19 level, adjusted for baseline PASI score

| | | Odds | | | |
|---|---|---|---|---|---|
| | | Ratio | | Confidence Interval | |
| Group | Comparison | (OR) | p-value | lower | upper |
| All | 6 mg QD vs | 3.052 | 0.011 | 1.293 | 7.20 |
| | placebo | | | | |
| All | 12 mg QD | 6.494 | <0.0001 | 2.665 | 15.82 |
| | vs placebo | | | | |
| IL-19 low | 6 mg QD vs | 0.476 | 0.246 | 0.136 | 1.67 |
| | placebo | | | | |
| IL-19 low | 12 mg QD | 0.896 | 0.852 | 0.280 | 2.86 |
| | vs placebo | | | | |

The results presented in Table 19 were obtained using the following: PASI75 (Y/N)=treatment arm+IL-19 group+treatment arm*IL-19 group+TNF-inhibitor use+baseline weight (continuous)+baseline PASI score. The results in the first and second rows show that the patients in both treatment arms had a significant treatment benefit, as indicated by a significantly higher odds of achieving PASI75 with deucravacitinib treatment compared to the odds of achieving PASI75 with placebo treatment, even after adjusting for baseline PASI score. The results in the third and fourth rows show that the IL-19-low group did not show a significant treatment benefit. These results are consistent with the conclusion that a high baseline IL-19 level was associated with greater treatment benefit.

Table 20 provides the logistic regression model results, when patients were categorized into four groups, based on baseline IL-19 serum level and baseline PASI score, each dichotomized according to median value (as described above for BD2): IL-19-high-PASI-high; IL-19-high-PASI-low; IL-19-low-PASI-high; and IL-19-low-PASI-low. Results for the IL-19-high-PASI-high group and for the IL-19-high-PASI-low group could not be calculated because there were no PASI75 responders in the IL-19-high placebo group. The results in Table 20 show that IL-19-low patients did not benefit from deucravacitinib treatment compared with placebo, regardless of baseline PASI status.

TABLE 20
Logistic regression odds ratio results for correlating PASI75 response
status with baseline IL-19 level and baseline PASI status

| | | Odds | | | |
|---|---|---|---|---|---|
| | | Ratio | | Confidence Interval | |
| Group | Comparison | (OR) | p-value | lower | upper |
| All | 6 mg QD vs | 2.880 | 0.014 | 1.240 | 6.69 |
| | placebo | | | | |
| All | 12 mg QD | 5.781 | <0.0001 | 2.436 | 13.72 |
| | vs placebo | | | | |
| IL-19-low- | 6 mg QD vs | 1.244 | 0.848 | 0.133 | 11.63 |
| PASI high | placebo | | | | |
| IL-19-low- | 12 mg QD | 2.845 | 0.319 | 0.364 | 22.25 |
| PASI high | vs placebo | | | | |
| IL-19-low- | 6 mg QD vs | 0.321 | 0.169 | 0.063 | 1.62 |
| PASI low | placebo | | | | |
| IL-19-low- | 12 mg QD | 0.380 | 0.246 | 0.074 | 1.95 |
| PASI low | vs placebo | | | | |

### IL-17A

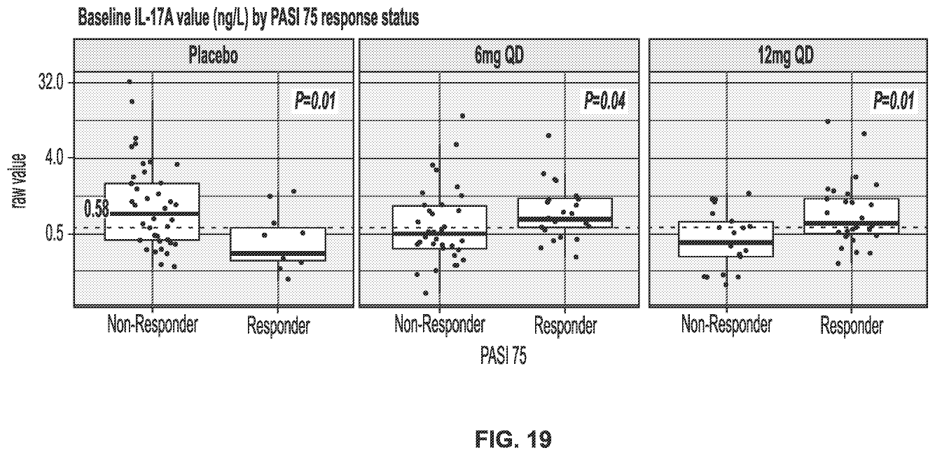

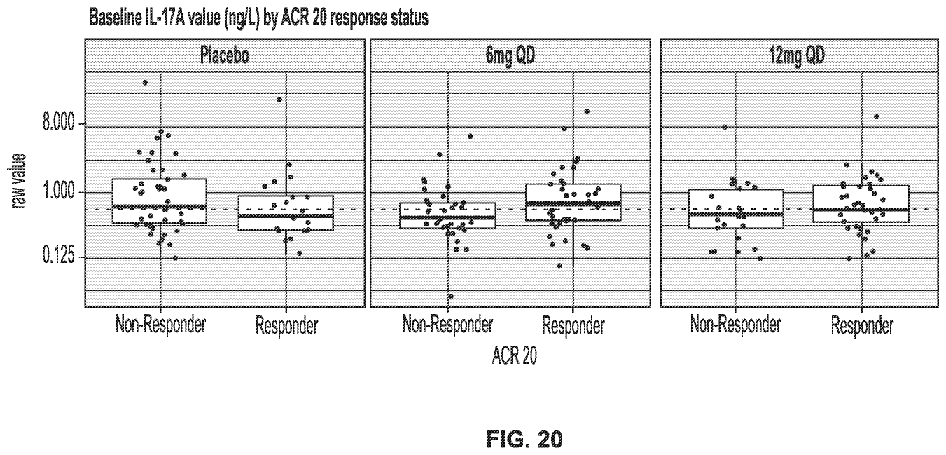

The same analyses were performed for baseline IL-17A level. The median baseline level of IL-17A (for all 202 subjects having baseline IL-17A data) was 0.575 ng/L. FIG. 19 and FIG. 20 provide box plots of non-responders and responders (according to PASI75 response status and ACR20 response status, respectively) at week 16 in each treatment arm, with the dashed horizontal line corresponding to the median baseline IL-17A level, and the solid horizontal line in each box representing the median baseline IL-17A level for that box plot. Table 21 provides p-values for comparing baseline mean or baseline median IL-17A level in responders versus non-responders, for each treatment arm; for the t-test to compare mean values, IL-17A levels were log 2 transformed prior to calculating the means for responders and non-responders.

TABLE 21
p-values for each treatment arm, comparing baseline
IL-17A level in responders versus non-responders

| | t-test comparing mean | Wilcoxon rank sum test |
|---|---|---|
| | baseline IL-17A level* in | comparing median baseline |
| | responders versus non- | IL-17A level in responders |
| Treatment arm | responders | versus non-responders |
| | PASI75 responders versus non-responders | |
| Placebo | 0.00392 | 0.01025 |
| 6 mg QD | 0.13572 | 0.04445 |
| 12 mg QD | 0.00424 | 0.01246 |
| | ACR20 responders versus non-responders | |
| Placebo | 0.27467 | 0.22038 |
| 6 mg QD | 0.10836 | 0.09355 |
| 12 mg QD | 0.32899 | 0.26379 |

*log2 transformed

Higher baseline IL-17A levels were observed in PASI75 non-responders versus responders for placebo, while higher baseline IL-17A levels were observed in responders compared to non-responders for the deucravacitinib treatment groups. See FIG. 19. A comparatively higher level of IL-17A (like BD2 and IL-19) can be used to identify patients who have better skin responses to deucravacitinib than the responses in patients with a comparatively lower level of IL-17A. Similar results were observed for ACR20 (see FIG. 20).

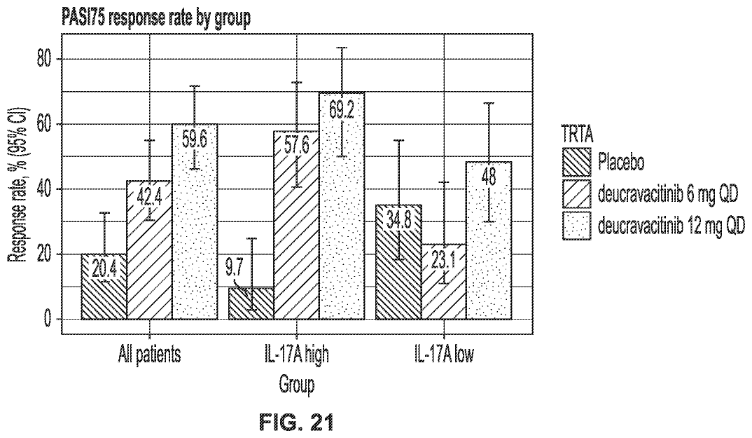

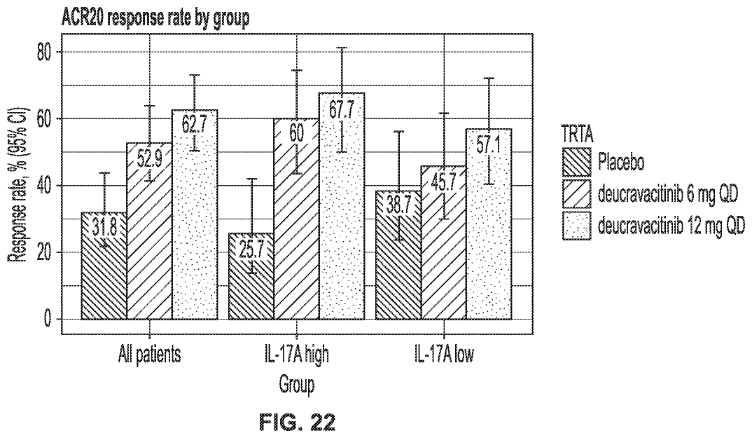

FIG. 21 and FIG. 22 are barplots showing the response rate in each treatment arm, for achieving PASI75 or ACR20 response status at week 16, for patients in IL-17A-high and IL-17A-low groups (dichotomized according to median IL-17A baseline level). For PsA patients with IL-17A levels above median (the “IL-17A high” group), the PASI75 response rate was higher in both 6 mg QD and 12 mg QD treatment arms compared to placebo, while there was no significant PASI75 response difference in PsA patients with baseline IL-17A levels at or below median (the “IL-17A low” group). Similarly, the ACR20 response rate was higher in both 6 mg QD and 12 mg QD treatment arms compared to placebo in the IL-17-high group, whereas the ACR20 response rate did not appear to differ among the treatment arms for patients in the IL-17-low group. Table 22 below provides the number of responders and non-responders, in IL-17A-low and IL-17A-high patient groups (classified according to median IL-17A serum levels measured at baseline in PsA patients), by treatment arm.

TABLE 22
Number of non-responders and responders in IL-17A-
low and IL-17A-high groups, by treatment arm

| | | | |
|---|---|---|---|
| | Response | Below/above | |
| | status at | median baseline | Number of |
| Treatment arm | week 16 | IL-17A level | subjects |

PASI75 response status

| | | | |
|---|---|---|---|
| Placebo | Non-responder | Below | 15 |
| | | Above | 28 |
| | Responder | Below | 8 |
| | | Above | 3 |
| 6 mg QD | Non-responder | Below | 20 |
| | | Above | 14 |
| | Responder | Below | 6 |
| | | Above | 19 |
| 12 mg QD | Non-responder | Below | 13 |
| | | Above | 8 |
| | Responder | Below | 12 |
| | | Above | 18 |

ACR20 response status

| | | | |
|---|---|---|---|
| Placebo | Non-responder | Below | 19 |
| | | Above | 26 |
| | Responder | Below | 12 |
| | | Above | 9 |
| 6 mg QD | Non-responder | Below | 19 |
| | | Above | 14 |
| | Responder | Below | 16 |
| | | Above | 21 |
| 12 mg QD | Non-responder | Below | 15 |
| | | Above | 10 |
| | Responder | Below | 20 |
| | | Above | 21 |

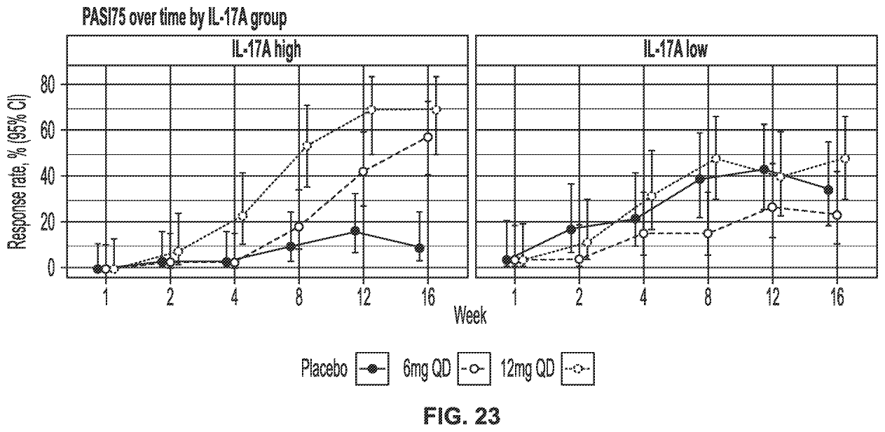

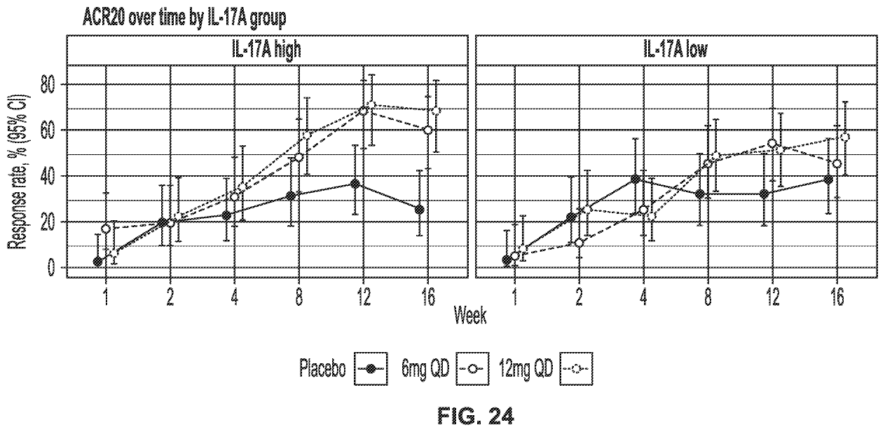

FIG. 23 and FIG. 24 provide line plots of response rate (PASI75 and ACR20, respectively) over time, for IL-17A baseline high and IL-17A baseline low groups. A dose-dependent PASI75 response over time was observed in IL-17A-high but not in IL-17A-low PsA patients. In addition, higher ACR20 response rates over time were observed in the deucravacitinib treatment arms compared to placebo in IL-17A-high but not in IL-17A-low groups.

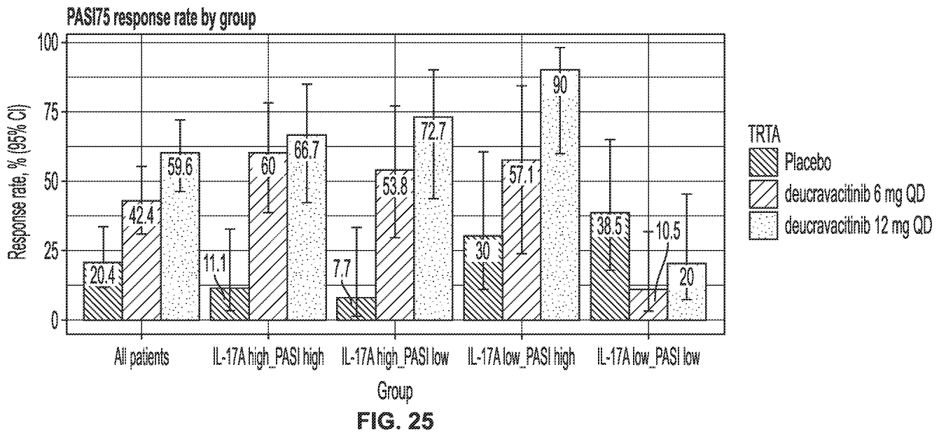

PsA patients were also categorized by their baseline IL-17A expression level (above or below median) and baseline PASI status (above or below median) into four groups: IL-17A-high and PASI-high; IL-17A-high and PASI-low; IL-17A-low and PASI-high; and IL-17A-low and PASI-low. Table 23 provides the number of patients within each of the aforementioned groups. FIG. 25 is a barplot of PASI75 response status at week 16 in each treatment arm, for all subjects as categorized into the aforementioned four groups. The barplot shows that baseline IL-17A level and baseline PASI are associated with PASI75 response to deucravacitinib, and that baseline IL-17A level is associated with PASI75 response to deucravacitinib even for patients in the low-PASI group (see “IL-17A-high PASI-low” group in FIG. 25).

TABLE 23
Number of PASI75 non-responders and responders in IL-17A-
high-PASI-high, IL-17A-high-PASI-low, IL-17A-low-PASI-
high, and IL-17A-low-PASI-low groups, by treatment arm

| Treatment | PASI75 | | Number of |
|---|---|---|---|
| arm | response status | Group | subjects |
| Placebo | Responder | IL-17A-high-PASI-high | 2 |
| | | IL-17A-high-PASI-low | 1 |
| | | IL-17A-low-PASI-high | 3 |
| | | IL-17A-low-PASI-low | 5 |
| | Non-responder | IL-17A-high-PASI-high | 16 |
| | | IL-17A-high-PASI-low | 12 |
| | | IL-17A-low-PASI-high | 7 |
| | | IL-17A-low-PASI-low | 8 |
| 6 mg QD | Responder | IL-17A-high-PASI-high | 12 |
| | | IL-17A-high-PASI-low | 7 |
| | | IL-17A-low-PASI-high | 4 |
| | | IL-17A-low-PASI-low | 2 |
| | Non-responder | IL-17A-high-PASI-high | 8 |
| | | IL-17A-high-PASI-low | 6 |
| | | IL-17A-low-PASI-high | 3 |
| | | IL-17A-low-PASI-low | 17 |
| 12 mg QD | Responder | IL-17A-high-PASI-high | 10 |
| | | IL-17A-high-PASI-low | 8 |
| | | IL-17A-low-PASI-high | 9 |
| | | IL-17A-low-PASI-low | 3 |
| | Non-responder | IL-17A-high-PASI-high | 5 |
| | | IL-17A-high-PASI-low | 3 |
| | | IL-17A-low-PASI-high | 1 |
| | | IL-17A-low-PASI-low | 12 |

Logistic regression models were fit as described above for BD2 and IL-19. For response measures ACR20 and PASI75, the odds ratio for each of the dosed arms versus placebo, in IL-17A-high and IL-17A-low groups, was generated. The model for ACR20 was: ACR20 (Y/N)=treatment arm+IL-17A group+treatment arm*IL-17A group+TNF-inhibitor use+baseline weight (continuous). The same model was fit for PASI75 response status: PASI75 (Y/N)=treatment arm+IL-17A group+treatment arm*IL-17A group+TNF-inhibitor use+baseline weight (continuous). The p-value of the interaction term was used to identify whether the OR for achieving the response was significantly different between IL-17A-high and IL-17A-low groups. FIGS. 26-31 are forest plots summarizing the logistic regression results of these models (for IL-17A and other biomarkers described herein). The statistical model used to analyze the effect of baseline IL-17A level was run with each treatment arm (6 mg QD, 12 mg QD, and placebo) as a separate predictor (see FIG. 26, FIG. 27, and Table 24A for ACR20 results; see FIG. 29, FIG. 30 and Table 25A for PASI75 results) and with the deucravacitinib treatment arms combined (see FIG. 28 and Table 24B for ACR20 results; see FIG. 31 and Table 25B for PASI75 results).

TABLE 24A
Logistic regression odds ratio results for correlating
ACR20 response status with baseline IL-17A level

| | | Odds | | | |
|---|---|---|---|---|---|
| | | Ratio or | | | |
| | | Interaction | | Confidence Interval | |
| Group | Comparison | Term | p-value | lower | upper |
| All | 6 mg QD vs | 2.46 | 0.012 | 1.214 | 4.96 |
| | placebo | | | | |
| All | 12 mg QD | 3.61 | <0.0001 | 1.759 | 7.41 |
| | vs placebo | | | | |
| IL-17A | 6 mg QD vs | 4.30 | 0.005 | 1.555 | 11.90 |
| high | placebo | | | | |
| IL-17A | 12 mg QD | 5.95 | 0.001 | 2.031 | 17.43 |
| high | vs placebo | | | | |
| IL-17A low | 6 mg QD vs | 1.39 | 0.518 | 0.514 | 3.74 |
| | placebo | | | | |
| IL-17A low | 12 mg QD | 2.14 | 0.131 | 0.797 | 5.74 |
| | vs placebo | | | | |
| Interaction | 6 mg QD vs | 3.10 | 0.118 | 0.749 | 12.84 |
| term: | placebo: | | | | |
| treatment | IL-17A high | | | | |
| arm*IL- | versus IL- | | | | |
| 17A group | 17A low | | | | |
| Interaction | 12 mg QD | 2.78 | 0.170 | 0.645 | 11.99 |
| term: | vs placebo: | | | | |
| treatment | IL-17A high | | | | |
| arm*IL- | versus IL- | | | | |
| 17A group | 17A low | | | | |

TABLE 24B
Logistic regression odds ratio results for correlating
ACR20 response status with baseline IL-17A level
(deucravacitinib treatment arms combined)

| | | Odds | | | |
|---|---|---|---|---|---|
| | | Ratio or | | | |
| | | Interaction | | Confidence Interval | |
| Group | Comparison | Term | p-value | lower | upper |
| All | Deucravacitinib | 2.961 | 0.0006 | 1.588 | 5.519 |
| | vs placebo | | | | |
| IL-17A | Deucravacitinib | 4.990 | 0.0006 | 2.003 | 12.432 |
| high | vs placebo | | | | |
| IL-17A | Deucravacitinib | 1.723 | 0.2157 | 0.727 | 4.115 |
| low | vs placebo | | | | |
| Interaction | Deucravacitinib | 2.886 | 0.0990 | 0.819 | 10.165 |
| term: | vs placebo: IL- | | | | |
| treatment | 17A high | | | | |
| arm | versus IL-17A | | | | |
| IL-17A | low | | | | |
| group | | | | | |

TABLE 25A
Logistic regression odds ratio results for correlating
PASI75 response status with baseline IL-17A level

| | | Odds Ratio or | | Confidence | |
|---|---|---|---|---|---|
| | | Interaction | | Interval | |
| Group | Comparison | Term | p-value | lower | upper |
| All | 6 mg QD vs | 2.880 | 0.014 | 1.240 | 6.69 |
| | placebo | | | | |
| All | 12 mg QD | 5.781 | <0.0001 | 2.436 | 13.72 |
| | vs placebo | | | | |
| IL-17A | 6 mg QD vs | 12.643 | <0.0001 | 3.191 | 50.10 |
| high | placebo | | | | |
| IL-17A | 12 mg QD | 20.916 | <0.0001 | 4.868 | 89.87 |
| high | vs placebo | | | | |
| IL-17A low | 6 mg QD vs | 0.553 | 0.358 | 0.157 | 1.95 |
| | placebo | | | | |
| IL-17A low | 12 mg QD | 1.730 | 0.361 | 0.534 | 5.60 |
| | vs placebo | | | | |
| Interaction | 6 mg QD vs | 22.851 | 0.001 | 3.529 | 147.95 |
| term: | placebo: | | | | |
| treatment | IL-17A high | | | | |
| arm*IL- | versus IL- | | | | |
| 17A group | 17A low | | | | |
| Interaction | 12 mg QD | 12.089 | 0.009 | 1.839 | 79.45 |
| term: | vs placebo: | | | | |
| treatment | IL-17A high | | | | |
| arm*IL- | versus IL- | | | | |
| 17A group | 17A low | | | | |

TABLE 25B
Logistic regression odds ratio results for correlating
PASI75 response status with baseline IL-17A level
(deucravacitinib treatment arms combined)

| | | Odds Ratio or | | Confidence | |
|---|---|---|---|---|---|
| | | Interaction | | Interval | |
| Group | Comparison | Term | p-value | lower | upper |
| All | Deucravacitinib | 3.998 | 0.0004 | 1.867 | 8.563 |
| | vs placebo | | | | |
| IL-17A | Deucravacitinib | 15.763 | <0.0001 | 4.273 | 58.148 |
| high | vs placebo | | | | |
| IL-17A | Deucravacitinib | 1.014 | 0.9787 | 0.358 | 2.872 |
| low | vs placebo | | | | |
| Interaction | Deucravacitinib | 15.541 | 0.0013 | 2.914 | 82.867 |
| term: | vs placebo: IL- | | | | |
| treatment | 17A high | | | | |
| arm * | versus IL-17A | | | | |
| IL-17A | low | | | | |
| group | | | | | |

High baseline IL-17A level was associated with a significant treatment benefit, as indicated by significantly higher odds of achieving ACR20 and PASI75, respectively, in response to deucravacitinib compared to placebo (see FIG. 28 and second row of Table 24B for ACR20 results; see FIG. 31 and Table 25B for PASI75 results). This result was observed at each treatment dose (see FIG. 26, FIG. 27, and third and fourth rows of Table 24A for ACR20; see FIG. 29, FIG. 30, and third and fourth rows of Table 25A for PASI75). Such a result was not observed for the IL-17A-low group (see the third rows of Table 24B and Table 25B, as well as the fifth and sixth rows of Table 24A and Table 25A). Furthermore, compared with the IL-17A-low group, the IL-17A high group had a numerically greater ACR20 treatment benefit (see seventh and eighth rows of Table 24A and last row of Table 24B) and a statistically significantly greater PASI75 treatment benefit (see seventh and eighth rows of Table 25A and the last row of Table 25B).

Additional logistic regression models were fit for baseline IL-17A serum level and response status, as described above for BD2 and IL-19. Table 26 and Table 27 provide the results of the second model, which accounts for baseline disease activity, as described further below.

TABLE 26
Logistic regression odds ratio results for correlating ACR20 response
status with baseline IL-17A level, adjusted for baseline DAS28

| | | Odds Ratio or | | Confidence | |
|---|---|---|---|---|---|
| | | Interaction | | Interval | |
| Group | Comparison | Term | p-value | lower | upper |
| All | 6 mg QD vs | 2.44 | 0.013 | 1.207 | 4.94 |
| | placebo | | | | |
| All | 12 mg QD | 3.62 | <0.0001 | 1.763 | 7.43 |
| | vs placebo | | | | |
| IL-17A- | 6 mg QD vs | 4.27 | 0.005 | 1.542 | 11.83 |
| high | placebo | | | | |
| IL-17A- | 12 mg QD | 5.92 | 0.001 | 2.022 | 17.36 |
| high | vs placebo | | | | |
| IL-17A-low | 6 mg QD vs | 1.39 | 0.519 | 0.514 | 3.74 |
| | placebo | | | | |
| IL-17A-low | 12 mg QD | 2.16 | 0.128 | 0.802 | 5.80 |
| | vs placebo | | | | |
| Interaction | 6 mg QD vs | 3.08 | 0.121 | 0.743 | 12.77 |
| term: | placebo: | | | | |
| treatment | IL-17A-high | | | | |
| arm*IL- | versus IL- | | | | |
| 17A group | 17A-low | | | | |
| Interaction | 12 mg QD | 2.75 | 0.176 | 0.635 | 11.87 |
| term: | vs placebo: | | | | |
| treatment | IL-17A-high | | | | |
| arm*IL- | versus IL- | | | | |
| 17A group | 17A-low | | | | |

Table 26 presents the results of the second model for ACR20 response status; the table provides the odds ratio results for ACR20 response status at week 16, adjusting for baseline DAS28. See also FIG. 34 and FIG. 35. The first two rows in Table 26 show that for all patients (IL-17A-high and IL-17A-low) the odds of achieving an ACR20 response was significantly higher in the deucravacitinib treatment arms than in the placebo arm, even after adjusting for baseline DAS28.

The results provided in the third through sixth rows of Table 26 were obtained using the following: ACR20 (Y/N)=treatment arm*IL-17A group+TNF-inhibitor use+baseline weight (continuous)+baseline DAS28 (continuous). The third and fourth rows provide odds ratio results for the IL-17A-high group; these results indicate that for the IL-17A-high patients, after adjusting for baseline DAS28, the odds of achieving ACR20 in each deucravacitinib treatment arm was significantly greater than the odds of achieving ACR20 response in the placebo arm. In contrast, such significant odds ratio results were not observed in the IL-17A-low groups (see the fifth and sixth rows in Table 26). These results indicate that a high baseline IL-17A level was associated with significant treatment benefit (as indicated by significantly higher odds of achieving ACR20 with deucravacitinib treatment, compared to the odds of achieving ACR20 in the placebo arm), independent of baseline DAS28.

The results provided in the seventh and eighth rows in Table 26 were obtained using the same model that was used for the third through sixth rows. These results from the interaction terms were not statistically significant but are consistent with other results and indicate that the ratio of (a) the odds ratio of responding to deucravacitinib versus placebo in the IL-17A-high group to (b) the odds ratio for responding to deucravacitinib versus placebo in the IL-17A-low group was numerically higher than the null hypothesis (i.e., 1).

TABLE 27
Logistic regression odds ratio results for correlating
PASI75 response status with baseline IL-17A level,
adjusted for baseline PASI score

| | | Odds Ratio or | | Confidence | |
|---|---|---|---|---|---|
| | | Interaction | | Interval | |
| Group | Comparison | Term | p-value | lower | upper |
| All | 6 mg QD vs | 3.052 | 0.011 | 1.293 | 7.20 |
| | placebo | | | | |
| All | 12 mg QD | 6.494 | <0.0001 | 2.665 | 15.82 |
| | vs placebo | | | | |
| IL-17A | 6 mg QD vs | 13.452 | <0.0001 | 3.317 | 54.55 |
| high | placebo | | | | |
| IL-17A | 12 mg QD | 23.963 | <0.0001 | 5.365 | 107.03 |
| high | vs placebo | | | | |
| IL-17A low | 6 mg QD vs | 0.588 | 0.412 | 0.165 | 2.09 |
| | placebo | | | | |
| IL-17A low | 12 mg QD | 1.812 | 0.326 | 0.554 | 5.93 |
| | vs placebo | | | | |
| Interaction | 6 mg QD vs | 22.889 | 0.001 | 3.480 | 150.57 |
| term: | placebo: | | | | |
| treatment | IL-17A high | | | | |
| arm*IL- | versus IL- | | | | |
| 17A group | 17A low | | | | |
| Interaction | 12 mg QD | 13.222 | 0.008 | 1.952 | 89.58 |
| term: | vs placebo: | | | | |
| treatment | IL-17A high | | | | |
| arm*IL- | versus IL- | | | | |
| 17A group | 17A low | | | | |

Table 27 presents the odds ratio results for PASI75 status, adjusting for baseline PASI score. The first two rows in Table 27 provide the odds ratio results for PASI75 status at week 16, when adjusting for baseline PASI score, for all patients (IL-17A-high and IL-17A-low), and thus these results are the same as the results provided in the first two rows in Table 11 and Table 19.

Results for the third through sixth rows of Table 27 are based on the following: PASI75 (Y/N)=treatment arm*IL-17A group+TNF-inhibitor use+baseline weight (continuous)+baseline PASI score (continuous). FIG. 32 and FIG. 33 provide forest plots summarizing the results of the above model. The third and fourth rows provide odds ratio results for the IL-17A-high group; these results indicate that for the IL-17A-high patients, after adjusting for baseline PASI score, the odds of achieving PASI75 response in each deucravacitinib treatment arm was significantly greater than the odds of achieving PASI75 response in the placebo arm. In contrast, such significant odds ratio results were not observed for IL-17A-low patients (see fifth and sixth rows in Table 27). These results indicate that a high baseline IL-17A level was associated with significant clinical benefit (as indicated by achieving PASI75) of TYK2 inhibitor treatment compared to placebo, independent of baseline PASI score.

The results provided in the seventh and eighth rows in Table 27 were obtained using the same model that was used for the third through sixth rows. The ratio of (a) the odds ratio for achieving PASI75 after treatment with deucravacitinib versus placebo in the IL-17A-high group to (b) the odds ratio for achieving PASI75 after treatment with deucravacitinib versus placebo in the IL-17A-low group was statistically significantly higher than the null hypothesis (i.e., 1). These results show that compared with a low IL-17A level, a high IL-17A level was associated with a significantly greater odds ratio for responding (as indicated by achieving PASI75) to TYK2 inhibitor treatment compared to placebo, even after adjusting for baseline PASI.

Another logistic regression model was fit for PASI75 response status, to assess the individual predictive value of baseline IL-17A level and baseline PASI score, each dichotomized as described above. This model generated the odds ratio of each of the dosed arms versus placebo, for each of the four baseline IL-17A-PASI groups (i.e., IL-17A-high-PASI-high; IL-17A-high-PASI-low; IL-17A-low-PASI-high; IL-17A-low-PASI-low; see FIG. 25). This model was: PASI75 (Y/N)=treatment arm+baseline IL-17A-PASI group+treatment arm*baseline IL-17A-PASI group+TNF-inhibitor use+baseline weight (continuous). Table 28 presents the results of this model.

TABLE 28
Logistic regression odds ratio results for correlating PASI75
response status with baseline IL-17A and baseline PASI

| | | Odds | | Confidence Interval | |
|---|---|---|---|---|---|
| Group | Comparison | Ratio | p-value | lower | upper |
| All | 6 mg QD vs | 2.880 | 0.014 | 1.240 | 6.689 |
| | placebo | | | | |
| Al | 12 mg QD vs | 5.781 | <0.0001 | 2.436 | 13.720 |
| | placebo | | | | |
| IL-17A | 6 mg QD vs | 12.355 | 0.004 | 2.186 | 69.830 |
| high-PASI | placebo | | | | |
| high | | | | | |
| IL-17A | 12 mg QD vs | 15.937 | 0.003 | 2.573 | 98.727 |
| high-PASI | placebo | | | | |
| high | | | | | |
| IL-17A | 6 mg QD vs | 13.247 | 0.030 | 1.290 | 136.080 |
| high-PASI | placebo | | | | |
| low | | | | | |
| IL-17A | 12 mg QD vs | 30.955 | 0.006 | 2.696 | 355.407 |
| high-PASI | placebo | | | | |
| low | | | | | |
| IL-17A | 6 mg QD vs | 3.005 | 0.294 | 0.384 | 23.481 |
| low-PASI | placebo | | | | |
| high | | | | | |
| IL-17A | 12 mg QD vs | 20.919 | 0.017 | 1.732 | 252.609 |
| low-PASI | placebo | | | | |
| high | | | | | |
| IL-17A | 6 mg QD vs | 0.188 | 0.075 | 0.030 | 1.185 |
| low-PASI | placebo | | | | |
| low | | | | | |
| IL-17A | 12 mg QD vs | 0.415 | 0.310 | 0.076 | 2.268 |
| low-PASI | placebo | | | | |
| low | | | | | |

In Table 28, the first two rows provide the odds ratios comparing the odds of achieving PASI75 for each deucravacitinib treatment arm versus placebo, using the following: PASI75 (Y/N)=treatment arm+TNF-inhibitor use+baseline weight (continuous). The odds ratios of the third through tenth rows provide results from the following: PASI75 (Y/N)=treatment arm+baseline IL-17A-PASI group+treatment arm*baseline IL-17A-PASI group+TNF-inhibitor use+baseline weight (continuous). In both treatment arms, patients with a higher baseline IL-17A level, regardless of baseline PASI score status, had significantly greater odds of responding (as indicated by PASI75) to deucravacitinib compared to the odds of responding to placebo (see third, fourth, fifth, and sixth rows). In contrast, most patient groups with a lower baseline IL-17A level did not (see seventh, ninth, and tenth rows). Among the IL-17A-low patient groups, only the IL-17A-low-PASI high group showed a statistically significantly higher odds of responding to deucravacitinib compared to the odds of responding to placebo (see eighth row).

### C-Reactive Protein (CRP)

Analyses like those presented for other proteins were performed for baseline CRP levels measured in PsA patients. The median baseline level of CRP (for all 203 subjects having CRP baseline data) was 8.31 mg/L.

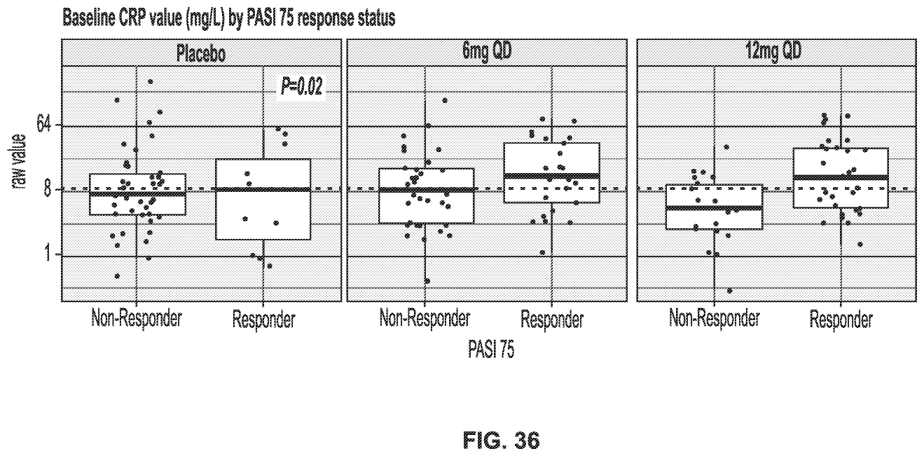

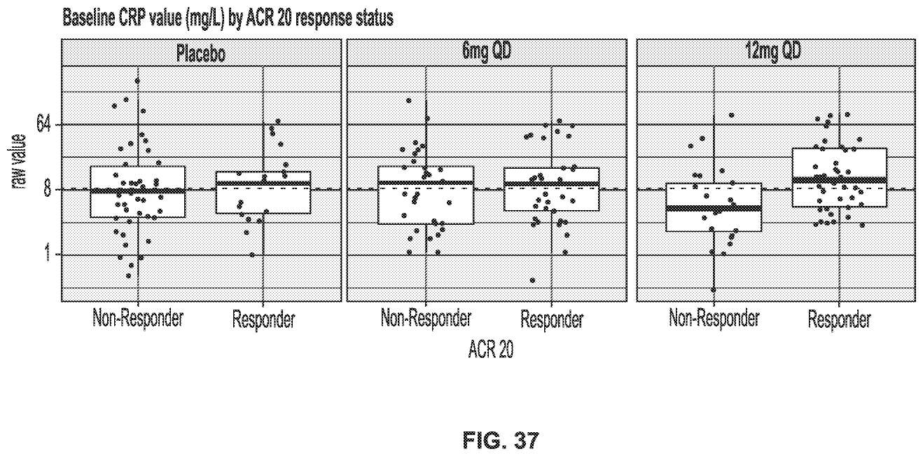

FIG. 36 and FIG. 37 provide box plots of non-responders and responders (according to PASI75 response status and ACR20 response status, respectively) at week 16 in each treatment arm, with the dashed horizontal line corresponding to the median baseline CRP level, and the solid horizontal line in each box representing the median baseline CRP level for that box plot.

Table 29 provides p-values for comparing baseline mean or baseline median CRP level in responders versus non-responders, for each treatment arm; for the t-test to compare mean values, CRP level was log 2 transformed prior to calculating the means for responders and non-responders. For each of PASI75 and ACR20, the baseline CRP levels were significantly higher in the responders only for the 12 mg QD treatment arm, whereas there were no significant differences between responders and non-responders in the placebo and 6 mg QD arms.

TABLE 29
p-values for each treatment arm, comparing baseline
CRP level in responders versus non-responders

| | t-test comparing mean | Wilcoxon rank sum test |
|---|---|---|
| | baseline CRP level* in | comparing median baseline |
| | responders versus non- | CRP level in responders |
| Treatment arm | responders | versus non-responders |
| | PASI75 responders versus non-responders | |
| Placebo | 0.65697 | 0.73112 |
| 6 mg QD | 0.22193 | 0.16506 |
| 12 mg QD | 0.00196 | 0.00578 |
| | ACR20 responders versus non-responders | |
| Placebo | 0.74752 | 0.61049 |
| 6 mg QD | 0.66507 | 0.67188 |
| 12 mg QD | 0.0073 | 0.00899 |

*log2 transformed

Patients were dichotomized into a CRP-low group (patients having a baseline CRP level at or below median) and a CRP-high group (patients having a baseline CRP level above median). Table 30 provides the number of responders and non-responders in CRP-low and CRP-high groups, by treatment arm.

TABLE 30
Number of non-responders and responders in CRP-
low and CRP-high groups, by treatment arm

| | | | |
|---|---|---|---|
| | Response | Below/above | |
| Treatment | status at | median baseline | Number of |
| arm | week 16 | CRP level | subjects |

PASI75 response status

| | | | |
|---|---|---|---|
| Placebo | Non-responder | Below | 24 |
| | | Above | 19 |
| | Responder | Below | 6 |
| | | Above | 5 |
| 6 mg QD | Non-responder | Below | 17 |
| | | Above | 17 |
| | Responder | Below | 9 |
| | | Above | 16 |
| 12 mg QD | Non-responder | Below | 14 |
| | | Above | 7 |
| | Responder | Below | 13 |
| | | Above | 18 |

ACR20 response status

| | | | |
|---|---|---|---|
| Placebo | Non-responder | Below | 24 |
| | | Above | 21 |
| | Responder | Below | 10 |
| | | Above | 11 |
| 6 mg QD | Non-responder | Below | 16 |
| | | Above | 17 |
| | Responder | Below | 18 |
| | | Above | 19 |
| 12 mg QD | Non-responder | Below | 16 |
| | | Above | 9 |
| | Responder | Below | 18 |
| | | Above | 24 |

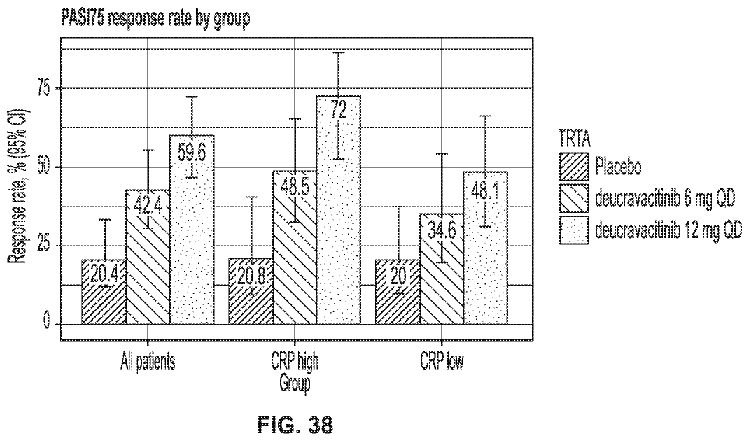

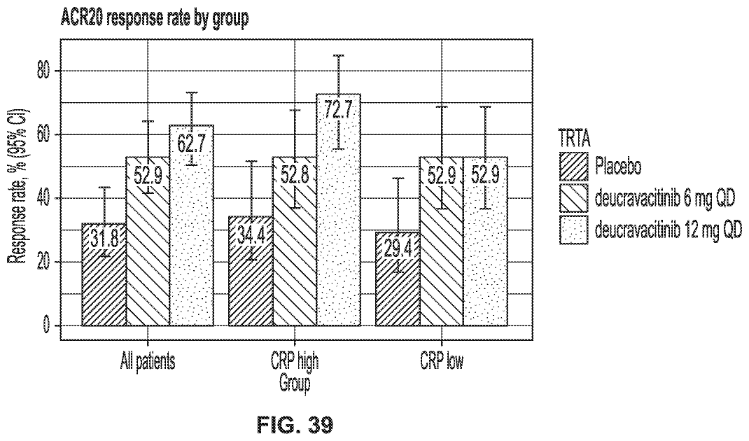

FIG. 38 and FIG. 39 are bargraphs showing the response rate in each treatment arm, for achieving PASI75 or ACR20 response status at week 16, for CRP-high and CRP-low groups. For the both CRP-high and CRP-low groups, the PASI75 response rate showed a dose response effect, such that the response rate was higher in the 6 mg deucravacitinib treatment arm than in the placebo arm, and the response rate was even higher in the 12 mg deucravacitinib treatment arm than in the 6 mg treatment arm. Similar results were observed for the ACR20 response rate, except that there was no difference in response rate between the 6 mg and 12 mg treatment arms in the CRP-low group.

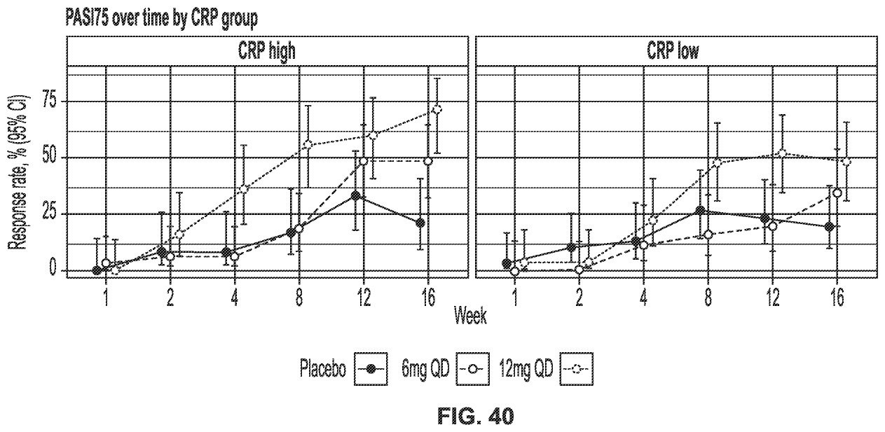

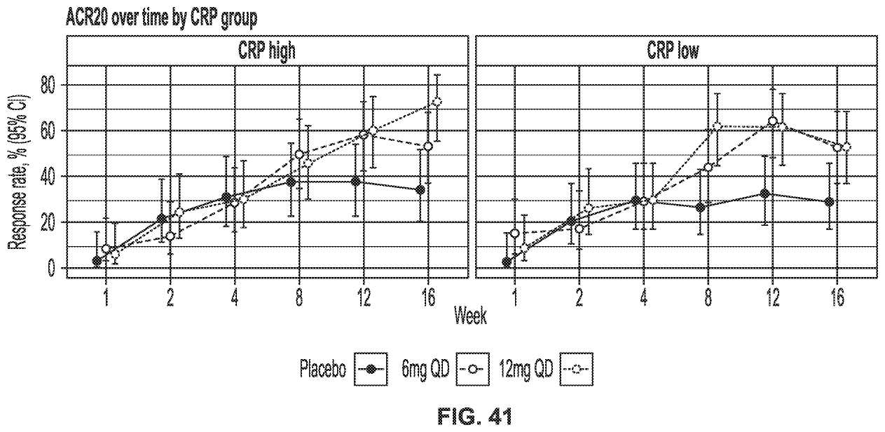

FIG. 40 and FIG. 41 are line plots of response rate (PASI75 and ACR20, respectively) over time, for the CRP-high and CRP-low groups. The deucravacitinib treatment arms showed higher response rates than the placebo arm; however, the CRP-high and the CRP-low groups did not appear to differ in responsiveness.

PsA patients were also dichotomized by their baseline CRP level and baseline PASI status into four groups: CRP-high and PASI-high; CRP-high and PASI-low; CRP-low and PASI-high; and CRP-low and PASI-low. Table 30 provides the number of PsA patients within each of the four aforementioned groups.

TABLE 31
Number of PASI75 non-responders and responders in CRP-
high-PASI-high, CRP-high-PASI-low, CRP-low-PASI-high,
and CRP-low-PASI-low groups, by treatment arm

| Treatment | PASI75 | | Number of |
|---|---|---|---|
| arm | response status | Group | subjects |
| Placebo | Responder | CRP-high-PASI-high | 2 |
| | | CRP-high-PASI-low | 3 |
| | | CRP-low-PASI-high | 3 |
| | | CRP-low-PASI-low | 3 |
| | Non-responder | CRP-high-PASI-high | 13 |
| | | CRP-high-PASI-low | 6 |
| | | CRP-low-PASI-high | 10 |
| | | CRP-low-PASI-low | 14 |
| 6 mg QD | Responder | CRP-high-PASI-high | 11 |
| | | CRP-high-PASI-low | 5 |
| | | CRP-low-PASI-high | 5 |
| | | CRP-low-PASI-low | 4 |
| | Non-responder | CRP-high-PASI-high | 4 |
| | | CRP-high-PASI-low | 13 |
| | | CRP-low-PASI-high | 7 |
| | | CRP-low-PASI-low | 10 |
| 12 mg QD | Responder | CRP-high-PASI-high | 10 |
| | | CRP-high-PASI-low | 8 |
| | | CRP-low-PASI-high | 10 |
| | | CRP-low-PASI-low | 3 |
| | Non-responder | CRP-high-PASI-high | 4 |
| | | CRP-high-PASI-low | 3 |
| | | CRP-low-PASI-high | 2 |
| | | CRP-low-PASI-low | 12 |

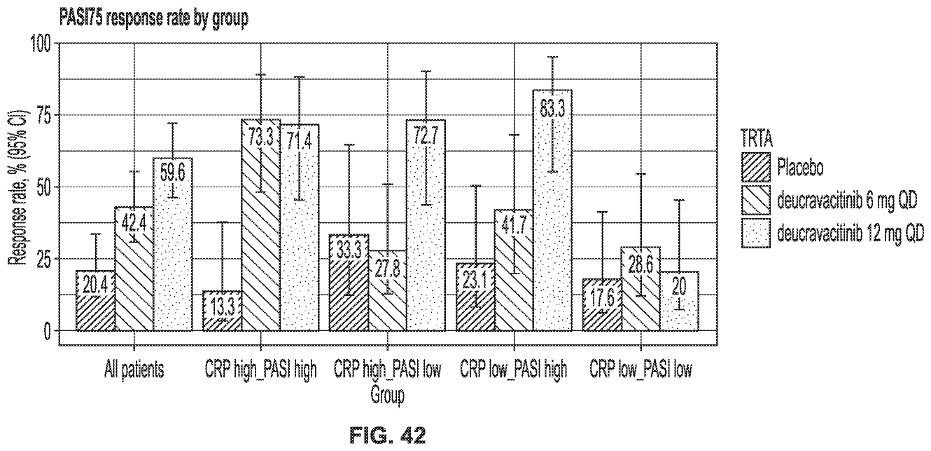

FIG. 42 is a bargraph of PASI75 response status at week 16 in each treatment arm for all subjects, as categorized into the aforementioned four groups. The bargraph shows that baseline CRP level did not exhibit a consistent association with PASI75 response.

Logistic regression models were fit as described above for other proteins. Models were fit for response measures ACR20 and PASI75, to generate the odds ratio of each of the dosed arms versus placebo, in CRP-high and CRP-low groups. For the first logistic regression model, the following were used:
For ACR20 response status: ACR20 (Y/N)=treatment arm+CRP group+treatment arm*CRP group+TNF-inhibitor use+baseline weight (continuous).
For PASI75 response status: PASI75 (Y/N)=treatment arm+CRP group+treatment arm*CRP group+TNF-inhibitor use+baseline weight (continuous).
   The p-value of the interaction term was used to identify whether the odds ratio for achieving the response was significantly different between CRP-high and CRP-low groups. FIGS. 26-31 are forest plots summarizing the logistic regression results of these models (for CRP and the biomarkers described herein). Table 32 and Table 33 provide the odds ratio results for CRP. The statistical model used to analyze the effect of baseline CRP level was run with each treatment arm (6 mg QD, 12 mg QD, and placebo) as a separate predictor (see FIG. 26, FIG. 27, and Table 32A for ACR20 results; see FIG. 29, FIG. 30 and Table 33A for PASI75 results) and also with the deucravacitinib treatment arms combined (see FIG. 28 and Table 32B for ACR20 results; see FIG. 31 and Table 33B for PASI75 results).

TABLE 32A
Logistic regression odds ratio results for correlating
ACR20 response status with baseline CRP level

| | | Odds Ratio or | | Confidence | |
|---|---|---|---|---|---|
| | | Interaction | | Interval | |
| Group | Comparison | Term | p-value | lower | upper |
| All | 6 mg QD vs | 2.456 | 0.012 | 1.214 | 4.96 |
| | placebo | | | | |
| All | 12 mg QD | 3.609 | <0.0001 | 1.759 | 7.41 |
| | vs placebo | | | | |
| CRP high | 6 mg QD vs | 2.167 | 0.126 | 0.805 | 5.83 |
| | placebo | | | | |
| CRP high | 12 mg QD | 5.140 | 0.003 | 1.776 | 14.88 |
| | vs placebo | | | | |
| CRP low | 6 mg QD vs | 2.756 | 0.047 | 1.012 | 7.51 |
| | placebo | | | | |
| CRP low | 12 mg QD | 2.676 | 0.054 | 0.984 | 7.28 |
| | vs placebo | | | | |
| Interaction | 6 mg QD vs | 0.786 | 0.738 | 0.192 | 3.22 |
| term: | placebo: | | | | |
| treatment | CRP high | | | | |
| arm* CRP | versus CRP | | | | |
| group | low | | | | |
| Interaction | 12 mg QD | 1.921 | 0.381 | 0.446 | 8.27 |
| term: | vs placebo: | | | | |
| treatment | CRP high | | | | |
| arm*CRP | versus CRP | | | | |
| group | low | | | | |

The results shown in FIG. 26, FIG. 27, and Table 32A indicate no differences in treatment benefit between CRP-high and CRP-low groups. CRP-high and CRP-low groups showed a treatment benefit (as indicated by a greater odds of achieving ACR20 with treatment, compared with placebo) at both deucravacitinib doses. The treatment benefit (as indicated by increased odds of responding to treatment compared to the odds of responding to placebo) did not reach statistical significance in the CRP-high group in the 6 mg treatment arm only (see third row) but was significant in the 12 mg treatment arm (see fourth row) and was significant in the CRP-low group at both doses (see fifth and sixth rows). The treatment benefit (odds ratio of responding to deucravacitinib versus placebo) did not differ significantly between CRP-high and CRP-low groups at either treatment dose (the interaction terms were not statistically significant; see last two rows of Table 32 A).

TABLE 32B
Logistic regression odds ratio results for correlating
ACR20 response status with baseline CRP level
(deucravacitinib treatment arms combined)

| | | Odds Ratio or | | Confidence | |
|---|---|---|---|---|---|
| | | Interaction | | Interval | |
| Group | Comparison | Term | p-value | lower | upper |
| All | Deucravacitinib | 2.961 | 0.0006 | 1.588 | 5.519 |
| | vs placebo | | | | |
| CRP high | Deucravacitinib | 3.215 | 0.0097 | 1.326 | 7.793 |
| | vs placebo | | | | |
| CRP low | Deucravacitinib | 2.715 | 0.0261 | 1.126 | 6.544 |
| | vs placebo | | | | |
| Interaction | Deucravacitinib | 1.184 | 0.7908 | 0.340 | 4.125 |
| term: | vs placebo: | | | | |
| treatment | CRP high | | | | |
| arm * CRP | versus CRP | | | | |
| group | low | | | | |

The ACR20 results presented in FIG. 28 and Table 32B indicate that the treatment benefit (odds ratio of responding to deucravacitinib versus placebo) was significant in CRP high and CRP low groups (see second and third rows of Table 32B), and the treatment benefit did not differ significantly between CRP high and CRP low groups (see last row of Table 32B).

TABLE 33A
Logistic regression odds ratio results for correlating
PASI75 response status with baseline CRP level

| | | Odds Ratio or | | Confidence | |
|---|---|---|---|---|---|
| | | Interaction | | Interval | |
| Group | Comparison | Term | p-value | lower | upper |
| All | 6 mg QD vs | 2.88 | 0.014 | 1.240 | 6.69 |
| | placebo | | | | |
| Al | 12 mg QD | 5.78 | <0.0001 | 2.436 | 13.72 |
| | vs placebo | | | | |
| CRP high | 6 mg QD vs | 3.54 | 0.040 | 1.060 | 11.85 |
| | placebo | | | | |
| CRP high | 12 mg QD | 9.78 | 0.001 | 2.616 | 36.59 |
| | vs placebo | | | | |
| CRP low | 6 mg QD vs | 2.12 | 0.221 | 0.636 | 7.11 |
| | placebo | | | | |
| CRP low | 12 mg QD | 3.72 | 0.028 | 1.153 | 11.99 |
| | vs placebo | | | | |
| Interaction | 6 mg QD vs | 1.67 | 0.557 | 0.302 | 9.19 |
| term: | placebo: CRP | | | | |
| treatment | high versus | | | | |
| arm*CRP | CRP low | | | | |
| group | | | | | |
| Interaction | 12 mg QD | 2.63 | 0.282 | 0.451 | 15.35 |
| term: | vs placebo: | | | | |
| treatment | CRP high | | | | |
| arm*CRP | versus CRP | | | | |
| group | low | | | | |

FIG. 29, FIG. 30, and Table 33A provide the results of an analogous analysis for PASI75 response. The results indicate that the CRP-high and CRP-low groups did not show consistent differences in responding to TYK2 inhibitor therapy at either deucravacitinib dose. Patients in the CRP-high group achieved a significant treatment response (as indicated by significantly greater odds of responding to deucravacitinib compared to the odds of responding to placebo) at both doses (see the third and fourth rows). Patients in the CRP-low group also showed increased odds of responding to deucravacitinib compared to the odds of responding to placebo at both doses (see the fifth and sixth rows), and this increased odds reached statistical significance in the 12 mg QD arm (see sixth row). The interaction terms were not statistically significant and indicate that at both treatment doses, the treatment benefit (as measured by odds ratio of responding to deucravacitinib versus placebo) did not differ significantly between CRP-high and CRP-low groups at either dose.

TABLE 33B
Logistic regression odds ratio results for correlating
PASI75 response status with baseline CRP level
(deucravacitinib treatment arms combined)

| | | Odds Ratio or | | Confidence | |
|---|---|---|---|---|---|
| | | Interaction | | Interval | |
| Group | Comparison | Term | p-value | lower | upper |
| All | Deucravacitinib | 3.998 | 0.0004 | 1.866 | 8.563 |
| | vs placebo | | | | |
| CRP high | Deucravacitinib | 5.405 | 0.0032 | 1.762 | 16.581 |
| | vs placebo | | | | |
| CRP low | Deucravacitinib | 2.840 | 0.0511 | 0.995 | 8.107 |
| | vs placebo | | | | |
| Interaction | Deucravacitinib | 1.903 | 0.4113 | 0.410 | 8.832 |
| term: | vs placebo: | | | | |
| treatment | CRP high | | | | |
| arm * CRP | versus CRP | | | | |
| group | low | | | | |

The PASI75 results presented in FIG. 31 and Table 33B indicate that there was a treatment benefit (greater odds of responding to deucravacitinib versus placebo) in both the CRP high and CRP low groups (see second and third rows of Table 33B). The treatment benefit (odds ratio of responding to deucravacitinib versus placebo) did not differ significantly between CRP high and CRP low groups (see last row of Table 33B).

The above logistic regression model did not adjust for baseline disease activity. To account for any potential confounding effects of baseline disease activity, a second model that adjusted for baseline disease activity was used. FIGS. 32-35 provide forest plots summarizing the results of this second model; Tables 34 and 35 provide the odds ratio results for CRP in this model.

TABLE 34
Logistic regression odds ratio results for correlating ACR20 response
status with baseline CRP level, adjusted for baseline DAS28

| | | Odds Ratio or | | Confidence | |
|---|---|---|---|---|---|
| | | Interaction | | Interval | |
| Group | Comparison | Term | p-value | lower | upper |
| All | 6 mg QD vs | 2.443 | 0.013 | 1.207 | 4.94 |
| | placebo | | | | |
| All | 12 mg QD | 3.618 | <0.0001 | 1.763 | 7.48 |
| | vs placebo | | | | |
| CRP-high | 6 mg QD vs | 2.168 | 0.126 | 0.805 | 5.84 |
| | placebo | | | | |
| CRP-high | 12 mg QD | 5.154 | 0.003 | 1.770 | 15.01 |
| | vs placebo | | | | |
| CRP-low | 6 mg QD vs | 2.752 | 0.048 | 1.008 | 7.51 |
| | placebo | | | | |
| CRP-low | 12 mg QD | 2.670 | 0.055 | 0.978 | 7.29 |
| | vs placebo | | | | |
| Interaction | 6 mg QD vs | 0.788 | 0.740 | 0.192 | 3.23 |
| term: | placebo: | | | | |
| treatment | CRP-high | | | | |
| arm*CRP | versus CRP- | | | | |
| group | low | | | | |
| Interaction | 12 mg QD | 1.930 | 0.382 | 0.442 | 8.43 |
| term: | vs placebo: | | | | |
| treatment | CRP-high | | | | |
| arm*CRP | versus CRP- | | | | |
| group | low | | | | |

Table 34 presents the results of the second model for ACR20 response status; the table provides the odds ratio results for ACR20 response status at week 16, adjusting for baseline DAS28. The first two rows in Table 34 provide the odds ratio and p-value for achieving ACR20 response, when adjusting for baseline DAS28, for all patients (CRP-high and CRP-low) using the following: ACR20 (Y/N)=treatment arm+TNF-inhibitor use+baseline weight (continuous)+baseline DAS28 (continuous). The odds ratios represent the odds ratios comparing 6 mg or 12 mg versus placebo. These results show that the odds of achieving an ACR20 response was significantly higher in the deucravacitinib treatment arms than in the placebo arm, even after adjusting for baseline DAS28.

The results provided in the third through sixth rows in Table 34 were obtained using the following: ACR20 (Y/N)=treatment arm*CRP group+TNF-inhibitor use+baseline weight (continuous)+baseline DAS28 (continuous). These results indicate that for CRP-high patients, after adjusting for baseline DAS28, the odds of achieving ACR20 response in each of the deucravacitinib treatment arms was greater than the odds of achieving ACR20 response in the placebo arm (see third and fourth rows), and the odds ratio reached statistical significance in the higher dose treatment arm (see fourth row). For CRP-low patients, after adjusting for baseline DAS28, the odds of achieving ACR20 response in each of the deucravacitinib treatment arms was significantly greater than the odds of achieving ACR20 response in the placebo arm (see fifth and sixth rows).

The results provided in the seventh and eighth rows in Table 34 were obtained using the same model that was used for the third through sixth rows. These results from the interaction terms were not statistically significant and indicate that in both treatment arms, the odds ratio of achieving ACR20 following TYK2 inhibitor treatment versus placebo in the CRP-high group was not statistically different from the odds ratio of achieving ACR20 following TYK2 inhibitor treatment versus placebo in the CRP-low group, even after adjusting for baseline DAS28.

TABLE 35
Logistic regression odds ratio results for correlating PASI75 response
status with baseline CRP level, adjusted for baseline PASI score

| | | Odds Ratio or | | Confidence | |
|---|---|---|---|---|---|
| | | Interaction | | Interval | |
| Group | Comparison | Term | p-value | lower | upper |
| All | 6 mg QD vs | 3.05 | 0.011 | 1.293 | 7.20 |
| | placebo | | | | |
| Al | 12 mg QD | 6.49 | <0.0001 | 2.665 | 15.82 |
| | vs placebo | | | | |
| CRP-high | 6 mg QD vs | 3.82 | 0.033 | 1.115 | 13.09 |
| | placebo | | | | |
| CRP-high | 12 mg QD | 12.05 | <0.0001 | 3.095 | 46.93 |
| | vs placebo | | | | |
| CRP-low | 6 mg QD vs | 2.29 | 0.183 | 0.676 | 7.74 |
| | placebo | | | | |
| CRP-low | 12 mg QD | 3.79 | 0.028 | 1.156 | 12.41 |
| | vs placebo | | | | |
| Interaction | 6 mg QD vs | 1.67 | 0.560 | 0.298 | 9.37 |
| term: | placebo: | | | | |
| treatment | CRP-high | | | | |
| arm*CRP | versus CRP- | | | | |
| group | low | | | | |
| Interaction | 12 mg QD | 3.18 | 0.206 | 0.528 | 19.16 |
| term: | vs placebo: | | | | |
| treatment | CRP-high | | | | |
| arm*CRP | versus CRP- | | | | |
| group | low | | | | |

Table 35 presents the results of the analogous model for PASI75 response status; the table provides the odds ratio results for PASI75 status at week 16, adjusting for baseline PASI score. The first two rows in Table 11 provide the odds ratio and p-value for achieving PASI75 response, when adjusting for baseline PASI score, for all patients (CRP-high and CRP-low) using the following: PASI75 (Y/N)=treatment arm+TNF-inhibitor use+baseline weight (continuous)+baseline PASI score (continuous). The odds ratios represent the odds ratios comparing 6 mg or 12 mg versus placebo. These results show that the odds of achieving a PASI75 response was significantly higher in the deucravacitinib treatment arms than in the placebo arm, even after adjusting for baseline PASI score.

The results provided in the third through sixth rows of Table 35 were obtained using the following: PASI75 (Y/N)=treatment arm*CRP group+TNF-inhibitor use+baseline weight (continuous)+baseline PASI score (continuous). The third and fourth rows provide odds ratio results for the CRP-high group. For CRP-high patients, after adjusting for baseline PASI score, the odds of achieving PASI75 response in each deucravacitinib treatment arm was significantly greater than the odds of achieving PASI75 in the placebo arm.

These results indicate that for the CRP-high patients, after adjusting for baseline PASI score, the odds of achieving PASI75 in each of the deucravacitinib treatment arms was significantly greater than the odds of achieving PASI75 in the placebo arm (see third and fourth rows). For CRP-low patients, after adjusting for baseline PASI score, the odds of achieving PASI response in each of the deucravacitinib treatment arms was greater than the odds of achieving PASI75 response in the placebo arm (see fifth and sixth rows), and the odds ratio reached statistical significance in the 12 mg QD arm (see sixth row).

The results provided in the seventh and eighth rows in Table 35 were obtained using the same model that was used for the third through sixth rows. The results from the interaction terms show that in both treatment arms, the odds ratio of achieving PASI75 following TYK2 inhibitor treatment versus placebo did not differ significantly between the CRP-low and CRP-high groups, even after adjusting for baseline PASI.

For PASI75, an additional logistic regression model was fit to assess the predictive value of baseline CRP level and baseline PASI score, each dichotomized as described above. This model generated the odds ratio of each of the dosed arms versus placebo, for each of the four baseline CRP-PASI groups (i.e., CRP-high-PASI-high; CRP-high-PASI-low; CRP-low-PASI-high; CRP-low-PASI-low; see FIG. 42). This model was: PASI75 (Y/N)=treatment arm+baseline CRP-PASI group+treatment arm*baseline CRP-PASI group+TNF-inhibitor use+baseline weight (continuous). Table 36 presents the results of this model.

TABLE 36
Logistic regression odds ratio results for correlating PASI75
response status with baseline CRP level and baseline PASI

| | | Odds | | Confidence Interval | |
|---|---|---|---|---|---|
| Group | Comparison | Ratio | p-value | lower | upper |
| All | 6 mg QD vs | 2.880 | 0.014 | 1.240 | 6.689 |
| | placebo | | | | |
| Al | 12 mg QD vs | 5.781 | <0.0001 | 2.436 | 13.720 |
| | placebo | | | | |
| CRP-high- | 6 mg QD vs | 18.381 | 0.002 | 2.789 | 121.146 |
| PASI-high | placebo | | | | |
| CRP-high- | 12 mg QD vs | 17.226 | 0.003 | 2.566 | 115.652 |
| PASI-high | placebo | | | | |
| CRP-high- | 6 mg QD vs | 0.710 | 0.701 | 0.123 | 4.087 |
| PASI-low | placebo | | | | |
| CRP-high- | 12 mg QD vs | 5.115 | 0.097 | 0.746 | 35.079 |
| PASI-low | placebo | | | | |
| CRP-low- | 6 mg QD vs | 2.470 | 0.307 | 0.436 | 13.989 |
| PASI-high | placebo | | | | |
| CRP-low- | 12 mg QD vs | 16.368 | 0.006 | 2.227 | 120.299 |
| PASI-high | placebo | | | | |
| CRP-low- | 6 mg QD vs | 1.866 | 0.473 | 0.339 | 10.278 |
| PASI-low | placebo | | | | |
| CRP-low- | 12 mg QD vs | 1.197 | 0.843 | 0.202 | 7.098 |
| PASI-low | placebo | | | | |

In Table 36, the first two rows provide the odds ratios of comparing 6 mg QD or 12 mg QD versus placebo, for all patients, using the following: PASI75 (Y/N)=treatment arm+TNF-inhibitor use+baseline weight (continuous). The odds ratios of the third through tenth rows provide results from the following: PASI75 (Y/N)=treatment arm+baseline CRP-PASI group+treatment arm*baseline CRP-PASI group+TNF-inhibitor use+baseline weight (continuous). For example, in the third row, 18.381 is the odds ratio comparing the odds of achieving a response in the 6 mg QD treatment arm to the odds of achieving a response in the placebo arm for patients with a CRP level above median and a baseline PASI score above median.

The results in Table 36 indicate that in both treatment arms, the CRP-high-PASI-high group showed significantly greater odds of responding (as indicated by PASI75) to deucravacitinib compared to placebo (see third and fourth rows), whereas the CRP-high-PASI-low group did not. For CRP-low patients, only the CRP-low-PASI-high group receiving the higher dose showed a significant treatment benefit (see eighth row). These results indicate that a high CRP baseline level alone was not consistently associated with responsiveness to treatment.

### CONCLUSIONS

The results presented herein indicate that baseline levels of each of BD2, IL-19 and IL-17A can be used to predict responsiveness to TYK2 inhibitor treatment and to select PsA patients for treatment. High baseline levels of these biomarkers were associated with clinical responsiveness. Patients with higher baseline expression of IL-23 pathway biomarkers BD2, IL-19 and IL-17A were more likely to benefit from deucravacitinib compared with placebo in skin and joint manifestations of psoriatic arthritis.

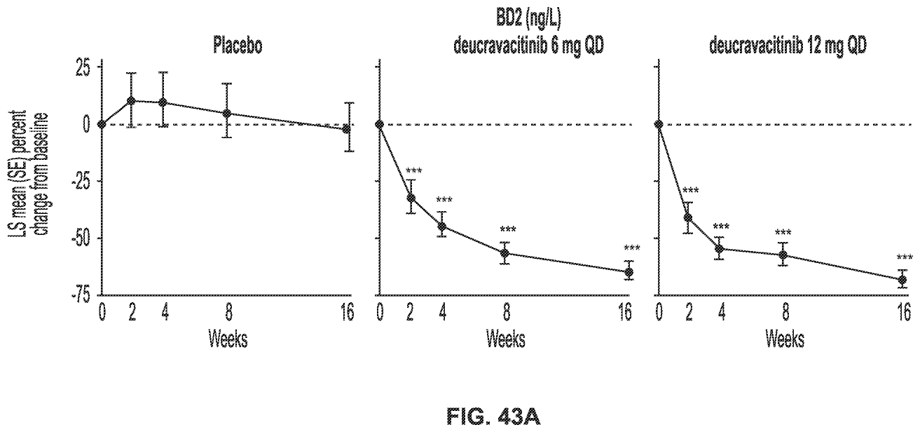

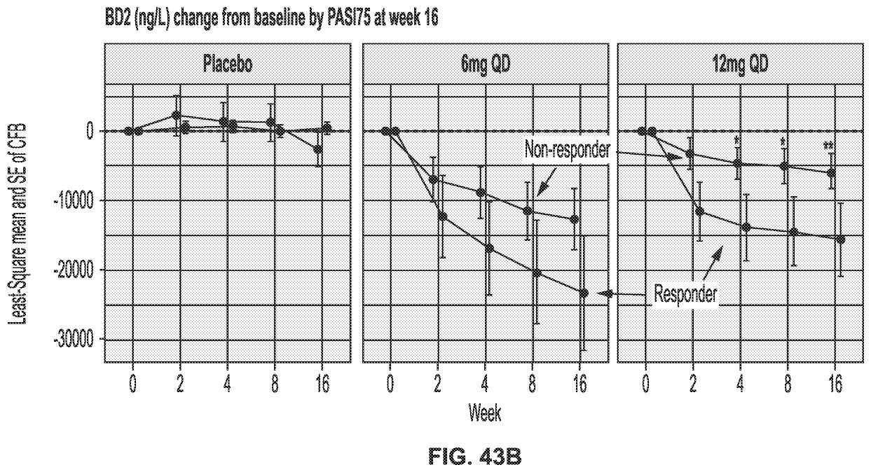

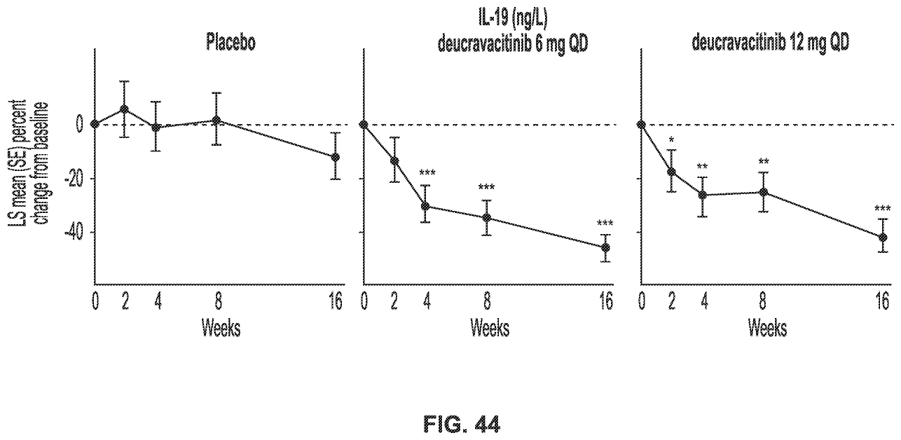

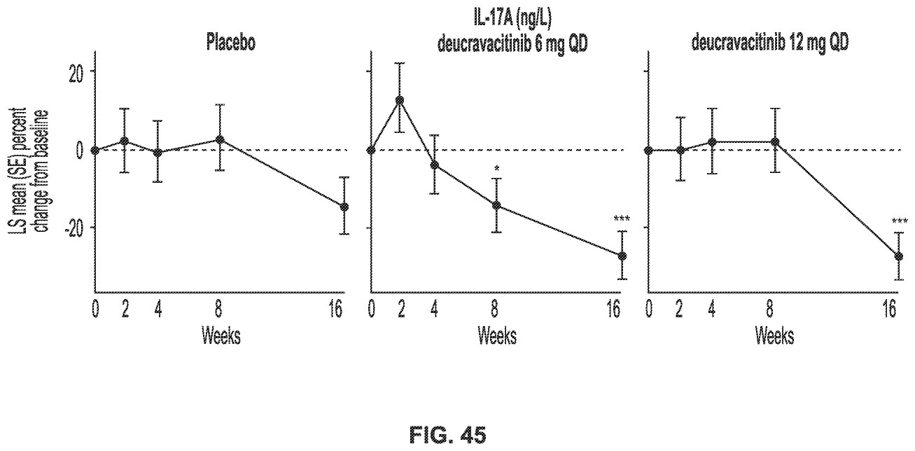

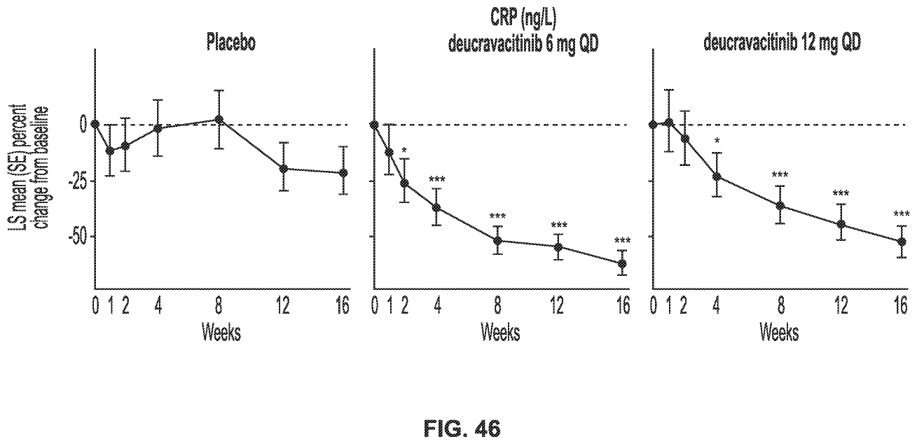

Furthermore, each of BD2, IL-19, and IL-17A decreased over time with TYK2 inhibitor treatment as shown in FIG. 43A and FIG. 43B, FIG. 44, and FIG. 45. Other proteins, such as CRP, also decreased over time with TYK2 inhibitor treatment (see FIG. 46); however, baseline levels of these proteins were not consistently associated with clinical responsiveness to TYK2 inhibitor treatment.

While this invention has been particularly shown and described with references to preferred embodiments thereof, it will be understood in light of the present disclosure by those skilled in the art that various changes in form and details may be made therein without departing from the scope of the invention encompassed by the appended claims.

What is claimed is:

1. A method for selecting a psoriatic arthritis patient for treatment with a TYK2 inhibitor, the method comprising:
(a) measuring a level of one or more proteins in a blood sample from a patient having psoriatic arthritis, wherein the one or more proteins are selected from β-defensin 2, IL-19, and IL-17A;
(b) for each level of protein measured in (a), comparing the level to a threshold level for the protein; and
(c) selecting the patient for treatment with a TYK2 inhibitor if the level of protein measured in (a) for at least one of the one or more proteins is greater than the threshold level for that protein.

2. The method according to claim 1, wherein the one or more proteins comprises β-defensin 2.

3. The method according to any one of claims 1-2, wherein the one or more proteins comprises IL-19.

4. The method according to any one of claims 1-3, wherein the one or more proteins comprises IL-17A.

5. The method according to any one of claims 1-4, wherein the blood sample is a serum sample or is a plasma sample.

6. The method according to any one of claims 1-5, further comprising (d) administering the TYK2 inhibitor to the patient if the patient is selected for treatment in (c).

7. The method according to any one of claims 1-6, wherein the TYK2 inhibitor is deucravacitinib.

8. A method for selecting a human subject having psoriatic arthritis for treatment with a TYK2 inhibitor, the method comprising:
(a) for each of two or more proteins selected from β-defensin 2, IL-19, and IL-17A, comparing the level of the protein in the human subject's blood to a threshold level for the protein; and
(b) if, for each of the two or more proteins, the level of protein in the human subject's blood is greater than the threshold level for the protein, selecting the human subject for treatment with the TYK2 inhibitor.

9. The method according to claim 8, wherein the two or more proteins comprise β-defensin 2 and IL-19.

10. The method according to claim 8, wherein the two or more proteins comprise β-defensin 2 and IL-17A.

11. The method according to claim 8, wherein the two or more proteins comprise IL-19 and IL-17A.

12. The method according to claim 8, wherein the two or more proteins comprise β-defensin 2, IL-19, and IL-17A.

13. The method according to any one of claims 8-12, wherein the TYK2 inhibitor is deucravacitinib.

14. The method according to any one of claims 8-13, further comprising administering the TYK2 inhibitor to the human subject if the human subject is selected for treatment with the TYK2 inhibitor.

15. A method of treating psoriatic arthritis in a human subject, the method comprising:
(a) measuring a level of one or more proteins in a blood sample from the human subject, wherein the one or more proteins are selected from β-defensin 2, IL-19, and IL-17A;
(b) for each level of protein measured in (a), comparing the level to a threshold level for the protein; and
(c) administering to the human subject a TYK2 inhibitor if the level of protein measured in (a) for at least one of the one or more proteins is greater than the threshold level for that protein.

16. The method according to claim 15, wherein the one or more proteins comprises β-defensin 2.

17. The method according to any one of claims 15-16, wherein the one or more proteins comprises IL-19.

18. The method according to any one of claims 15-17, wherein the one or more proteins comprises IL-17A.

19. The method according to any one of claims 15-18, wherein the blood sample is a serum sample or is a plasma sample.

20. The method according to any one of claims 15-19, wherein the TYK2 inhibitor is deucravacitinib.

21. The method according to any one of claims 15-19, wherein administering to the human subject a TYK2 inhibitor comprises administering deucravacitinib to the human subject at a dose of 6 mg or more per day.

22. The method according to any one of claims 15-19, wherein administering to the human subject a TYK2 inhibitor comprises administering deucravacitinib to the human subject at a dose of 12 mg or more per day.

23. A method of treating a human subject having psoriatic arthritis, the method comprising: identifying a human subject having psoriatic arthritis as having a level of one or more proteins in the blood that is above a threshold level, wherein the one or more proteins are selected from β-defensin 2, IL-19, and IL-17A, and wherein each of said proteins is associated with its own threshold level; and subsequently administering to the human subject a TYK2 inhibitor.

24. The method according to claim 23, wherein the TYK2 inhibitor is deucravacitinib.

25. A method comprising: administering a TYK2 inhibitor therapy to a patient having psoriatic arthritis, wherein prior to administering the TYK2 inhibitor therapy, the patient is identified as having one or more of the following:
a level of β-defensin 2 in the blood that is above a β-defensin 2 threshold level;
a level of IL-19 in the blood that is above an IL-19 threshold level;
a level of IL-17A in the blood that is above an IL-17A threshold level.

26. The method according to claim 25, wherein administering the TYK2 inhibitor therapy to the patient comprises administering deucravacitinib to the patient.

27. A TYK2 inhibitor therapy for treating psoriatic arthritis in a subject in need thereof, wherein the subject is identified as having: a level of β-defensin 2 in the blood that is above a β-defensin 2 threshold level, a level of IL-19 in the blood that is above an IL-19 threshold level, a level of IL-17A in the blood that is above an IL-17A threshold level, or any combination thereof.

28. A method for selecting a psoriatic arthritis patient for treatment with a TYK2 inhibitor, the method comprising:
(a) obtaining a level of one or more proteins in a blood sample from a patient having psoriatic arthritis, wherein the one or more proteins comprises one or more of β-defensin 2, IL-19, and IL-17A;
(b) selecting the patient for treatment with a TYK2 inhibitor if the level of protein obtained in (a) is greater than a threshold level for that protein.

29. The method according to claim 28, wherein the one or more proteins comprises β-defensin 2.

30. The method according to any one of claims 28-29, wherein the one or more proteins comprises IL-19.

31. The method according to any one of claims 28-30, wherein the one or more proteins comprises IL-17A.

32. A method for selecting a psoriatic arthritis patient for treatment with a TYK2 inhibitor, the method comprising:
(a) obtaining a level of β-defensin 2 in a blood sample from a patient having psoriatic arthritis; and
(b) selecting the patient for treatment with a TYK2 inhibitor if the level of β-defensin 2 obtained in (a) is greater than a β-defensin 2 threshold level.

33. A method for selecting a psoriatic arthritis patient for treatment with a TYK2 inhibitor, the method comprising:
(a) obtaining a level of IL-19 in a blood sample from a patient having psoriatic arthritis; and
(b) selecting the patient for treatment with a TYK2 inhibitor if the level of IL-19 obtained in (a) is greater than an IL-19 threshold level.

34. A method for selecting a psoriatic arthritis patient for treatment with a TYK2 inhibitor, the method comprising:
(a) obtaining a level of IL-17A in a blood sample from a patient having psoriatic arthritis; and
(b) selecting the patient for treatment with a TYK2 inhibitor if the level of IL-17A obtained in (a) is greater than an IL-17A threshold level.

35. The method according to any one of claims 28-34, further comprising administering the TYK2 inhibitor to the patient.

36. The method according to claim 35, wherein the TYK2 inhibitor is deucravacitinib.

37. The method according to claim 36, wherein the deucravacitinib is administered at a dose of 6 mg or more per day.

38. The method according to claim 36, wherein the deucravacitinib is administered at a dose of 12 mg or more per day.
